(19) United States
(12) Patent Application Publication
(10) Pub. No.: US 2017/0152263 A1
(43) Pub. Date: Jun. 1, 2017

(54) Substituted Indazole Compounds as IRAK4 Inhibitors

(71) Applicant: AURIGENE DISCOVERY TECHNOLOGIES LIMITED, Karnataka., Bangalore (IN)

(72) Inventors: Venkateshwar Rao Gummadi, Bangalore (IN); Susanta Samajdar, Bangalore (IN)

(21) Appl. No.: 15/320,111

(22) PCT Filed: Jun. 19, 2015
(86) PCT No.: PCT/IB2015/054620

§ 371 Date: Dec. 19, 2016

(30) Foreign Application Priority Data
Jun. 20, 2014 (IN) 3017/CHE/2014

(51) Int. Cl. C07D 487/04; C07D 401/14; C07D 471/04; C07D 413/14

(52) U.S. Cl. CPC C07D 487/04; C07D 413/14; C07D 401/14; C07D 471/04

(57) ABSTRACT

The present invention provides substituted indazole compound of formula (I) and pharmaceutically acceptable salts thereof, and their use to inhibit IRAK4 and/or for the treatment of diseases or disorders induced by IRAK4.

This application claims the benefit of Indian provisional application 3017/CHE/2014 filed on Jun. 20, 2014 which hereby incorporated by reference.

### FIELD OF THE INVENTION

This invention relates to compounds useful for treatment of cancer and inflammatory diseases associated with Interleukin-1 Receptor Associated Kinase (IRAK) and more particularly compounds that modulate the function of IRAK-4. The invention also provides pharmaceutically acceptable compositions comprising compounds of the present invention and methods of using said compositions in the treatment of diseases associated with IRAK-4.

### BACKGROUND OF THE INVENTION

Interleukin-1 (IL-1) Receptor-Associated Kinase-4 (IRAK-4) is a serine/threonine kinase enzyme that plays an essential role in signal transduction by Toll/IL-1 receptors (TIRs). Diverse IRAK enzymes are key components in the signal transduction pathways mediated by interleukin-1 receptor (IL-1R) and Toll-like receptors (TLRs) (Janssens, S, et al. Mol. Cell. 11(2), 2003, 293-302). There are four members in the mammalian IRAK family: IRAK-1, IRAK-2, IRAK-M and IRAK-4. These proteins are characterized by a typical N-terminal death domain that mediates interaction with MyD88-family adaptor proteins and a centrally located kinase domain. The IRAK proteins, as well as MyD88, have been shown to play a role in transducing signals other than those originating from IL-1R receptors, including signals triggered by activation of IL-18 receptors (Kanakaraj, et al. J. Exp. Med. 189(7), 1999, 1129-38) and LPS receptors (Yang, et al., J. Immunol. 163(2), 1999, 639-643). Out of four members in the mammalian IRAK family, IRAK-4 is considered to be the “master IRAK”. Under overexpression conditions, all IRAKs can mediate the activation of nuclear factor-KB (NF-KB) and stress-induced mitogen activated protein kinase (MAPK)-signaling cascades. However, only IRAK-1 and IRAK-4 have been shown to have active kinase activity. While IRAK-1 kinase activity could be dispensable for its function in IL-1-induced NF-KB activation (Kanakaraj et al, J. Exp. Med. 187(12), 1998, 2073-2079) and (Li, et al. Mol. Cell. Biol. 19(7), 1999, 4643-4652), IRAK-4 requires its kinase activity for signal transduction [(Li S, et al. Proc. Natl. Acad. Sci. USA 99(8), 2002, 5567-5572) and (Lye, E et al, J. Biol. Chem. 279(39); 2004, 40653-8)]. Given the central role of IRAK4 in Toll-like/IL-1R signalling and immunological protection, IRAK4 inhibitors have been implicated as valuable therapeutics in inflammatory diseases, sepsis and autoimmune disorders (Wietek C, et al, Mol. Interv. 2: 2002, 212-215).

Mice lacking IRAK-4 are viable and show complete abrogation of inflammatory cytokine production in response to IL-1, IL-18 or LPS (Suzuki et al. Nature, 416(6882), 2002, 750-756). Similarly, human patients lacking IRAK-4 are severely immunocompromised and are not responsive to these cytokines (Medvedev et al. J. Exp. Med., 198(4), 2003, 521-531 and Picard et al. Science 299(5615), 2003, 2076-2079). Knock-in mice containing inactive IRAK4 were completely resistant to lipopolysaccharide- and CpG-induced shock (Kim TW, et al. J. Exp. Med 204(5), 2007, 1025 -36) and (Kawagoe T, et al. J. Exp. Med. 204(5): 2007, 1013-1024) and illustrated that IRAK4 kinase activity is essential for cytokine production, activation of MAPKs and induction of NF-κB regulated genes in response to TLR ligands (Koziczak-Holbro M, et al. J. Biol. Chem. 282(18): 2007;13552-13560). Inactivation of IRAK4 kinase (IRAK4 KI) in mice leads to resistance to EAE due to reduction in infiltrating inflammatory cells into CNS and reduced antigen specific CD4+ T-cell mediated IL-17 production (Staschke et al. The Journal of Immunology, 183(1), 2009, 568-577).

The crystal structures revealed that IRAK-4 contains characteristic structural features of both serine/threonine and tyrosine kinases, as well as additional novel attributes, including the unique tyrosine gatekeeper residue. Structural analysis of IRAK-4 revealed the underlying similarity with kinase family; ATP-binding cleft sandwiched between a bilobal arrangements. The N-terminal lobe consists of mainly of a twisted five-stranded antiparallel beta-sheet and one alpha-helix, and the larger C-terminal lobe is predominantly alpha-helical. Yet, the structure reveals a few unique features for IRAK-4 kinase, including an additional alpha-helix from the N-terminal extension in the N-terminal lobe, a longer loop between helices alpha-D and alpha-E, and a significantly moved helix alpha G as well as its adjoining loops. The ATP-binding site in IRAK-4 has no deep pocket in the back but has a featured front pocket. This uniquely shaped binding pocket provides an excellent opportunity for designing IRAK-4 inhibitors.

The development of IRAK-4 kinase inhibitors has generated several novel classes of protein binders which includes thiazole and pyridine amides (George M Buckley, et al. Bioorg. Med. Chem. Lett., 18(11), 2008, 3211-3214), aminobenzimidazoles (Powers JP, et al. Bioorg. Med. Chem. Lett., 16(11), 2006, 2842-2845), Imidazo[1,2-a] pyridines (Buckley G M, et al. Bioorg. Med. Chem. Lett. 18(12), 2008, 3656-3660) and (Buckley G M, et al. Bioorg. Med. Chem. Lett. 18(11), 2008, 3291-3295), imidazo[1,2-b]pyridazines and benzimidazole-indazoles (WO2008030579; WO2008030584). Apparently, all of them are still in the early preclinical stage.

Despite various disclosures on different kinase inhibitors, however, with the rise in number of patients affected by kinase enzyme mediated diseases, there appears to be unmet need for newer drugs that can treat such diseases more effectively. There is still need for newer kinase inhibitors including multikinase inhibitors, which may be further useful in treatment of disorders owing to variations in various kinases activity and possessing broader role. They may also be useful as part of other therapeutic regimens for the treatment of disorders, alone or in combination with protein kinase compounds well known by the one skilled in the art.

### SUMMARY OF THE INVENTION

Provided herein is a compound of formula (I),

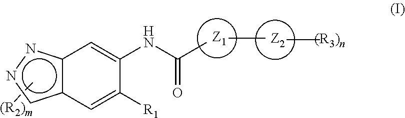

or a pharmaceutically acceptable salt or a stereoisomer thereof;

wherein,

Z₁ is optionally substituted cycloalkyl, optionally substituted aryl, optionally substituted heterocyclyl or is absent;

Z₂ is optionally substituted cycloalkyl, optionally substituted aryl or optionally substituted heterocyclyl;

R₁ is hydrogen, optionally substituted alkyl, amino, halogen, cyano, optionally substituted cycloalkyl, optionally substituted aryl, optionally substituted heterocyclyl, optionally substituted arylalkyl or optionally substituted heterocyclylalkyl;

R₂, at each occurrence, is hydrogen, halogen, amino, optionally substituted alkyl, optionally substituted cycloalkyl, optionally substituted aryl, optionally substituted heterocyclyl, optionally substituted arylalkyl or optionally substituted heterocyclylalkyl;

R₃, at each occurrence, is hydroxyl, halogen, optionally substituted alkyl, optionally substituted alkoxy, optionally substituted cycloalkyl or —NRₐR_{b};

Rₐ and R_{b}, independently, for each occurrence, are hydrogen, optionally substituted alkyl, optionally substituted acyl, optionally substituted cycloalkyl, optionally substituted aryl, optionally substituted heterocyclyl, optionally substituted arylalkyl or optionally substituted heterocyclylalkyl;

m, at each occurrence, is 0, 1 or 2; and

n, at each occurrence, is 0, 1, or 2.

In yet another aspect, the present invention provides a pharmaceutical composition comprising the compound of formula (I) or a pharmaceutically acceptable salt or a stereoisomer thereof, and at least one pharmaceutically acceptable excipient (such as a pharmaceutically acceptable carrier or diluent).

In yet further aspect, the present invention provides a use of a compound of formula (I) or a pharmaceutically acceptable salt or a stereoisomer thereof for the treatment and prevention of a disease or a disorder mediated by IRAK4 enzyme.

More particularly, the invention relates to the use of compound of formula (I) or a pharmaceutically acceptable salt or a stereoisomer thereof including mixtures thereof in all ratios as a medicament, by inhibiting IRAK or IRAK4 other related kinases.

The compound of formula (I) of the present invention possess the therapeutic role of inhibiting IRAK-1 or IRAK4-related kinases, which are useful in the treatment of diseases and/or disorders including, but not limited to, cancers, allergic diseases and/or disorders, autoimmune diseases and/or disorders, inflammatory diseases and/or disorder and/or conditions associated with inflammation and pain, proliferative diseases, hematopoietic disorders, hematological malignancies, bone disorders, fibrosis diseases and/or disorders, metabolic disorders and/or diseases, muscle diseases and/or disorders respiratory diseases and/or disorders, pulmonary disorders, genetic developmental diseases and/or disorders, neurological and neurodegenerative diseases and/or disorders, chronic inflammatory demyelinating neuropathies, cardiovascular, vascular or heart diseases and/or disorders, ophthalmic/ocular diseases and/or disorders, wound repair, infection and viral diseases. Therefore, inhibition of one or more of kinases would have multiple therapeutic indications.

### DETAILED DESCRIPTION OF THE INVENTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the term “optionally substituted” refers to the replacement of one to six hydrogen radicals on the same carbon or on different carbons in a given structure with the radical of a specified substituent including, but not limited to: hydroxyl, hydroxyalkyl, alkoxy, alkoxyalkyl, halogen, alkyl, aryl, aryloxy, aralkyl, heteroaryl, heteroaryloxy, heteroaralkyl, cycloalkyl, cycloalkoxy, (cycloalkyl)alkyl, heterocyclyl, (heterocyclyl)alkyl, amino, aminoalkyl, alkylamino, dialkylamino, acyl, —C(O)2H, —O(acyl), —NH(acyl), —N(alkyl)(acyl), cyano, phosphinate, phosphate, phosphonate, sulfonate, sulonamido, sulfate, haloalkyl or haloalkoxy. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C4 straight-chain alkyl groups or C₁-C4 branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” refers to a group R—CO— wherein R is an optionally substituted alkyl group defined above. Examples of ‘acyl’ groups are, but not limited to, CH₃CO—, CH₃CH₂CO—, CH₃CH₂CH₂CO— or (CH₃)₂CHCO—.

As used herein, the term “alkoxy” refers to a straight or branched, saturated aliphatic C₁-C₁₀ hydrocarbon radical bonded to an oxygen atom that is attached to a core structure. Preferably, alkoxy groups have one to six carbon atoms. Examples of alkoxy groups include but are not limited to methoxy, ethoxy, propoxy, isopropoxy, butoxy, isobutoxy, tert-butoxy, pentoxy, 3-methyl butoxy and the like.

As used herein, the term “haloalkyl” refers to alkyl group (as defined above) is substituted with one or more halogens. A monohaloalkyl radical, for example, may have a chlorine, bromine, iodine or fluorine atom. Dihalo and polyhaloalkyl radicals may have two and more of the same or different halogen atoms respectively. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃) or trifluoroethoxy (—OCH₂CF₃).

As used herein, the term “aryl” alone or in combination with other term(s) means a 6- to 10-membered carbocyclic aromatic system containing one or two rings wherein such rings may be fused. The term “fused” means that the second ring is attached or formed by having two adjacent atoms in common with the first ring. The term “fused” is equivalent to the term “condensed”. Examples of aryl groups include but are not limited to phenyl, naphthyl or indanyl. Unless otherwise specified, all aryl groups described herein may be optionally substituted.

The terms “amine” and “amino” are art-recognized and refer to both unsubstituted and substituted amines and salts thereof, e.g., a moiety that can be represented by

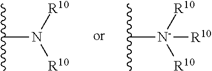

wherein each R¹⁰ independently represents a hydrogen or a hydrocarbyl group, or two R¹⁰ are taken together with the N atom to which they are attached complete a heterocycle having from 4 to 8 atoms in the ring structure.

As used herein, “aminoalkyl” refers to an amino group, as defined above, in which one or two hydrogen atoms are substituted with alkyl group.

As used herein, “nitro” refers to an —NO₂ group.

As used herein, “alkylamino” and “cycloalkylamino”, refer to an —N-group, wherein nitrogen atom of said group being attached to alkyl or cycloalkyl respectively. Representative examples of an “Alkylamino” and “Cycloalkylamino” groups include, but are not limited to —NHCH₃ and —NH-cyclopropyl. An amino group can be optionally substituted with one or more of the suitable groups.

As used herein the term “cycloalkyl” alone or in combination with other term(s) means C₃-C₁₀ saturated cyclic hydrocarbon ring. A cycloalkyl may be a single ring, which typically contains from 3 to 7 carbon ring atoms. Examples of single-ring cycloalkyls include cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused, and spirocyclic carbocyclyls.

As used herein, the term “cyano” refers to —CN group.

As used herein, the term “hydroxy” or “hydroxyl” refers to —OH group.

As used herein the term “hydroxyalkyl” or “hydroxylalkyl” means alkyl substituted with one or more hydroxyl groups, wherein the alkyl groups are as defined above. Examples of “hydroxyalkyl” include but are not limited to hydroxymethyl, hydroxyethyl, hydroxypropyl, propan-2-ol and the like.

As used herein, the term “halo” or “halogen” alone or in combination with other term(s) means fluorine, chlorine, bromine or iodine.

As used herein, the term “heterocycloalkyl” refers to a non-aromatic, saturated or partially saturated, monocyclic or polycyclic ring system of 3 to 15 member having at least one heteroatom or heterogroup selected from O, N, S, S(O), S(O)₂, NH or C(O) with the remaining ring atoms being independently selected from the group consisting of carbon, oxygen, nitrogen, and sulfur. The term “heterocycloalkyl” also refers to the bridged bicyclic ring system having at least one heteroatom or heterogroup selected from O, N, S, S(O), S(O)₂, NH or C(O). Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, aza-bicyclooctanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocycloalkyl” includes azetidinyl, pyrrolidinyl, morpholinyl and piperidinyl. All heterocycloalkyl are optionally substituted by one or more aforesaid groups.

As used herein, the term “heteroaryl” refers to an aromatic heterocyclic ring system containing 5 to 20 ring atoms, suitably 5 to 10 ring atoms, which may be a single ring (monocyclic) or multiple rings (bicyclic, tricyclic or polycyclic) fused together or linked covalently. Preferably, “heteroaryl” is a 5- to 6-membered ring. The rings may contain from 1 to 4 heteroatoms selected from N, O and S, wherein the N or S atom is optionally oxidized or the N atom is optionally quarternized. Any suitable ring position of the heteroaryl moiety may be covalently linked to the defined chemical structure.

Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, pyrazolopyrimidyl, furopyridinyl, purinyl, benzothiadiazolyl, benzooxadiazolyl, benzotriazolyl, benzotriadiazolyl, carbazolyl, dibenzothienyl, acridinyl and the like. Preferably “heteroaryl” refers to 5- to 6-membered ring selected from the group consisting of furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl and pyridazinyl. More preferably, pyrazolyl, pyridyl, oxazolyl and furanyl. All heteroaryls are optionally substituted by one or more aforesaid groups.

As used herein, the term “heterocyclyl” includes definitions of “heterocycloalkyl” and “heteroaryl”.

As used herein, the term ‘arylalkyl’ or ‘heterocyclylalkyl’ refers to an alkyl group which is further substituted by aryl or heterocyclyl respectively, wherein aryl, heterocyclyl and alkyl are as above defined.

As used herein, the term ‘compound(s)’ comprises the compounds disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

The phrase “pharmaceutically acceptable” refers to compounds or compositions that are physiologically tolerable and do not typically produce allergic or similar untoward reaction, including but not limited to gastric upset or dizziness when administered to mammal

The term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I)) can form pharmaceutically acceptable salts with various organic bases such as lysine, arginine, guanidine, diethanolamine or metformin. Suitable base salts include, but are not limited to, aluminum, calcium, lithium, magnesium, potassium, sodium, or zinc, salts.

As used herein, the term “stereoisomer” is a term used for all isomers of individual compounds of compound of formula (I) that differ only in the orientation of their atoms in space. The term stereoisomer includes minor image isomers (enantiomers) of compound of formula (I), mixtures of minor image isomers (racemates, racemic mixtures) of compound of formula (I), geometric (cis/trans or E/Z, R/S) isomers of compound of formula (I) and isomers of compound of formula (I) with more than one chiral center that are not minor images of one another (diastereoisomers).

The term “treatment”/“treating” means any treatment of a disease in a mammal, including: (a) Inhibiting the disease, i.e., slowing or arresting the development of clinical symptoms; and/or (b) Relieving the disease, i.e., causing the regression of clinical symptoms and/or (c) alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal, and most preferably a human.

As used herein, the term, “therapeutically effective amount” refers to an amount of a compound of formula (I) or a pharmaceutically acceptable salt or a stereoisomer thereof; or a composition comprising the compound of formula (I) or a pharmaceutically acceptable salt or a stereoisomer thereof, effective in producing the desired therapeutic response in a particular patient suffering from a disease or disorder mediated by kinase enzymes, particularly IRAK or IRAK4 enzyme. Particularly, the term “therapeutically effective amount” includes the amount of the compound of formula (I) or a pharmaceutically acceptable salt or a stereoisomer thereof, when administered, that induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

In certain embodiments, the present invention provides the compound of formula (I)

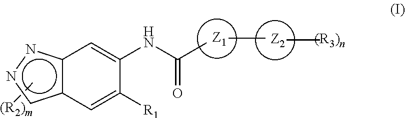

or a pharmaceutically acceptable salt or a stereoisomer thereof;

wherein,

Z₁ represents optionally substituted cycloalkyl, optionally substituted aryl, optionally substituted heterocyclyl or is absent;

Z₂ represents optionally substituted cycloalkyl, optionally substituted aryl or optionally substituted heterocyclyl;

R₁ is hydrogen, optionally substituted alkyl, amino, halogen, cyano, optionally substituted cycloalkyl, optionally substituted aryl, optionally substituted heterocyclyl, optionally substituted arylalkyl or optionally substituted heterocyclylalkyl;

R₂ at each occurrence is amino, optionally substituted alkyl, optionally substituted cycloalkyl, optionally substituted aryl, optionally substituted heterocyclyl, optionally substituted arylalkyl or optionally substituted heterocyclylalkyl;

R3 at each occurrence is hydroxyl, halogen, optionally substituted alkyl, optionally substituted alkoxy, optionally substituted cycloalkyl or —NRₐR_{b};

Rₐ and R_{b}, independently for each occurrence, are hydrogen, optionally substituted alkyl, optionally substituted acyl, optionally substituted cycloalkyl, optionally substituted aryl, optionally substituted heterocyclyl, optionally substituted arylalkyl or optionally substituted heterocyclylalkyl;

m, at each occurrence, is 0, 1 or 2; and

n, at each occurrence, is 0, 1, or 2.

In accordance with the foregoing embodiment, Z₁ is an optionally substituted heterocyclyl.

In certain embodiments, Z₁ represents cycloalkyl, aryl, or heterocyclyl, optionally substituted by one or more substituents selected, independently for each occurrence, from hydroxyl, halogen, alkyl, cycloalkyl, or NRaRb.

In accordance with any of the foregoing embodiments, in certain embodiments of formula (I), Z₁ is an optionally substituted heteroaryl; wherein the optional substituent is alkyl or cycloalkyl.

In accordance with any of the foregoing embodiments, in certain embodiments of formula (I), Z₁ is tetrazolyl, thienyl, triazolyl, pyrrolyl, pyridyl, pyranyl, pyrazinyl, pyridazinyl, pyrimidyl, imidazolyl, oxadiazolyl, thiadiazolyl, thiazolyl, isothiazolyl, oxazolyl, furanyl, pyrazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and pyrazolopyrimidyl; each of which is optionally substituted.

In accordance with any of the foregoing embodiments, in certain embodiments of formula (I), Z₁ is tetrazolyl, thienyl, triazolyl, pyrrolyl, pyridyl, pyranyl, pyrazinyl, pyridazinyl, pyrimidyl, imidazolyl, oxadiazolyl, thiadiazolyl, thiazolyl, isothiazolyl, oxazolyl, furanyl or pyrazolyl.

In accordance with any of the foregoing embodiments, in certain embodiments of formula (I), Z₁ is pyridyl or oxazolyl; wherein the oxazolyl group is optionally substituted with alkyl; in particular alkyl is methyl.

In accordance with any of the foregoing embodiments, in certain embodiments of formula (I), Z₁ is absent.

In certain embodiments, the present invention provides the compound of formula (I) or a pharmaceutically acceptable salt or a stereoisomer thereof; wherein, Z₂ is cycloalkyl, aryl or heterocyclyl.

In certain embodiments, Z₂ represents cycloalkyl, aryl, or heterocyclyl, optionally substituted by one or more substituents selected from hydroxyl, halogen, alkyl, alkoxyl, cycloalkyl, —NRₐR_{b}, or cycloalkoxy.

In accordance with any of the foregoing embodiments, in certain embodiments of formula (I), Z₂ is heterocyclyl.

In accordance with any of the foregoing embodiments, in certain embodiments of formula (I), Z₂ is azetidinyl, oxetanyl, furanyl, piperidinyl, morpholinyl, piperazinyl, thiomorpholinyl, 1,4-dioxanyl, tetrahydropyranyl, tetrahydrofuranyl, tetrahydropyridyl, tetrazolyl, thienyl, triazolyl, pyrrolyl, pyridyl, pyranyl, pyrazinyl, pyridazinyl, pyrimidyl, imidazolidinyl, imidazolyl, thiadiazolyl, thiazolyl, thiazolidinyl, isothiazolyl, oxadiazolyl, oxazolyl, pyrazolyl, pyrrolidinyl, oxazolidinyl, pyrazolidinyl, benzisoxazolyl, benzothiazolyl, benzofuranyl, benzothienyl, benzotriazinyl, indolyl, isoindolyl, indazolyl, quinolinyl, isoquinolinyl pyrrolopyridyl or pyrazolopyrimidyl.

In accordance with any of the foregoing embodiments, in certain embodiments of formula (I), Z₂ is pyridyl, piperazinyl, pyrimidyl, pyrrolidinyl, 1,2,3,4-tetrahydropyridyl, piperidinyl, pyrazolopyrimidyl or pyrrolopyridyl.

In certain embodiments, the compound of formula (I) is compound of formula (IA)

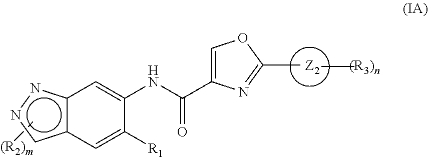

or a pharmaceutically acceptable salt thereof;
wherein, Z₂, R₁, R₂, R₃, ‘m’, and ‘n’ are as defined in compound of formula (I).

In certain embodiments, the compound of formula (I) is compound of formula (IB)

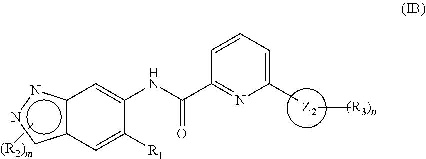

or a pharmaceutically acceptable salt thereof;
wherein, Z₂, R₁, R₂, R₃, ‘m’, and ‘n’ are as defined in compound of formula (I).

The embodiments below are illustrative of the present invention and are not intended to limit the claims to the specific embodiments exemplified.

In accordance with any of the foregoing embodiments, in certain embodiments of formula (I), the group

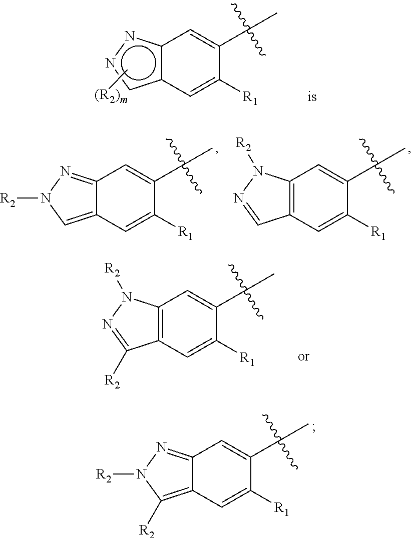

wherein R₁, R₂ and ‘m’ are as defined in compound of formula (I).

In accordance with any of the foregoing embodiments, in certain embodiments of formula (I), Z₂ is pyridyl.

In accordance with any of the foregoing embodiments, in certain embodiments of formula (I), Z₂ is pyrrolidinyl.

In accordance with any of the foregoing embodiments, in certain embodiments of formula (I), Z₂ is piperidinyl, piperazinyl, tetrahydropyridyl, pyrimidyl or pyrazolopyridyl.

In accordance with any of the foregoing embodiments, in certain embodiments of formula (I), R₁ is hydrogen, optionally substituted alkyl, amino, halogen, cyano, optionally substituted cycloalkyl, optionally substituted aryl, optionally substituted heterocyclyl, optionally substituted arylalkyl or optionally substituted heterocyclylalkyl.

In certain embodiments, R₁ is alkyl, cycloalkyl, aryl, heterocyclyl, arylalkyl, optionally substituted with one or more substituents selected, independently for each occurrence, from hydroxyl, halogen, alkyl, or hydroxyalkyl.

In accordance with any of the foregoing embodiments, in certain embodiments of formula (I), R₁ is heterocyclyl; optionally substituted with halogen, hydroxyl or hydroxyalkyl.

In accordance with any of the foregoing embodiments, in certain embodiments of formula (I), R₁ is optionally substituted azetidinyl, piperidinyl, morpholinyl, pyrrolidinyl or azabicyclooctanyl.

In accordance with any of the foregoing embodiments, in certain embodiments of formula (I), R₁ is piperidinyl, optionally substituted with hydroxyl.

In accordance with any of the foregoing embodiments, in certain embodiments of formula (I), R₁ is pyrrolidinyl, optionally substituted with hydroxyl.

In accordance with any of the foregoing embodiments, in certain embodiments of formula (I), R₂, at each occurrence, is amino, optionally substituted alkyl, optionally substituted cycloalkyl, optionally substituted aryl, optionally substituted heterocyclyl, optionally substituted arylalkyl or optionally substituted heterocyclylalkyl.

In certain embodiments, R₂ is alkyl, cycloalkyl, aryl, heterocyclyl, arylalkyl, or heterocyclylalkyl, optionally substituted with one or more substituents selected, independently for each occurrence, from alkyl, cycloalkyl, or heterocyclyl.

In accordance with any of the foregoing embodiments, in certain embodiments of formula (I), R₂ is optionally substituted alkyl, preferably, methyl.

In accordance with any of the foregoing embodiments, in certain embodiments of formula (I), R₂ is optionally substituted cycloalkyl, preferably, cyclopropyl.

In accordance with any of the foregoing embodiments, in certain embodiments of formula (I), R₂ is hydrogen.

In accordance with any of the foregoing embodiments, in certain embodiments of formula (I), R₃, at each occurrence, is hydroxyl, halogen, optionally substituted alkyl, optionally substituted alkoxy, optionally substituted cycloalkyl or —NRₐR_{b}; wherein Rₐ is hydrogen or optionally substituted alkyl; and R_{b} is hydrogen, optionally substituted alkyl, optionally substituted acyl, hydroxyalkyl or —SO₂-alkyl.

In accordance with any of the foregoing embodiments, in certain embodiments of formula (I), R₃, is —NRₐR_{b}; wherein Rₐ is hydrogen; and R_{b} is hydrogen or optionally substituted acyl.

In accordance with any of the foregoing embodiments, in certain embodiments of formula (I), Z₁ is optionally substituted pyridyl; Z₂ is pyrrolidinyl; R₁ is an optionally substituted groups selected from piperidinyl or pyrrolidinyl; R₂ is optionally substituted alkyl; R3 is halogen, alkyl, —NRₐR_{b}, hydroxyl or hydroxyalkyl; Rₐ is hydrogen or alkyl; and R_{b} is hydrogen or hydroxyalkyl.

In accordance with any of the foregoing embodiments, in certain embodiments of formula (I), Z₁ is oxazolyl; Z₂ is pyridyl, pyrimidyl or pyrrolidinyl, piperidinyl, tetrahydropyridyl, piperazinyl, pyrrolopyridyl; R₁ is an optionally substituted group selected from piperidinyl or pyrrolidinyl; R₂ is optionally substituted alkyl or cyclopropyl; R3 is halogen, alkyl, alkoxy, —NRₐR_{b}, hydroxyl, hydroxyalkyl optionally substituted cyclopropyl; Rₐ is hydrogen or alkyl; and R_{b} is hydrogen, alkyl, acyl, hydroxyalkyl, —SO₂-alkyl or optionally substituted cycloalkyl.

In accordance with any of the foregoing embodiments, in certain embodiments of formula (I), ‘m’ is 0.

In accordance with any of the foregoing embodiments, in certain embodiments of formula (I), ‘m’ is 1.

In accordance with any of the foregoing embodiments, in certain embodiments of formula (I), ‘m’ is 2.

In accordance with any of the foregoing embodiments, in certain embodiments of formula (I), ‘n’ is 0.

In accordance with any of the foregoing embodiments, in certain embodiments of formula (I), ‘n’ is 1.

In accordance with any of the foregoing embodiments, in certain embodiments of formula (I), ‘n’ is 2.

In certain embodiments, the present invention relates to a process for preparing indazole compound of formula (I).

### Pharmaceutical Compositions

In one certain embodiment, the present invention provided provides a pharmaceutical composition comprising the compound as disclosed herein, optionally admixed with and a pharmaceutically acceptable carrier or diluent.

As used herein, the term “composition” is intended to encompass a product comprising the specified ingredients in the specified amounts, as well as any product which results, directly or indirectly, from combination of the specified ingredients in the specified amounts.

As used herein, the term “pharmaceutical composition” refers to a composition(s) containing a therapeutically effective amount of at least one compound of formula (I) or its pharmaceutically acceptable salt; and a conventional pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

The present invention also provides methods for formulating the disclosed compounds as for pharmaceutical administration.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of formula (I) and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. The examples of carriers, stabilizers and adjuvants can be found in literature, Osol, A. and J.E. Hoover, et al.(eds.), Remington's Pharmaceutical Sciences, 15^{th} Ed., Easton, Mack Publ. Co., PA [1975].

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as the compounds of the present invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein refers to a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious or hazardous to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein mean the modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13^{th} ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The compounds of the present invention may be administered in combination with one or more other drugs (1) to complement and/or enhance prevention and/or therapeutic efficacy of the preventive and/or therapeutic drug effect of the compound of the present invention, (2) to modulate pharmacodynamics, improve absorption improvement, or reduce dosage reduction of the preventive and/or therapeutic compound of the present invention, and/or (3) to reduce or ameliorate the side effects of the preventive and/or therapeutic compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The respective compounds may be administered by the same or different route and the same or different method.

A concomitant medicine comprising the compounds of the present invention and other drug may be administered as a combination preparation in which both components are contained in a single formulation, or administered as separate formulations. The administration by separate formulations includes simultaneous administration and or administration of the formulations separated by some time intervals. In the case of the administration with some time intervals, the compound of the present invention can be administered first, followed by another drug or another drug can be administered first, followed by the compound of the present invention, so long as the two compounds are simultaneously active in the patient at least some of the time during the conjoint therapy. The administration method of the respective drugs may be administered by the same or different route and the same or different method.

The dosage of the other drug can be properly selected, based on a dosage that has been clinically used, or may be a reduced dosage that is effective when administered in combination with a compound of the present invention. The compounding ratio of the compound of the present invention and the other drug can be properly selected according to age and weight of a subject to be administered, administration method, administration time, disorder to be treated, symptom and combination thereof. For example, the other drug may be used in an amount of 0.01 to 100 parts by mass, based on 1 part by mass of the compound of the present invention. The other drug may be a combination of two or more kind of arbitrary drugs in a proper proportion. The other drug that complements and/or enhances the preventive and/or therapeutic efficacy of the compound of the present invention includes not only those that have already been discovered, but those that will be discovered in future, based on the above mechanism.

Diseases on which this concomitant use exerts a preventive and/or therapeutic effect are not particularly limited. The concomitant medicine can be used to treat any diseases discussed herein, as long as it complements and/or enhances the preventive and/or therapeutic efficacy of the compound of the present invention.

For example, in the methods of the invention directed to the treatment of cancer, the compound of the present invention can be used with an existing chemotherapeutic conjointly using a single pharmaceutical composition or a combination of different pharmaceutical compositions concomitantly or in a mixture form. Examples of the chemotherapeutic include an alkylation agent, nitrosourea agent, antimetabolite, anticancer antibiotics, vegetable-origin alkaloid, topoisomerase inhibitor, hormone drug, hormone antagonist, aromatase inhibitor, P-glycoprotein inhibitor, platinum complex derivative, other immunotherapeutic drugs and other anticancer drugs. Further, it a compound of the invention can be used administered conjointly with a cancer treatment adjunct, such as a leucopenia (neutropenia) treatment drug, thrombocytopenia treatment drug, antiemetic and cancer pain intervention drug, concomitantly or in a mixture form. Chemotherapeutic agents that may be conjointly administered with compounds of the invention include: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, and vinorelbine.

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, with cryotherapy, or with any combination of these.

In certain embodiments, different compounds of the invention may be conjointly administered with one or more other compounds of the invention. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound of the invention provides a synergistic effect. In certain embodiments, conjointly administering one or more additional chemotherapeutics agents provides an additive effect.

In certain embodiments, the compounds of the present invention can be used as IRAK-4 inhibitor, in combination with the following drugs.
(1) disease-modifying anti-rheumatic drugs (DMARDs)

(a) penicillamine such as D-penicillamine and the like.

(b) aminosalicylic acid preparation such as sulfasalazine, mesalazine, olsalazine, balsalazide and the like.

(c) antimalarial drug such as chloroquine and the like.

(d) pyrimidine synthesis inhibitor such as leflunomide and the like.
(2) Non-steroidal anti-inflammatory drug (NSAIDs)

(a) classical NSAIDs such as tolmetin, levorphanol, etodolac, fenoprofen, meloxicam, ethenzamide, tenoxicam, phenacetin, meclofenamic acid, salicylic acid, oxaprozin, thiaprofenic acid, lornoxicam, nabumeton, acetaminophen, alcofenac, ulinastatin, sulpyrine, antipyrine, sodium salicylate, migrenin, aspirin, mefenamic acid, flufenamic acid, diclofenac sodium, hyaluronate sodium, loxoprofen sodium, phenylbutazone, indomethacin, camostat mesylate, ibuprofen, naproxen, flurbiprofen, fenbufen, pranoprofen, floctafenine, ketoprofen, piroxicam, epirizole, tiaramide hydrochloride, zaltoprofen, gabexate mesylate, aceclofenac, sulindac, colchicine, probenecid, sulfinpyrazone, benzbromarone, allopurinol, sodium aurothiomalate, morphine hydrochloride, atropine, scopolamine, morphine, pethidine, oxymorphone or a salt thereof and the like.

(b) cyclooxygenase inhibitor (COX-1 selective inhibitor, COX-2 selective inhibitor etc.)

such as salicylic acid derivatives (e.g., celecoxib, aspirin), etoricoxib, valdecoxib, diclofenac, indomethacin, loxoprofen and the like.

(c) nitric oxide-releasing NSAIDs

(d) JAK inhibitor such as ruxolitinib, tofacitinib and the like.
(3) Integrin inhibitors such as natalizumab, vedolizumab, AJT 300, TRK-170, E-6007 and the like.
(4) anti-cytokine drugs

(a) TNF inhibitor such as infliximab, adalimumab, etanercept, certolizumab pegol, golimumab, soluble TNF-a receptor, TNF-binding protein, anti-TNF-antibody and the like.

b) Interleukin-1 inhibitors such as anakinra (IL-1 RA), soluble interleukin-1 receptor and the like.

(c) interleukin-6 inhibitor such as tocilizumab (IL-6R), anti-interleukin-6 antibody and the like.

(d) interleukin-10 modulators

(e) interleukin-12/23 inhibitor such as ustekinumab, briakinumab (anti-interleukin-12/23 antibody) and the like.

(f) MAPK inhibitor such as BMS-582949 and the like.

(g) cytokine production inhibitors such as iguratimod, tetomilast and the like.

(h) TNF-converting enzyme inhibitors

(i) interleukin-β converting enzyme inhibitors such as VX-765.

(j) interleukin-6 antagonists such as HMPL-004.

(k) interleukin-8 inhibitors such as IL-8 antagonist, CXCR1 & CXCR2 antagonist, reparixin and the like.

(1) chemokine antagonists such as CCR9 antagonist (CCX-282, CCX-025) , MCP-1 antagonist and the like.

(m) interleukin-2 receptor antagonist such as denileukin, diftitox and the like.

(n) therapeutic vaccines such as TNF-a vaccine.

(o) antisense compound such as ISIS 104838.
(5) angiotensin converting enzyme inhibitors such as enalapril, captopril, ramipril, lisinopril, cilazapril, perindopril and the like.
(6) angiotensin II receptor antagonists such as candesartan, candesartan cilexetil, azilsartan, azilsartan medoxomil, valsartan, irbesartan, olmesartan, eprosartan and the like.
(7) Steroids such as dexamethasone, hexestrol, methimazole, betamethasone, triamcinolone, triamcinolone acetonide, fluocinonide, fluocinolone acetonide, predonisolone, methylpredonisolone, cortisone acetate, hydrocortisone, fluorometholone, beclomethasone dipropionate, estriol and the like.
(8) immunomodulators (immunosuppressant) such as methotrexate, cyclophosphamide, MX-68, atiprimod dihydrochloride, BMS-188667, CKD-461, rimexolone, cyclosporine, tacrolimus, gusperimus, azathiopurine, antilymphocyte serum, freeze-dried sulfonated normal immunoglobulin, erythropoietin, colony stimulating factor, interleukin, interferon and the like.
(9) Diuretic drugs such as hydrochlorothiazide, spironolactone, furosemide, indapamide, bendrofluazide, cyclopenthiazide and the like.
(10) Dihydroorotate dehydrogenase (DHODH) inhibitors
(11) H G-CoA reductase inhibitors atorvastatin, simvastatin and the like.
(12) β receptor antagonists such as carvedilol, metoprolol, atenolol and the like.
(13) Anti-platelet drug, anticoagulator such as heparin, aspirin, warfarin and the like.
(14) cardiotonic drugs such as digoxin, dobutamine and the like.
(15) phosphodiesterase IV(PDE IV) inhibitors such as roflumilast, CG-1.088 and the like.
(16) iNOS inhibitor such as VAS-203 and the like.
(17) kinase inhibitors such as those that target EGFR, VEGF, Bcr-Abl, BTK, PI3K, Syk and the like.

Other concomitant drugs besides the above-mentioned include, for example, antibacterial agent, antifungal agent, antibiotic, sedative, anesthetic, antidepressant, antiulcer drug, antiarrhythmic agent, antiprotozoal agent, hypotensive diuretic drug, anticoagulant, tranquilizer, antipsychotic, antitumor drug, hypolipidemic drug, muscle relaxant, antiepileptic drug, antitussive and expectorant drug, antiallergic drug, cardiac stimulants, hypotensive diuretic, therapeutic drug for arrhythmia, vasodilator, vasoconstrictor, therapeutic drug for diabetes, antinarcotic, vitamin, vitamin derivative, antiasthmatic, therapeutic agent for atopic dermatitis, therapeutic agent for pollakisuria/anischuria, antipruritic drug, therapeutic agent for allergic rhinitis, hypertensor, endotoxin-antagonist or -antibody, signal transduction inhibitor, inhibitor of anti-inflammatory mediator activity, inhibitor of inflammatory mediator activity, antibody to inhibit inflammatory mediator activity, antibody to inhibit anti-inflammatory mediator activity and the like.

### Method of Treatment

In certain embodiments, the present invention relates to a compound or a pharmaceutically acceptable salt or a stereoisomer thereof, for use as a medicament.

In a further embodiment, the present invention relates to a method of treating IRAK4 mediated disorders or diseases or condition in a subject comprising administering a therapeutically effective amount of a compound of formula (I) or (IA) or (IB).

In certain embodiments, the present invention relates to a method of treating disorders or diseases or condition mediated by MyD88 in a subject comprising administering a therapeutically effective amount of a compound of formula (I) or (IA) or (IB).

In certain embodiments, the IRAK-mediated disorder or disease or condition is selected from the group consisting of a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder and a CNS disorder.

In certain embodiments, the IRAK-mediated disorder or disease or condition is selected from the group consisting of a cancer, an inflammatory disorder, an autoimmune disease, metabolic disorder, a hereditary disorder, a hormone-related disease, immunodeficiency disorders, a condition associated with cell death, a destructive bone disorder, thrombin-induced platelet aggregation, liver disease, pathologic immune conditions involving T cell activation and a cardiovascular disorder.

In any one of the foregoing embodiments, the cancer or proliferative disorder is selected the group consisting of a solid tumor, benign or malignant tumor, carcinoma of the brain, kidney, liver, stomach, vagina, ovaries, gastric tumors, breast, bladder colon, prostate, pancreas, lung, cervix, testis, skin, bone or thyroid; sarcoma, glioblastomas, neuroblastomas, multiple myeloma, gastrointestinal cancer, a tumor of the neck and head, an epidermal hyperproliferation, psoriasis, prostate hyperplasia, a neoplasia, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, papillary carcinoma, seminoma, melanoma; hematological malignancies selected from leukemia, diffuse large B-cell lymphoma (DLBCL), activated B-cell-like DLBCL, chronic lymphocytic leukemia (CLL), chronic lymphocytic lymphoma, primary effusion lymphoma, Burkitt lymphoma/leukemia, acute lymphocytic leukemia, B-cell pro lymphocytic leukemia, lymphoplasmacytic lymphoma, Waldenstrom's macroglobulnemia (WM), splenic marginal zone lymphoma, intravascular large B-cell lymphoma, plasmacytoma and multiple myeloma.

In any one of the forgoing embodiments, the neurodegenerative disease is selected from the group consisting of Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy and graft versus host disease.

In any one of the forgoing embodiments, the inflammatory disorder is selected from the group consisting of ocular allergy, conjunctivitis, keratoconjunctivitis sicca, vernal conjunctivitis, allergic rhinitis, autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, primary biliary cirrhosis, uveitis (anterior and posterior), Sjogren's syndrome, interstitial lung fibrosis, psoriatic arthritis, systemic juvenile idiopathic arthritis, nephritis, vasculitis, diverticulitis, interstitial cystitis, glomerulonephritis (e.g. including idiopathic nephrotic syndrome or minimal change nephropathy), chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS) and osteoarthritis.

In a preferred embodiment, the present invention relates to a method of treating disorders or diseases or condition mediated by L265P somatic mutation of MyD88 in a subject comprising administering a therapeutically effective amount of a compound of formula (I) or (IA) or (IB).

Such disorders, diseases, or conditions associated with an MYD88 mutation include cancers, inflammatory disorders such as ulcerative colitis, autoimmune diseases, metabolic disorders, hereditary disorders, hormone-related diseases, immunodeficiency disorders, conditions associated with cell death, destructive bone disorders, thrombin-induced platelet aggregation, liver disease and cardiovascular disorder.

In any one of the foregoing embodiments, the diseases mediated by L265P somatic mutation of MyD88 are hematological tumors such as lymphoma. In preferred embodiments, the diseases mediated by L265P somatic mutation of MyD88 are Waldenstrom's macroglobulnemia or diffuse large B-cell lymphoma.

In certain embodiments, present invention provides the compounds of formula (I) or (IA) or (IB) or a pharmaceutically acceptable salt or a stereoisomer thereof, for use for the treatment of a cancer, an inflammatory disorder, an autoimmune disease, metabolic disorder, a hereditary disorder, a hormone-related disease, immunodeficiency disorders, a condition associated with cell death, a destructive bone disorder, thrombin-induced platelet aggregation, liver disease, pathologic immune conditions involving T cell activation and a cardiovascular disorder.

In certain embodiments, present invention provides the compounds of formula (I) or (IA) or (IB) or a pharmaceutically acceptable salt or a stereoisomer thereof, in the manufacture of a medicament for the treatment of cancer, an inflammatory disorder, an autoimmune disease, metabolic disorder, a hereditary disorder, a hormone-related disease, immunodeficiency disorders, a condition associated with cell death, a destructive bone disorder, thrombin-induced platelet aggregation, liver disease and a cardiovascular disorder.

An embodiment of the present invention provides the IRAK4 inhibitor compounds according to of formula (I) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. Moreover, by utilizing the procedures described in detail, one of ordinary skill in the art can prepare additional compounds of the present invention claimed herein. All temperatures are in degrees Celsius (° C.) unless otherwise noted.

In certain embodiments, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), 3H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the Schemes and/or in the Examples herein below, by substituting an isotopically labeled reagent for a non-isotopically labeled reagent.

The MS (Mass Spectral) data provided in the examples were obtained using the following equipment:

API 2000 LC/MS/MS/Triplequad,

Agilent (1100) Technologies/LC/MS/DVL/Singlequad and

Shimadzu LCMS-2020/Singlequad.

The NMR data provided in the examples were obtained using the equipment-¹H-NMR: Varian-300,400 and 600 MHz.

The abbreviations used in the entire specification may be summarized herein below with their particular meaning.

° C. (degree Celsius); δ (delta); % (percentage); Ac₂O (Acetic anhydride); (BOC)₂O (Bocanhydride); bs (Broad singlet); CDCl₃ (Deuteriated chloroform); CH₂Cl_{2/}DCM (Dichloromethane); DAST (Diethylaminosulfur trifluoride); DMF (Dimethyl formamide); DMSO (Dimethyl sulphoxide); DIPEA/DIEA (N, N-Diisopropyl ethylamine); DMAP (Dimethyl amino pyridine); (DMSO-d6 (Deuteriated DMSO); d (Doublet); dd (Doublet of doublet); EDCI.HCl (1-(3-Dimethyl aminopropyl)-3-carbodiimide hydrochloride); EtOAc (Ethyl acetate); EtOH (Ethanol); Fe (Iron powder); g or gm (gram); HATU (1-[bis(dimethylamino)methylene]-1H-1,2,3-triazolo[4,5-b]pyridinium 3-oxid hexafluorophosphate); H or H₂ (Hydrogen); H₂O (Water); HOBt (1-Hydroxy benzotriazole); H₂SO₄ (Sulphuric acid); HCl (Hydrochloric acid); h or hr (Hours); Hz (Hertz); HPLC (High-performance liquid chromatography); J (Coupling constant); K₂CO₃ (Potassium carbonate); KOAc (Potassium Acetate); KNO₃ (Potassium nitrate); LiOH (Lithium hydroxide); NaHMDS (Sodiumbis(trimethylsilyl)amide); MeOH/CH₃OH (Methanol); mmol (Millimol); M (Molar); ml (Millilitre); mg (Milli gram); m (Multiplet); mm (Millimeter); MHz (Megahertz); MS (ES) (Mass spectroscopy-electro spray); min (Minutes); NaH (Sodium hydride); NaHCO₃ (Sodium bicarbonate); Na₂SO₄ (Sodium sulphate); N₂ (Nitrogen); NMR (Nuclear magnetic resonance spectroscopy); NMP (N-Methyl-2-pyrrolidone); Pd/C (palladium carbon); Pd(PPh3)2Cl₂ (Bis(triphenylphosphine)palladium(II)dichloride); Pd(OAc)₂(Palladium diacetate); Pd(dppf)Cl₂ (1,1′-Bis(diphenylphosphino)ferrocene) palladium(II)dichloride; Pd₂(dba)₃ (Tris(dibenzylideneacetone)dipalladium(0)); RT (Room Temperature); RM (Reaction mixture); S (Singlet); TBAF (Tetra-n-butylammonium fluoride); TBDMS (Tertiary butyl dimethyl silyl chloride); TEA (Triethyl amine); TFA (Trifluoroaceticacid); TLC (Thin Layer Chromatography); THF (Tetrahydrofuran); TFA (Trifluoro acetic acid); t (Triplet); Zn(CN)₂ (Zinc Cyanide).

### Intermediates

### Intermediate 1

### (S)-2-(3-((tert-butoxycarbonyl)amino)pyrrolidin-1-yl)oxazole-4-carboxylic acid

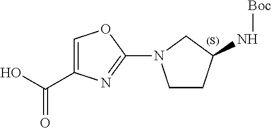

### Step 1: Preparation of ethyl (S)-2-(3-((tert-butoxycarbonyl)amino)pyrrolidin-1-yl)oxazole-4-carboxylate

The mixture of ethyl 2-chlorooxazole-4-carboxylate (100 mg, 0.5698 mmol), tert-butyl (S)-pyrrolidin-3-ylcarbamate (127 mg, 0.6837 mmol), DIPEA (0.284 mL, 1.4245 mmol) and DMF (5 mL) were heated at 120° C. for 2h. The reaction mass was quenched with ice water and extracted with DCM. The solvent was removed under reduced pressure to get the title compound (170 mg, 91.89%).

LCMS: %, m/z=270.1 (M-t-butyl+1).

### Step 2: Preparation of (S)-2-(3-((tert-butoxycarbonyl)amino)pyrrolidin-1-yl)oxazole-4-carboxylic acid

The solution of ethyl (S)-2-(3-((tert-butoxycarbonyl)amino)pyrrolidin-1-yl)oxazole-4-carboxylate (170 mg, 0.5224 mmol), lithium hydroxide (33 mg, 0.7837 mmol), in THF/methanol/water (10/1/2 mL), was stirred at RT for 12h. The reaction mixture was acidified with 2N HCl, the solvent was distilled and filtered the solid to get the title compound (150 mg, 96.77%).

LCMS: %, m/z=297.13.0 (M-t-butyl+1).

The following intermediates were prepared as per the procedure described in Intermediate 1 by using the same reaction conditions and appropriate reactants.

| Inter- | | |
|---|---|---|
| mediate | | |
| No. | Structure | Analytical Data |
| 7 | 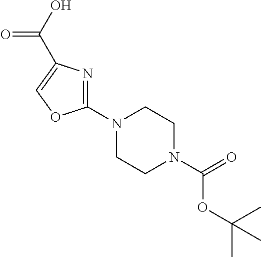 [SMILES: CC(C)(C)OC(=O)N1CCN(c2nc(C(=O)O)co2)CC1] | Yield: 20 mg (11%); LCMS: 98.04%, m/z = 298.3 (M + 1). |
| 8 | 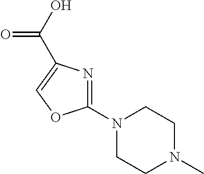 | Yield: (270 mg); LCMS: 99.6%, m/z = 212.0 (M + 1). |

### Intermediate 2

### (S)-2-(3-((tert-butyldimethylsilyl)oxy)pyrrolidin-1-yl)oxazole-4-carboxylic acid

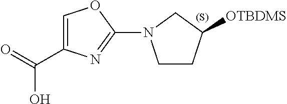

### Step 1: Preparation of ethyl (S)-2-(3-hydroxypyrrolidin-1-yl)oxazole-4-carboxylate

The title compound was prepared by reacting ethyl 2-chlorooxazole-4-carboxylate (500 mg, 2.8490 mmol) with (S)-pyrrolidin-3-ol (298 mg, 3.4188 mmol) according to the procedure described in Step-1 of intermediate 1. Yield: 535 mg (83.07%); LCMS: %, m/z=227.1 (M+1).

### Step 2: Preparation of ethyl (S)-2-(3-((tert-butyldimethylsilyl)oxy)pyrrolidin-1-yl)oxazole-4-carboxylate

To the solution of ethyl (S)-2-(3-hydroxypyrrolidin-1-yl)oxazole-4-carboxylate (535 mg, 2.3672 mmol) in DMF (10 mL), DMAP (29 mg, 0.2367 mmol), TBDMS chloride (429 mg, 2.8407 mmol) and imidazole (396 mg, 5.8072 mmol) were added and the reaction mixture was stirred at RT for 2h to get the crude compound which was purified by 60-120 silica gel column chromatography using 20% ethyl acetate in hexane as eluent to obtain the title compound (520 mg, 64.5%). LCMS: %, m/z=341.2 (M+1).

### Step 3: Preparation of (S)-2-(3-((tert-butyldimethylsilyl)oxy)pyrrolidin-1-yl)oxazole-4-carboxylic acid

The solution of ethyl (S)-2-(3-((tert-butyldimethylsilyl)oxy)pyrrolidin-1-yl)oxazole-4-carboxylate (520 mg, 1.5294 mmol) was hydrolyzed according to the procedure described in step 2 of Intermediate 1 to obtain the title compound (350 mg, 73.37%).

¹HNMR (CDCl₃, 400 MHz): δ 7.88 (s, 1H), 4.55-4.50(s, 1H), 3.75-3.60 (m, 3H), 3.5-3.4 (d, 1H), 2.05-1.90 (m, 2H), 0.9 (s, 9H). LCMS: %, m/z=313.1 (M+1).

### Intermediate 3

### 2-(2-aminopyridin-4-yl)oxazole-4-carboxylic acid

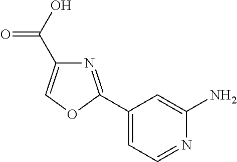 [SMILES: Nc1cc(-c2nc(C(=O)O)co2)ccn1]

### Step 1: Preparation of ethyl 2-(2-acetamidopyridin-4-yl)oxazole-4-carboxylate

To a solution of N-(5-(4,4,5,5 -tetramethyl-1,3 ,2-dioxaborolan-2-yl)pyridin-2-yl)acetamide (2.78 g, 10.04 mmol) in 1,2-dimethoxyethane (30 ml) under nitrogen was added ethyl 2-chlorooxazole-4-carboxylate (1 g, 7.09 mmol), sodium carbonate (106 mg, 21.2 mmol) in water (5 ml) and Pd(DPPF)Cl₂ (259 mg, 0.354 mmol) and heated to 90° C. for 4h to get the crude compound which was purifiedby 60-120 silica gel column chromatography using 50% ethyl acetate in hexane as eluent to obtain the title compound (680 mg, 36%). LCMS: 276.3 (M+1)⁺.

### Step-2: Preparation of 2-(2-aminopyridin-4-yl)oxazole-4-carboxylic acid

Ethyl 2-(2-acetamidopyridin-4-yl)oxazole-4-carboxylate (product of step 1 of intermediate 3) (900 mg, 3.27 mmol) was hydrolyzed using lithium hydroxide (329 mg, 7.85 mmol) in THF/methanol/water (30/1/5 mL) at RT for 4h to obtain the title compound (750 mg, 96%). ¹HNMR (DMSO-d₆), (300 MHz): δ 8.15(s,1H), 8.00(d,1H), 6.972-6.90(m,2H), 6.22(s,1H) LCMS: 97.8%, m/z=206.2 (M+1)

The following intermediates were prepared as per the procedure described in Intermediate 3 by using the same reaction conditions and appropriate reactants.

| Inter- | | |
|---|---|---|
| mediate | | |
| No. | Structure | Analytical Data |
| 4 | 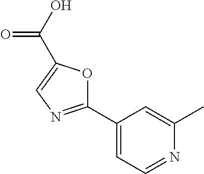 | Yield: 150 mg (85.5%); LCMS: 96.36%, m/z = 205.2 (M + 1) |
| 10 | 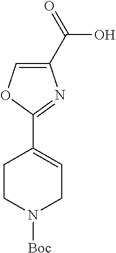 [SMILES: CC(C)(C)OC(=O)N1CC=C(c2nc(C(=O)O)co2)CC1] | Yield: 250 mg (92.9%); LCMS: 99.50%, m/z = 295.0 (M + 1) |
| 11 | 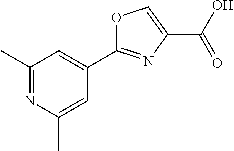 [SMILES: Cc1cc(-c2nc(C(=O)O)co2)cc(C)n1] | Yield: 400 mg (69.8%); m/z = 219.2 (M + 1)⁺. |
| 15 | 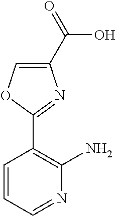 [SMILES: Nc1ncccc1-c1nc(C(=O)O)co1] | Yield: 120 mg (89.5%). LCMS: 96.6%; m/z = 206.0 (M + 1) |

### Intermediate 5

### 4-methyl-2-(2-methylpyridin-4-yl)oxazole-5-carboxylic acid

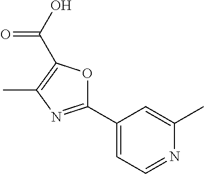

### Step 1: Preparation of ethyl 2-amino-4-methyloxazole-5-carboxylate

To a solution of ethyl 2-chloro-3-oxobutanoate (20 g, 12.1 mmol) and urea (24 g, 50.0 mmol) in methanol (120 ml) was heated to reflux for 36h. The solid obtained was filtered suspended in 2N sodium hydroxide and extracted with ethyl acetate. The organic layer was dried over Na₂SO₄ and concentrated under reduced pressure to obtain the title compound (1.8 g, 5%).

¹H NMR (DMSO-d₆), (300 MHz): δ 7.43(s, 2H), 4.18(q, 2H), 2.22(s, 3H), 1.24(t, 3H)

LCMS: 97.75%, m/z=171.2 (M+1)

### Step 2: Preparation of ethyl 2-chloro-4-methyloxazole-5-carboxylate

To a suspension of cupric chloride (822 mg, 0.611 mmol) and tert-butyl nitrite (578 mg 0.56 mmol) in acetonitrile (30 ml) was added ethyl 2-amino-4-methyloxazole-5-carboxylate (800 mg, 0.47 mmol) below 10° C. and stirred at RT for 2h. The reaction mixture was quenched with 2N HCl. The compound was extracted with diethyl ether and concentrated to get crude product, which was purified by column chromatography using 10% ethyl acetate in hexane to obtain the title compound (400 mg, 44.9%). LCMS: 94.66%, m/z=190.05 (M+1)

### Step 3: Preparation of 4-methyl-2-(2-methylpyridin-4-yl)oxazole-5-carboxylic acid

The title compound was prepared according to the procedure described in steps 1 and 2 of intermediate 3 by using the appropriate reactants and reaction conditions. Yield: 170 mg (98%).

¹HNMR (DMSO-d₆), (300 MHz): δ 8.65 (s, 1H), 7.80 (s, 1H), 7.71 (d, 1H), 3.95 (bs, 1H), 2.58 (s, 3H), 2.46 (s, 3H), LCMS: 97.8%, m/z=206.2 (M+1), HPLC: 98.4%.

### Intermediate 6

### (S)-6-(3-((tert-butyldimethylsilyl)oxy)pyrrolidin-1-yl)picolinic acid

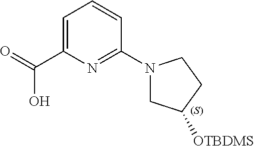

### Step 1: Preparation of methyl 6-bromopicolinate

To a solution of 6-bromopicolinic acid (5 g, 2.47 mmol) in methanol (35 ml), SOCl₂ (4.417 g, 3.7 mmol) was added at 0° C. and heated to reflux for 2h. The methanol was evaporated under reduced pressure and the compound was extracted with ethyl acetate, washed with NaHCO3 solution, dried over Na₂SO₄ and concentrated to obtain the title compound (5.2 g, 91%). ¹HNMR (DMSO-d₆), (300 MHz): δ8.11-8.05 (m, 1H) 7.99-7.91 (m, 1H) 7.82-7.79 (m, 1H) LCMS: 55.34%, m/z=218.1 (M+1).

### Step 2: Preparation of methyl (S)-6-(3-hydroxypyrrolidin-1-yl)picolinate

In a sealed tube, methyl 6-bromopicolinate (1 g, 0.462 mmol), (S)-pyrrolidin-3-ol (858 mg, 0.694 mmol), sodium carbonate (1.9 g, 1.85 mmol) and DMF (10 mL) were taken and heated at 140° C. for 4h to get the crude compound which was purified by 60-120 silica gel column chromatography using 1% methanol in DCM as eluent to obtain the title compound (500 mg, 49%). LCMS: 97.46%, m/z=223.2 (M+1)

### Step 3: Preparation of methyl (S)-6-(3-((tert-butyldimethylsilyl)oxy)pyrrolidin-1-yl)picolinate

The title compound was prepared according to the procedure described in step-2 of Intermediate 2 by reacting methyl (S)-6-(3-hydroxypyrrolidin-1-yl) picolinate (500 mg, 0.22 mmol) with TBDMS chloride (405 mg, 0.270 mmol). Yield: 400 mg (52.9%).

¹HNMR (DMSO-d₆), (300 MHz): δ7.52 (t, 1H), 7.40 (d, 1H), 6.12 (d, 1H), 4.54-4.52 (m, 1H), 3.93 (s, 3H), 3.70-3.57 (m, 3H), 3.40-3.35 (m, 2H) 2.09-1.96 (m, 2H) 1.46 (s, 3H), 0.90 (s, 9H), 0.02 (s, 6H)

### Step 4: Preparation of (S)-6-(3-((tert-butyldimethylsilyl) oxy) pyrrolidin-1-yl)picolinic acid

The title compound was prepared by hydrolyzing methyl (S)-6-(3-((tert-butyldimethylsilyl)oxy)pyrrolidin-1-yl)picolinate according to the procedure described in step-3 of Intermediate 2. Yield: 250 mg, (66%); LCMS: 95.41%, m/z=323.32 (M+1).

### Intermediate 7

### 2-(4-(tert-butoxycarbonyl)piperazin-1-yl)oxazole-4-carboxylic acid

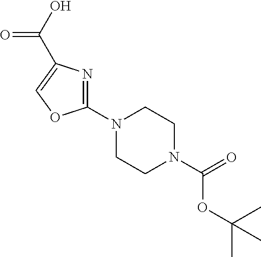 [SMILES: CC(C)(C)OC(=O)N1CCN(c2nc(C(=O)O)co2)CC1]

### Step 1: Preparation of ethyl 2-(4-(tert-butoxycarbonyl)piperazin-1-yl)oxazole-4-carboxylate

To a solution of tert-butyl piperazine-1-carboxylate (637 mg, 3.42 mmol) and ethyl 2-chlorooxazole-4-carboxylate (500 mg, 2.85 mmol) in DMF (10 ml), K₂CO₃ (771 mg 5.714 mmol) was added and stirred at RT for 5h. The reaction mixture was quenched by water, the compound was extracted with ethyl acetate and concentrated to obtain the title compound (380 mg, 41%). LCMS: 98.04%, m/z=277.2 (M-tert-butyl).

### Step 2: Preparation of 2-(4-(tert-butoxycarbonyl)piperazin-1-yl)oxazole-4-carboxylic acid

The solution of ethyl 2-(4-(tert-butoxycarbonyl)piperazin-1-yl)oxazole-4-carboxylate) (200 mg, 0.065 mmol), lithium hydroxide (100 mg, 0.24 mmol), THF/methanol/water (10/5/5 mL) were stirred at RT for 2h. The reaction mixture was acidified with 2N HCl, the solvent was distilled and filtered the solid to get the title compound (20 mg, 11%).

LCMS: 98.04%, m/z=298.3(M+1).

### Intermediate 8

### 2-(4-methylpiperazin-1-yl)oxazole-4-carboxylic acid

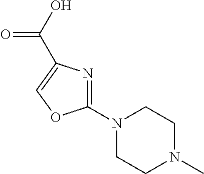

### Step 1: Preparation of ethyl 2-(4-methylpiperazin-1-yl)oxazole-4-carboxylate

To a solution of 1-methylpiperazine (1 g, 5.71 mmol) and ethyl 2-chlorooxazole-4-carboxylate (0.7 g, 6.85 mmol) in DMF (15 ml) K₂CO₃ (1.5 g, 11.42 mmol) was added and stirred at RT for 5h. The reaction mixture was quenched by water, the compound was extracted with ethyl acetate and concentrated to obtain the title compound (450 mg, 33%). LCMS: 93.9%, m/z=240.3(M+1)

### Step 2: Preparation of 2-(4-methylpiperazin-1-yl)oxazole-4-carboxylic acid

The solution of ethyl 2-(4-methylpiperazin-1-yl)oxazole-4-carboxylate (250 mg, 0.10 mmol), lithium hydroxide (100 mg, 0.24 mmol), THF/methanol/water (10/5/5 mL) was stirred at RT for 2h, acidified with 2N HCl, distilled the solvent and filtered the solid to obtain the title compound. Yield: 270 mg (crude). LCMS: 99.6%, m/z=212.0(M+1).

### Intermediate 9

### 2-(2-cyclopropylpyridin-4-yl) oxazole-4-carboxylic acid

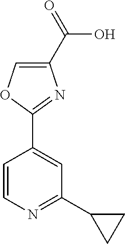 [SMILES: O=C(O)c1coc(-c2ccnc(C3CC3)c2)n1]

### Step 1: Preparation of methyl 2-cyclopropylisonicotinate

To a solution of methyl 2-chloroisonicotinate (2 g, 1.17 mmol) in 1,4-Dioxane (30 ml) under nitrogen cyclopropylboronic acid (1.5 g,1.7 mmol), potassium carbonate (2.4 g, 1.70 mmol) in water (5 ml) and Pd(PPh3)4 (0.675 g. 0.050 mmol) were added and heated to 90° C. for 4h to obtain the crude compound Which was purified by 60-120 silica gel column chromatography using 50% ethyl acetate in hexane as eluent to obtain the title compound (0.8 g, 39.02%). LCMS: 90.3%, m/z=178.0 (M+1)

### Step 2: Preparation of 2-cyclopropylisonicotinic acid

The solution of methyl 2-cyclopropylisonicotinate (product of step 1 of intermediate 9) (800 mg, 0.451 mmol), lithium hydroxide (284 mg, 0.677 mmol), THF/methanol/water (20/10/10 mL) was stirred at RT for 2h. the reaction mixture was acidified with 2N HCl, and the solvent was distilled and filtered the solid to obtain the title compound (700 mg, 95.89%). LCMS: 97.66%, m/z=164.3 (M+1)

### Step 3: Preparation of methyl (2-cyclopropylisonicotinoyl)serinate

To a solution of 2-cyclopropylisonicotinic acid (product of step 2 of intermediate 9) (700 mg, 0.42 mmol) in DMF (5 mL) L-serine methyl ester (799 mg, 0.51 mmol), EDCI (1.23 g, 0.640 mmol), HOBt (57.9 mg, 0.042 mmol) and DIPEA (1.66 g, 1.28 mmol) were added. The reaction mixture was stirred for 12 h at room temperature. The DMF was evaporated completely under reduced pressure and the compound was extracted with ethyl acetate, dried over Na₂SO₄ and concentrated. The crude compound was washed with diethyl ether to obtain the title compound (700 mg, 62%). LCMS: 100%, m/z=265.2 (M+1)

### Step 4: methyl 2-(2-cyclopropylpyridin-4-yl)-4,5-dihydrooxazole-4-carboxylate

To a solution of methyl (2-cyclopropylisonicotinoyl)serinate (product of step 3 of intermediate 9) (700 mg, 0.26 mmol) in DCM (35 mL) DAST (747 mg, 0.463 mmol) was added at −70° C. drop wise and stirred at −55° C. for 2h. Then K₂CO₃ (1.27 g, 0.921 mmol) was added and the reaction mixture was stiffed at room temperature for 2h. The excess K₂CO₃ was filtered and the filtrate was taken to next step without purification. LCMS: 92.56%, m/z=247.3 (M+1)

### Step 5: methyl 2-(2-cyclopropylpyridin-4-yl)oxazole-4-carboxylate

To a solution of methyl 2-(2-cyclopropylpyridin-4-yl)-4,5-dihydrooxazole-4-carboxylate (product of step 4 of intermediate 9) (640 mg, 0.260 mmol) in DCM (35 mL), DBU (1.19 g, 0.780 mmol) and BrCCl3 (1.55 g, 0.780) were added at 0° C. and stirred at room temperature for 2h. The reaction mass was washed with NaHCO3 solution and brine solution, dried over Na₂SO₄ and purified by column chromatography using 30% ethyl acetate in hexane to obtain the title compound (400 mg, 66%) LCMS: 96.89%, m/z=245.1 (M+1)

### Step 6: 2-(2-cyclopropylpyridin-4-yl)oxazole-4-carboxylic acid

The solution of methyl 2-(2-cyclopropylpyridin-4-yl)oxazole-4-carboxylate (product of step 5 of intermediate 9) (400 mg, 0.155 mmol), lithium hydroxide (75 mg, 0.311 mmol), THF/methanol/water (20/10/10 mL) was stirred at RT for 2h, and the reaction mixture was acidified with 2N HCl. The excess solvent was distilled and the solid was filtered to obtain the title compound (356 mg, 100%). LCMS: 100%, m/z=231.3 (M+1)

The following intermediates were prepared as per the procedure described in Intermediate 1 by using the same reaction conditions and appropriate reactants.

| Inter- | | |
|---|---|---|
| mediate | | |
| No. | Structure | Analytical Data |
| 12 | 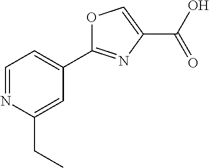 [SMILES: CCc1cc(-c2nc(C(=O)O)co2)ccn1] | Yield: 290 mg (98%). LCMS: 100%, m/z = 218.9 (M + 1) |
| 13 | 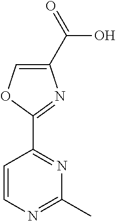 | Yield: 86 mg (97.7%). LCMS: 100%, m/z = 206.1 (M + 1) |

### Intermediate 14

### 2-(2-amino-3-fluoropyridin-4-yl)oxazole-4-carboxylic acid

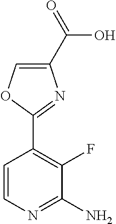 [SMILES: Nc1nccc(-c2nc(C(=O)O)co2)c1F]

### Step-1: Preparation of tert-butyl (4-chloro-3-fluoropyridin-2-yl)carbamate

To a solution of 2-bromo-4-chloro-3-fluoropyridine (825 mg, 3.92 mmol) in 1-4 Dioxane in a sealed tube (10 ml) tert-butyl carbamate (505 mg, 4.32 mmol) and caesium carbonate (2.30 g, 7.85 mmol) and Pd(dba) (3350 mg, 0.392 mmol) and xanthphos (230 mg, 0.392 mmol) were added under argon and the reaction mixture was stirred at 100° C. for 4h. The compound was extracted with ethyl acetate, dried over Na₂SO₄ and concentrated to get crude compound which was purified by column chromatography using 20% EtOAC in hexane to obtain the title compound (450 mg, 46.5%). LCMS: 63.4%, m/z=247.0 (M+1)

### Step-2: Preparation of tert-butyl (3-fluoro-4-(4,4,5,5-tetramethyl-1,3,2-dioxaborolan-2-yl)pyridin-2-yl)carbamate

To a solution of tert-butyl (4-chloro-3-fluoropyridin-2-yl)carbamate (product of step-1 of intermediate 14) (600 mg, 2.43 mmol) in 1,4-Dioxane (10 ml) under nitrogen, Bispinacalatodiboron (860 mg, 3.41 mmol), potassium acetate (470 mg, 4.87 mmol) and Pd(DDPF)Cl2 (170 mg, 170 mmol) were added and heated to 100° C. for 40 min. The reaction mixture was diluted with ethyl acetate, washed the EtOAc layer by water, dried over Na₂SO₄ and concentrated to get crude compound which was purified by combiflash chromatography using 3.5% methanol in chloroform to obtain the title compound (500 mg).

### Step-3: Preparation of 2-(2-amino-3-fluoropyridin-4-yl)oxazole-4-carboxylic acid

The title compound was prepared according to the procedure described in steps 1 and 2 of intermediate 3 by using the appropriate reactants and reaction conditions. Yield: 100 mg (92.5%) LCMS: 72.0%, m/z=224.6 (M+1).

### Intermediate 15

### 2-(2-aminopyridin-3-yl)oxazole-4-carboxylic acid

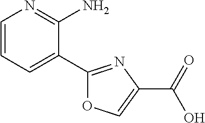 [SMILES: Nc1ncccc1-c1nc(C(=O)O)co1]

The title compound was prepared according to the procedure described in steps 1 and 2 of intermediate 3 by using the appropriate reactants and reaction conditions. Yield: 120 mg (89.5%). LCMS: 96.6%; m/z=206.0 (M+1).

### Intermediate 16

### 2-(2-acetamidopyridin-4-yl) oxazole-4-carboxylic acid

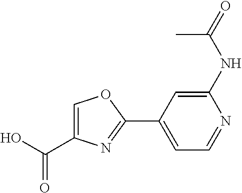 [SMILES: CC(=O)Nc1cc(-c2nc(C(=O)O)co2)ccn1]

Using the same reaction conditions as described in step 2 of intermediate 3, ethyl 2-(2-acetamidopyridin-4-yl)oxazole-4-carboxylate (product of step 1 of intermediate 3) (1 g, 0.363 mmol) was hydrolyzed using lithium hydroxide (152 mg, 0.363 mmol) in THF/methanol/water (20/5/5 mL) at RT for 30 min to obtain the title compound (780 mg, 87.6%) LCMS: 91.64%; m/z=248.01 (M+1).

### Intermediate 17

### 2-(1H-pyrrolo[2,3-b]pyridin-4-yl)oxazole-4-carboxylic acid

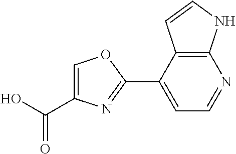 [SMILES: O=C(O)c1coc(-c2ccnc3[nH]ccc23)n1]

### Step-1: Preparation of 4-chloro-1-tosyl-1H-pyrrolo[2,3-b ]pyridine

To a solution of 4-chloro-1H-pyrrolo[2,3-b]pyridine (645 mg, 3.28 mmol) in toluene (10 ml), p-toluene sulfonyl chloride (689 mg, 3.61 mmol), tetrabutylammonium hydrogen sulphate (55 mg, 0.164) and NaOH (2 g, 52.63 mmol) solution in water, were added at 0° C. and stirred at room temperature for 12h. The reaction mass was diluted with ethyl acetate, separated the organic layer, dried over Na₂SO₄ and concentrated to obtain the crude compound which was purified by column chromatography using 10% EtOAc in hexane to get the title compound (852 mg, 74%). LCMS: 97.8%; m/z =307.1 (M+1).

### Step-2: Preparation of 4-(4,4,5,5-tetramethyl-1,3,2-dioxaborolan-2-yl)-1-tosyl-1H-pyrrolo[2,3-b]pyridine

Using the same reaction conditions as described in step-2 of Intermediate 14, 4-Chloro-1-tosyl-1H-pyrrolo[2,3-b]pyridine (850 mg, 2.43 mmol) was reacted with Pd(DDPF)Cl₂ (100 mg, 0.127 mmol) to obtain the title compound (753 mg, 78.2%); LCMS: 98.03%; m/z=399.2 (M+1).

### Step-3-: Preparation of 2-(1H-pyrrolo[2,3-b]pyridin-4-yl)oxazole-4-carboxylic acid

The title compound was prepared according to the procedure described in steps 1 and 2 of intermediate 3 by using the appropriate reactants and reaction conditions. Yield: 277 mg (91%); LCMS: 87.82%; m/z=230.2 (M−1).

### EXAMPLES

### Example 1

N-(1-methyl-5-(piperidin-1-yl)-1H-indazol-6-yl)-2-(2-methylpyridin-4-yl)oxazole-4-carboxamide hydrochloride

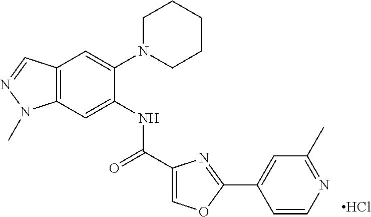 [SMILES: Cc1cc(-c2nc(C(=O)Nc3cc4c(cnn4C)cc3N3CCCCC3)co2)ccn1 ; Cl]

### Step-1: Preparation of 5-fluoro-6-nitro-1H-indazole

A mixture of 4-fluoro-2-methyl-5-nitroaniline (1.0 gm, 5.847 mmol), potassium acetate (690 mg, 7.0164 mmol) and acetic anhydride (1.8 gm, 17.543 mmol) in chloroform (30 mL) was heated at 40° C. for 0.5 h. At this temperature, isoamyl nitrite (1.37 gm, 11.694 mmol) was added and stirred at 80° C. for 12 h. After completion of reaction, solvent was removed under reduced pressure, the residue was basified with sodium carbonate solution and was extracted with ethyl acetate. The organic layer was washed with water followed by brine solution and concentrated under reduced pressure to obtain crude compound. The residue was purified by column chromatography over silica gel (30% EtOAc:Hexane) to give the pure compound which was stirred with methanolic HCl (60 mL) for 30 min. The reaction mixture was concentrated under reduced pressure, basified with aqueous sodium carbonate solution and extracted with ethyl acetate. The organic layer was washed with water, brine and was dried over anhydrous Na₂SO₄ and concentrated under reduced pressure to obtain crude compound (130 mg).

¹HNMR (DMSO-d₆, 300 MHz): δ 13.8 (s, 1H), 8.39-8.37 (d, 1H), 8.27 (s, 1H), 7.95-7.92 (d, 1H). LCMS: m/z=180.0 (M-1).

### Step-2: Preparation of 6-nitro-5-(piperidin-1-yl)-1H-indazole

A solution of 5-fluoro-6-nitro-1H-indazole (130 mg, 0.528 mmol) and piperidine (0.5 mL) in a sealed tube was stirred at 100° C. for 3h. After completion of reaction, reaction mixture was concentrated under reduced pressure to get the crude title product (70 mg).

LCMS: 90.32%, m/z=247.0 (M+1). Step-3: Preparation of 1-methyl-6-nitro-5-(piperidin-1-yl)-1H-indazole and 2-methyl-6-nitro-5-(piperidin-1-yl)-2H-indazole

To a solution of sodium hydride (390 mg, 8.13 mmol) in THF (10 mL) 6-nitro-5-(piperidin-1-yl)-1H-indazole (1 g, 4.065 mmol) was added at 0° C. After 15 min, at 0° C. methyl iodide (2.3 gm, 16.26 mmol) was added. The reaction mixture was allowed to room temperature for 2h. The reaction mixture was diluted with EtOAc, washed with brine and dried over anhydrous Na₂SO_{4.} This was purified by silica gel column chromatography and elution with 20% ethyl acetate in hexane gave isomer A; 1-methyl-6-nitro-5-(piperidin-1-yl)-1H-indazole (350 mg, 33.14%)

¹HNMR (CDCl₃, 400 MHz): δ 7.95 (s, 1H), 7.72 (s, 1H), 7.49 (s, 1H), 4.08 (s, 3H), 2.94-2.92 (t, 4H), 1.73-1.66 (m, 4H), 1.60-1.52 (m, 2H). LCMS: 99.15%, m/z=261.4 (M+1).

On further elution with 50% ethyl acetate in hexane gave isomer B; 2-methyl-6-nitro-5-(piperidin-1-yl)-2H-indazole (500 mg, 47.4%).

¹HNMR (CDCl₃, 400 MHz): δ 7.97 (s, 1H), 7.85 (s, 1H), 7.29 (s, 1H), 4.22 (s, 3H), 2.92-2.89 (t, 4H), 1.72-1.66 (m, 4H), 1.59-1.54 (m, 2H).LCMS: 97.53%, m/z=261.4 (M+1).

### Step-4: Preparation of 1-methyl-5-(piperidin-1-yl)-1H-indazol-6-amine

To a solution of 1-methyl-6-nitro-5-(piperidin-1-yl)-1H-indazole (350 mg, 1.346 mmol) in THF (20 mL) ammonium chloride (1.2 gm, 21.536 mmol) in water (5 mL) was added and zinc dust (700 mg, 10768 mmol) and stirred at RT for 30 min. The catalyst was filtered through Celite®, extracted with DCM (2*100 mL) and distilled out the solvent to obtain the crude compound (300 mg, 100%). LCMS: 99.49%, m/z=231.1 (M+1).

### Step-5: Preparation of N-(1-methyl-5-(piperidin-1-yl)-1H-indazol-6-yl)-2-(2-methylpyridin-4-yl)oxazole-4-carboxamide hydrochloride

To a solution of 1-methyl-5-(piperidin-1-yl)-1H-indazol-6-amine (100 mg, 0.434 mmol) in

DMF (5 mL) was added 2-(2-methylpyridin-4-yl)oxazole-4-carboxylic acid (89 mg, 0.434 mmol), EDCI (123 mg, 0.651 mmol), HOBt (88 mg, 0.651 mmol), DIPEA (168 mg, 1.302 mmol). The reaction mixture was stirred for 12 h at room temperature. After completion of reaction, reaction mixture was diluted with EtOAc, washed with brine and dried over anhydrous Na₂SO_{4.} This was then treated with methanolic HCl to obtain the title compound (75 mg, 38.5%).

¹HNMR (CD₃OD, 300 MHz): δ 9.05 (s, 1H), 8.95-8.92 (d, 1H), 8.61 (s, 1H), 8.54-8.52 (d, 1H), 8.20-8.10 (m, 3H), 4.11 (s, 3H), 3.80-3.40 (bs, 4H), 2.92 (s, 3H), 2.15-2.00 (m, 4H), 1.98-1.60 (bs, 2H). LCMS: 98.29%, m/z=417.2 (M+1). HPLC: 98.07%.

### Example 2

N—(2-methyl-5-(piperidin-1-yl)-2H-indazol-6-yl)-2-(2-methylpyridin-4-yl)oxazole-4-carboxamide hydrochloride

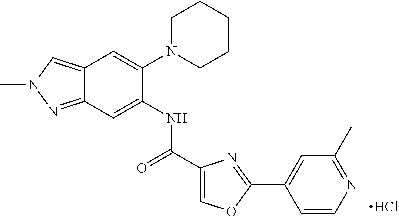

### Step-1: Preparation of 2-methyl-5-(piperidin-1-yl)-2H-indazol-6-amine

The title compound was prepared according to the procedure described in steps 4 of Example 1 by using the appropriate reactants and reaction conditions. Yield: 430 mg (97.7%). LCMS: 100%, m/z=231.2 (M+1).

Step-2: Preparation of N-(2-methyl-5-(piperidin-1-yl)-1H-indazol-6-yl)-2-(2-methylpyridin-4-yl)oxazole-4-carboxamide

The title compound was prepared according to the procedure described in steps 5 of Example 1 by using the appropriate reactants and reaction conditions. Yield: 100 mg (51.2%).

¹HNMR (CD₃OD, 300 MHz): δ 9.02 (s, 1H), 8.96-8.94 (d, 1H), 8.60 (s, 1H), 8.56 (s, 1H), 8.52-8.46 (d, 1H), 8.10-7.90 (bs, 1H), 4.32 (s, 3H), 3.80-3.40 (bs, 4H), 2.91 (s, 3H), 2.10-1.95 (m, 4H), 1.90-1.65 (bs, 2H). LCMS: 99.07%, m/z=417.2 (M+1). HPLC: 97.47%.

### Example 3

(S)-6-(3-hydroxypyrrolidin-1-yl)—N—(2-methyl-5-(piperidin-1-yl)-2H-indazol-6-yl)picolinamide

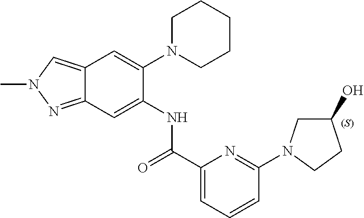

### Step-1: Preparation of 6-bromo-N-(2-methyl-5-(piperidin-1-yl)-2H-indazol-6-yl)picolinamide

To a solution of 2-methyl-5-(piperidin-1-yl)-2H-indazol-6-amine (250 mg, 1.08 mmol) in DMF (5 mL) was added 6-bromopicolinic acid (263 mg, 1.30 mmol), EDCI (311 mg, 1.63 mmol), HOBt (154 mg, 1.14 mmol) and DIPEA (420 mg, 3.26 mmol). The reaction mixture was stirred for 12 h at room temperature. After completion of reaction, reaction mixture was diluted with EtOAc, washed with brine and dried over anhydrous Na₂SO_{4.} After concentration under reduced pressure, the residue was purified by flash chromatography (CH2Cl₂: MeOH: 98.5:1.5) to obtain the title compound (300 mg, 66.6%). LCMS: 94.61%, m/z=414.1 (M+). HPLC: 92.21%.

### Step-2: Preparation of (S)-6-(3-hydroxypyrrolidin-1-yl)—N—(2-methyl-5-(piperidin-1-yl)-2H-indazol-6-yl)picolinamide

In a sealed tube, taken 6-bromo-N-(2-methyl-5-(piperidin-1-yl)-2H-indazol-6-yl)picolinamide (100 mg, 0.241 mmol), (S)-pyrrolidin-3-ol (32 mg, 0.362 mmol), sodium carbonate (102 mg, 0.966 mmol) and DMF (4 mL) and heated at 140° C. for 4h to get the crude product. Purification was done by 60-120 silica gel column chromatography using 1% methanol in DCM as eluent to obtain the title compound (60 mg, 60%).

¹HNMR (CDCl₃, 400 MHz): δ 10.94 (s, 1H), 8.90 (s, 1H), 7.74 (s, 1H), 7.64-7.62 (m, 2H), 7.29 (s, 1H), 6.56-6.54 (dd, 1H), 4.68 (s, 1H), 4.17 (s, 3H), 3.76-3.73 (m, 4H), 3.20-2.60 (bs, 4H), 2.24-2.15 (m, 2H), 1.90-1.75 (m, 6H). LCMS: 100%, m/z=421.4 (M+1). HPLC: 95.03%.

### Example 4

(S)-2-(3-aminopyrrolidin-1-yl)—N—(1-methyl-5-(piperidin-1-yl)-1H-indazol-6-yl)oxazole-4-carboxamide

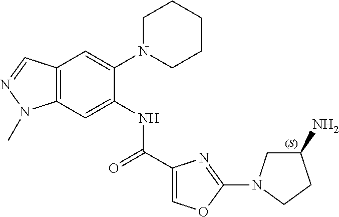

To a solution of 1-methyl-5-(piperidin-1-yl)-1H-indazol-6-amine (product of step 4 of example 1) in DMF (3 mL) (S)-2-(3-((tert-butoxycarbonyl)amino)pyrrolidin-1-yl)oxazole-4-carboxylic acid (intermediate 1) (154 mg, 0.521 mmol), EDCI (124 mg, 0.652 mmol), HOBt (88 mg, 0.652 mmol) and DIPEA (223 mg, 1.736 mmol) were added. The reaction mixture was stirred for 12 h at room temperature. After completion of reaction, reaction mixture was diluted with EtOAc, washed with brine and dried over anhydrous Na₂SO₄ and concentrated under reduced pressure to obtain the crude product. The crude product was dissolved in DCM (10 mL) and TFA/DCM (1/1 mL) was added and stirred at room temperature for 3 h. After completion of reaction, excess of solvent was removed under reduced pressure, basified with saturated sodium carbonate solution and diluted with ethyl acetate. The organic layer was washed with brine and dried over anhydrous Na₂SO₄. After concentration, the residue was purified by column chromatography (CH₂Cl₂: MeOH; 98:2) to obtain the title compound (90 mg, 56.2%).

¹HNMR (CDCl₃, 400 MHz): δ 10.5 (s, 1H), 8.64 (s, 1H), 7.84 (s, 2H), 7.43 (s, 1H), 4.04 (s, 3H), 3.76-3.74 (m, 3H), 3.70-3.60 (m, 1H), 3.31-3.29 (m, 1H), 3.10-3.00 (m, 2H), 2.80-2.65 (m, 2H), 2.30-2.20 (m, 1H), 2.10-1.70 (m, 6H). LCMS: 98.97%, m/z=410.2 (M+1). HPLC: 96.41%.

### Example 5

(S)-2-(3-aminopyrrolidin-1-yl)—N—(2-methyl-5-(piperidin-1-yl)-2H-indazol-6-yl)oxazole-4-carboxamide

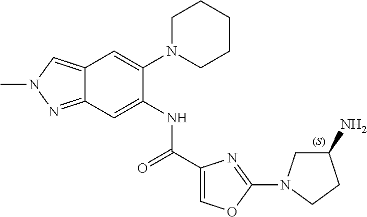

The title compound was prepared according to the procedure described in Example 4 by using the appropriate reactants and reaction conditions. Yield: 90 mg (56.2%).

¹HNMR (CDCl₃, 400 MHz): δ 10.40 (s, 1H), 8.82 (s, 1H), 7.85 (s, 1H), 7.73 (s, 1H), 7.29 (s, 1H), 4.16 (s, 3H), 3.80-3.70 (m, 3H), 3.65-3.58 (m, 1H), 3.29-3.27 (d, 1H), 3.20-3.00 (m, 2H), 2.80-2.60 (bs, 2H), 2.30-2.15 (m, 2H), 2.00-1.75 (m, 6H). LCMS: 99.64%, m/z=410.2 (M+1). HPLC: 96.59%.

### Example 6

(S)-2-(3-hydroxypyrrolidin-1-yl)—N—(2-methyl-5-(piperidin-1-yl)-2H-indazol-6-yl)oxazole-4-carboxamide

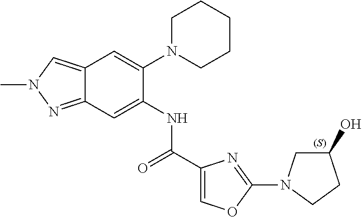

The title compound was prepared according to the procedure described in Example 4 by using the appropriate reactants and reaction conditions. Yield: 85 mg, (72.6%).

¹HNMR (CDCl₃, 300 MHz): δ 10.37 (s, 1H), 8.81 (s, 1H), 7.85 (s, 1H), 7.72 (s, 1H), 7.28 (s, 1H), 4.63 (s, 1H), 4.16 (s, 3H), 3.74-3.67 (m, 3H), 3.67-3.56 (m, 1H), 3.15-2.95 (bs, 2H), 2.80-2.60 (bs, 2H), 2.18-2.11 (m, 3H), 2.00-1.70 (m, 6H).

LCMS: 96.85%, m/z=411.2 (M+1). HPLC: 95.08%.

### Example 7

(S)-6-(3-aminopyrrolidin-1-yl)—N—(2-methyl-5-(piperidin-1-yl)-2H-indazol-6-yl)picolinamide

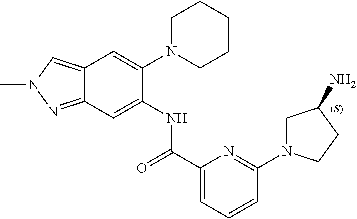

In a sealed tube, 6-bromo-N-(2-methyl-5-(piperidin-1-yl)-2H-indazol-6-yl)picolinamide (product of step 1 of example 3), tert-butyl (R)-pyrrolidin-3-ylcarbamate (203 mg, 1.08 mmol), sodium carbonate (307 mg, 2.89 mmol) and DMF (6 mL) were taken and heated at 140° C. for 4h. The reaction was quenched with ice water and extracted with ethyl acetate. The organic layer was dried over Na₂SO₄ and concentrated under reduced pressure to obtain the desired product. The crude product was dissolved in DCM (10 mL) to this solution TFA/DCM (1/1 mL) was added and stirred at room temperature for 3 h. After completion of reaction, excess of solvent was removed under reduced pressure, basified with saturated sodium carbonate solution and diluted with ethyl acetate. The organic layer was washed with brine and dried over anhydrous Na₂SO₄. After concentration, the residue was purified by column chromatography (CH₂Cl₂:MeOH; 98:2) to obtain the title compound (40 mg, 35.3%).

¹HNMR (CDCl₃, 400 MHz): δ 10.90 (s, 1H), 7.76 (s, 1H), 7.65-7.64 (m, 2H), 7.32 (s, 1H), 6.56-6.54 (m, 1H), 4.19 (s, 3H), 3.89-3.87 (m, 1H), 3.82-3.79 (t, 2H), 3.70-3.65 (m, 2H), 3.51-3.49 (m, 1H), 3.40-3.39 (m, 2H), 2.29-2.28 (m, 2H), 1.90-1.75 (m, 7H).

LCMS: 98.52%, m/z=420.3 (M+1). HPLC: 97.46%.

### Example 8

(S)-6-(3-aminopyrrolidin-1-yl)—N—(1-methyl-5-(piperidin-1-yl)-1H-indazol-6-yl)picolinamide

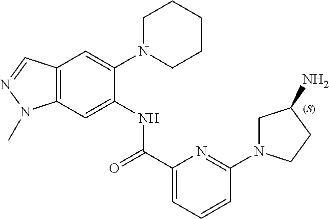

### Step-1: Preparation of 6-bromo-N-(1-methyl-5-(piperidin-1-yl)-1H-indazol-6-yl)picolinamide

The title compound was prepared according to the procedure described in step-1 of Example 3 by using the appropriate reactants and reaction conditions. Yield: 700 mg (90.4%).

LCMS: 95.68%, m/z=414.1 (M+1)

### Step-2: Preparation of (S)-6-(3-aminopyrrolidin-1-yl)—N—(1-methyl-5-(piperidin-1-yl)-1H-indazol-6-yl)picolinamide

The title compound was prepared according to the procedure described in product of step-2 of example 3 and example 4 by using the appropriate reactants and reaction conditions. 6-bromo-N-(1-methyl-5-(piperidin-1-yl)-2H-indazol-6-yl)picolinamide (400 mg, 0.966 mmol), tert-butyl (S)-pyrrolidin-3-ylcarbamate (270 mg, 1.44 mmol), sodium carbonate (409 mg, 3.86 mmol) and DMF (6 mL) and heated at 140° C. for 4h. Reaction was quenched with ice water and extracted with ethyl acetate dried over Na₂SO₄ concentrated under reduced pressure to get the crude product (140 mg, 28%). The crude product was dissolved in DCM (10 mL) to this solution TFA/DCM (1/1 mL) was added and stirred at room temperature for 3h to obtain the title compound (50 mg, 44.24%).

¹HNMR (DMSO-d₆, 400 MHz): δ 10.95 (s, 1H), 8.67 (s, 1H), 7.96-7.83 (m, 3H), 7.83-7.79 (t, 1H), 7.62 (s, 1H), 7.53-7.51 (d, 1H), 6.85-6.83 (d, 1H), 4.03 (s, 1H), 3.99 (s, 3H), 3.83-3.69 (m, 3H), 2.85 (s, 4H), 2.12-2.08 (m, 1H), 1.76-1.75 (m, 4H), 1.59 (bs, 2H). LCMS: 95.4%, m/z=420.2 (M+1). HPLC: 96.29%.

### Example 9

(S)-6-(3-hydroxypyrrolidin-1-yl)—N—(1-methyl-5-(piperidin-1-yl)-1H-indazol-6-yl)picolinamide

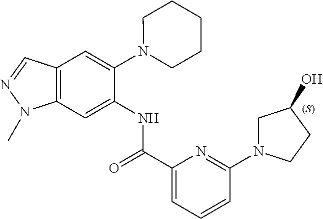

The title compound was prepared according to the procedure described in product of step-1 and step-2 of example 3 by using the appropriate reactants and reaction conditions. Yield: 50 mg (50%).

¹HNMR (CDCl₃, 400 MHz): δ 11.1 (s, 1H), 8.79 (s, 1H), 7.87 (s, 1H), 7.70-7.64 (m, 2H), 7.49 (s, 1H), 6.62-6.59 (dd, 1H), 4.70 (s, 1H), 4.08 (s, 3H), 3.84-3.79 (m, 4H), 3.20-2.70 (bs, 4H), 2.29-2.17 (m, 3H), 1.90-1.80 (m, 6H). LCMS: 99.0%, m/z=421.5 (M+1). HPLC: 97.08%.

### Example 10

(S)-2-(3-hydroxypyrrolidin-1-yl)—N—(1-methyl-5-(piperidin-1-yl)-1H-indazol-6-yl)oxazole-4-carboxamide

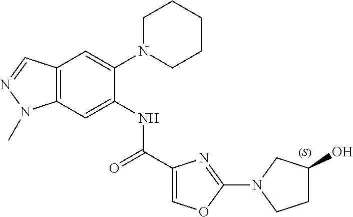

The title compound was prepared according to the procedure described in product of example 6 by using the appropriate reactants and reaction conditions. Yield: 40 mg (33.3%)

¹HNMR (CDCl₃, 300 MHz): δ 10.52 (s, 1H), 8.63 (s, 1H), 7.85 (s, 1H), 7.83 (s, 1H), 7.43 (s, 1H), 4.65 (s, 1H), 4.04 (s, 3H), 3.76-3.61 (m, 4H), 3.10-2.90 (bs, 2H), 2.80-2.60 (bs, 2H), 2.25-2.10 (m, 3H), 2.0-1.70 (m, 6H). HPLC: 96.45%.

### Example 11

(S)—N—(5-(3-hydroxypyrrolidin-1-yl)-1-methyl-1H-indazol-6-yl)-2-(2-methylpyridin-4-yl)oxazole-4-carboxamide hydrochloride

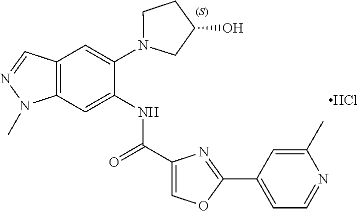

### Step-1: Preparation of 4-fluoro-2-methyl-5-nitroaniline

A solution of 4-fluoro-2-methylaniline (12 g, 96 mmol) in Con.H₂SO₄ (110 ml) was cooled to 0° C. and added KNO₃ (10.6 g, 105.6 mmol) and stirred at room temperature for 1h. The reaction mass was diluted by water and basified with 20% NaOH. The compound was extracted with ethyl acetate, dried over Na₂SO₄ and concentrated to obtain the title compound (15 g, 81.9%.)

¹HNMR (CDCl₃, 300 MHz): δ 7.61 (d, 1H), 7.34 (d, 1H), 2.24 (s, 3H).

### Step-2: Preparation of (S)-1-(4-amino-5-methyl-2-nitrophenyl)pyrrolidin-3-ol.

A solution of 4-fluoro-2-methyl-5-nitroaniline (11 g, 64.32 mmol), potassium carbonate (35.5 g, 257.30 mmol) and (S)-pyrrolidin-3-ol (8.7 g, 70.76 mmol) in THF was stirred at 70° C. for 12h. The reaction mixture was filtered and filtrate was purified by column chromatography elution with 50% ethyl acetate in hexane to obtain the title compound (11 g, 72.3%.). LCMS: 97.15% m/z=238.3(M+1).

### Step-3: Preparation of (S)-1-(6-nitro-1H-indazol-5-yl) pyrrolidin-3-ol.

A solution of (S)-1-(4-amino-5-methyl-2-nitrophenyl)pyrrolidin-3-ol (2 g, 8.43 mmol) (product of step-2 of example 11) in chloroform (50 ml) was added potassium acetate (992 mg, 10.12 mmol), acetic anhydride (2.58 g, 25.314 mmol) and stirred at 40° C. for 30 min. Isoamylnitrite (1.98 g, 16.87 mmol) was added at 40° C. heated to 60° C. for 12h. The reaction mass was basified up to pH-9. The compound was extracted by using sodium bicarbonate solution with chloroform, dried over Na₂SO₄, and concentrated and purified by column chromatography elution with 2% methanol in dichloromethane. This was then treated with methanolic HCl to obtain the title compound (480 mg, 20.2%).

¹HNMR (CDCl₃, 300 MHz): δ 8.02 (s,1H), 7.88 (s, 1H),7.22 (s, 1H),4.65-4.60 (m, 1H), 3.57-3.51 (m, 4H) 3.26-3.21 (m, 1H), 2.98(d, 1H), 2.22-2.05 (m, 4H); LCMS:71.1%, m/z=249.15(M+1).

### Step-4: Preparation of (S)-5-(3-((tert-butyldimethylsily)oxy)pyrrolidin-1-yl)-6-nitro-1H-indazole

To the solution of (S)-1-(6-nitro-1H-indazol-5-yl) pyrrolidin-3-ol (product of step-3 of example 11) (650 mg, 2.33 mmol) in DMF (10 mL) DMAP (319 mg, 2.62 mmol), TBDMS chloride (790 mg, 5.24 mmol) and imidazole (267 mg, 3.930 mmol) were added and stirred at RT for 2h to get the crude product. Purification was done by 60-120 silica gel column chromatography using 20% ethyl acetate in hexane as eluent to obtain the title compound (680 mg, 78%). LCMS: 70.9% m/z=363.15(M+1).

### Step-5: Preparation of (S)-5-(3-((tert-butyldimethylsilyl) oxy)pyrrolidin-1-yl)-2-methyl-6-nitro-2H-indazole and (S)-5-(3-((tert-butyldimethylsilyl)oxy)pyrrolidin-1-yl)-1-methyl-6-nitro-1H-indazole

To a solution of sodium hydride (255 mg, 5.313 mmol) in THF (50 mL) was added (S)-5-(3-((tert-butyldimethylsilyl)oxy)pyrrolidin-1-yl)-6-nitro-1H-indazole (product of step-4 of example 11) (1.3 g, 3.54 mmol) at 0° C. After 15 min to that solution at 0° C. methyl iodide (1.01 g, 7.084 mmol) was added. The reaction mixture was allowed to room temperature for 2h. The reaction mixture was diluted with EtOAc, washed with brine and dried over anhydrous Na₂SO₄. This was purified by silica gel column chromatography and elution with 20% ethyl acetate in hexane gave isomer A: (S)-5-(3-((tert-butyldimethylsilyl)oxy)pyrrolidin-1-yl)-2-methyl-6-nitro-2H-indazole (700 mg, 48.2%). Elution with 50% ethyl acetate in hexane gave isomer B: S)-5-(3-((tert-butyldimethylsilyl)oxy)pyrrolidin-1-yl)-1-methyl-6-nitro-1H-indazole (500 mg, 37.5%).

¹HNMR (CDCl₃, 300 MHz): δ 7.90 (s, 1H) 7.80 (s, 1H) 7.17 (s, 1H) 4.5 (m, 1H) 4.06 (s, 3H) 3.50-3.30 (m, 3H) 2.90-2.83 (m, 1H) 2.12-1.96 (m, 2H) 0.85 (s, 9H) 0.1 (s, 6H) LCMS:93.36% m/z=377.20(M+1).

### Step-6: (S)-5-(3-((tert-butyldimethylsilyl)oxy)pyrrolidin-1-yl)-1-methyl-1H-indazol-6-amine

To a solution of (S)-5-(3 -((tert-butyldimethylsilyl)oxy)pyrrolidin-1-yl)-1-methyl-6-nitro-1H-indazole (product of step-5 isomer-B of example 11) (500 mg, 1.32 mmol) in THF (20 mL) ammonium chloride (1.15 g, 21.20 mmol) in water (5 mL) was added and zinc dust (691 mg, 10.63 mmol) and stirred at RT for 30 min. The catalyst was filtered through Celite®, the compound was extracted with ethyl acetate and distilled out the solvent to obtain the title compound (450 mg, 97.8%). LCMS: 88.2% m/z=347.25(M+1).

### Step-7: Preparation of (S)—N—(5-(3-hydroxypyrrolidin-1-yl)-1-methyl-1H-indazol-6-yl)-2-(2-methylpyridin-4-yl)oxazole-4-carboxamide hydrochloride

To a solution of (S)-5-(3-((tert-butyldimethylsilyl)oxy)pyrrolidin-1-yl)-1-methyl-1H-indazol-6-amine (product of step-6 of example 11) (200 mg, 0. 576 mmol) in DMF (8 mL) 2-(2-methylpyridin-4-yl)oxazole-4-carboxylic acid (118 mg, 0. 576 mmol), HATU (328 mg, 864 mmol), DIPEA (297 mg, 2.304 mmol) were added . The reaction mixture was stirred for 12 h at room temperature. After completion of reaction, reaction mixture was diluted with EtOAc, washed with brine and dried over anhydrous Na₂SO₄. This was then treated with methanolic HCl to obtain the title compound (120 mg, 61.2%).

¹HNMR (CDCl₃, 400 MHz): δ 10.6 (s, 1H), 8.61 (s, 1H), 8.43 (s, 1H), 7.88 (d, 2H), 7.78 (d, 1H), 7.57 (s, 1H), 4.62 (bs, 1H), 4.09 (s, 3H), 3.44-3.41 (m,1H), 3.24 (d, 1H), 3.16-3.12 (m, 1H), 3.04-2.97 (m, 1H), 2.68 (s, 3H), 2.62-2.52 (m, 2H), 2.17-2.12 (m, 1H). LCMS: 100%, m/z=419.1 (M+1). HPLC: 97.12%.

### Example 12

N—(5-(3-hydroxypiperidin-1-yl)-1-methyl-1H-indazol-6-yl)-2-(2-methylpyridin-4-yl)oxazole-4-carboxamide hydrochloride

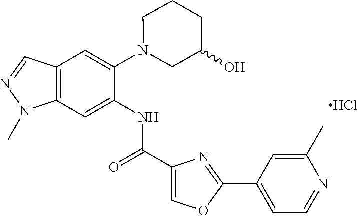 [SMILES: Cc1cc(-c2nc(C(=O)Nc3cc4c(cnn4C)cc3N3CCCC(O)C3)co2)ccn1 ; Cl]

The title compound was prepared according to the procedure described in product of steps 1 to 7 of example 11 by using 5-(3-((tert-butyldimethylsilyl)oxy)piperidin-1-yl)-1-methyl-1H-indazol-6-amine (150 mg, 0. 416 mmol) and 2-(2-methylpyridin-4-yl)oxazole-4-carboxylic acid under same reaction conditions. Yield: 65 mg (58.23%)

¹HNMR (DMSO-d₆, 400 MHz): δ 10.7 (s, 1H), 9.05 (s, 1H), 8.73 (d, 1H), 8.54 (s, 1H), 7.94 (s, 1H), 7.88 (s, 1H), 7.77 (d, 1H), 7.67 (s, 1H), 4.93 (bs, 1H), 4.08 (s, 3H), 3.06 (d, 1H), 2.86-2.85 (m, 1H), 2.71-2.70 (m, 1H), 2.61 (s, 3H), 2.08-2.07 (m, 2H), 1.93-1.90 (m, 2H), 1.45-1.35 (m, 2H). LCMS: 100%, m/z=433.1 (M+1). HPLC: 97.59%

### Example 13

N—(5-(3-hydroxypiperidin-1-yl)-2-methyl-2H-indazol-6-yl)-2-(2-methylpyridin-4-yl)oxazole-4-carboxamide hydrochloride

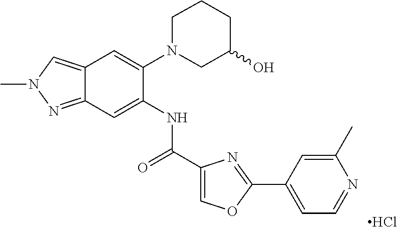

### Step-1: Preparation of 5-(3-((tert-butyldimethylsily)oxy)piperidin-1-yl)-2-methyl-2H-indazol-6-amine

The title compound was prepared according to the procedure described in step-6 of example 11 by using 5-(3-((tert-butyldimethylsily)oxy)piperidin-1-yl)-2-methyl-6-nitro-2H-indazole (300 mg, 0.769 mmol) under same reaction conditions. Yield: 200 mg (73.52%); LCMS: 84.2%, m/z=361.41 (M+1)

### Step-2: Preparation of N—(5-(3-hydroxypiperidin-1-yl)-2-methyl-2H-indazol-6-yl)-2-(2-methylpyridin-4-yl)oxazole-4-carboxamide hydrochloride

Using the same reaction conditions as described in step-7 of example 11, 5-(3-((tert-butyldimethylsilyl)oxy)piperidin-1-yl)-2-methyl-2H-indazol-6-amine (170 mg, 0.471 mmol) in

DMF (8 mL) 2-(2-methylpyridin-4-yl)oxazole-4-carboxylic acid (105 mg, 0.518 mmol), HATU(268 mg, 0.070 mmol), DIPEA (243 mg, 1.8 mmol) were added. The reaction mixture was stirred for 12 h at room temperature. The reaction mass was quenched by ice and the solid was filtered. This was then treated with methanolic. HCl to obtain the title compound (140 mg, 86.5%).

¹HNMR (DMSO-d₆, 400 MHz): δ 10.7 (s, 1H), 9.05 (s, 1H), 8.73 (d, 1H), 8.55 (s, 1H), 8.22 (s, 1H), 7.88 (s, 1H), 7.77 (d, 1H), 7.54 (s, 1H), 4.93 (bs, 1H), 4.12 (s, 3H), 4.00 (s, 1H), 3.08-3.07 (m, 2H), 2.40-2.35 (m, 1H), 2.67-2.61 (m, 1H), 2.61 (s, 3H), 2.18-2.08 (m, 2H), 1.95-1.90 (m, 2H). LCMS: 100%, m/z=433.1 (M+1). HPLC: 95.20%.

### Example 14

N—(5-(3-fluoropiperidin-1-yl)-2-methyl-2H-indazol-6-yl)-2-(2-methylpyridin-4-yl)oxazole-4-carboxamide

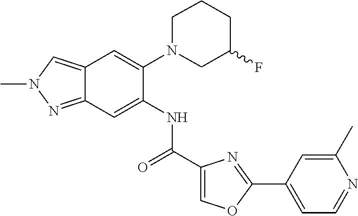

### Step-1: Preparation 1-(5-(3-hydroxypiperidin-1-yl)-6-nitro-1H-indazol-1-yl)ethan-1-one

Using the same reaction conditions as described in step-2 of example 11, 1-(4-amino-5-methyl-2-nitrophenyl)piperidin-3-ol (8 g, 31.8 mmol) in chloroform (100 ml) potassium acetate (3.7 g, 38.0 mmol), acetic anhydride (9.75 g, 96.0 mmol) were added and stirred the reaction mixture at 40° C. for 30 min. Then, isoamylnitrite (7.45 g, 63 mmol) was added at 40° C. and heated to 60° C. for 12h. The reaction mass was basified to pH-9 using sodiumbicabonate solution. The compound was extracted with chloroform, dried over Na₂SO₄ and concentrated to get crude product which was purified by column chromatography using 2% methanol in dichloromethane as eluent to obtain the title compound (4 g, 41.0%). LCMS: 76.4%, m/z=305.3 (M-1).

### Step-2: Preparation 5-(3-fluoropiperidin-1-yl)-6-nitro-1H-indazole

To a solution of 1-(5-(3-hydroxypiperidin-1-yl)-6-nitro-1H-indazol-1-yl)ethan-1-one (product of step-1 of example 14) (3.7 g, 12.0 mmol) in dichloromethane (30 ml) was cooled to −70° C., and DAST (3.3 g, 20.0 mmol) in dichloromethane(10 ml) was added and the reaction mixture was stirred at −50° C. for 2h. The reaction mass was quenched by NaHCO₃ solution and the compound was extracted with DCM, dried over Na₂SO₄. The excess solvent was evaporated under reduced pressure and the compound was purified by column chromatography using 10% of EtOAc in hexane. This was then treated with methanolic HCl to obtain the title compound (1.4 g, 63.6%). LCMS: 81.4%, m/z=307.15 (M−1).

### Step-3: Preparation of 5-(3-fluoropiperidin-1-yl)-1-methyl-6-nitro-1H-indazole and 5-(3-fluoropiperidin-1-yl)-2-methyl-6-nitro-2H-indazole

The title compound was prepared according to the procedure described in step-5 example 11 by using 5-(3-fluoropiperidin-1-yl)-6-nitro-1H-indazole (product of step-2 of example 14) under the same reaction conditions. Yield: 400 mg (31.7%).

¹HNMR (CDCl₃, 300 MHz): δ 8.02 (s, 1H) 7.89 (s, 1H) 7.37 (s, 1H) 4.72-4.60 (m, 1H) 4.23 (s, 1H) 3.50-3.40 (m, 1H) 3.10-3.00 (m, 1H) 2.95-2.85 (m, 1H) 2.75-2.65 (m, 1H) 2.20-2.10 (m, 1H) 1.95-1.85 (m, 1H) 1.25-1.10 (m, 2H). LCMS: 99.25%, m/z=279.1 (M+1).

### Step-4: Preparation of 5-(3-fluoropiperidin-1-yl)-2-methyl-2H-indazol-6-amine

The title compound was prepared according to the procedure described in step-6 example 11 by using 5-(3-fluoropiperidin-1-yl)-2-methyl-6-nitro-2H-indazole (product of step-3 isomer-B of example 14) (400 mg, 1.4 mmol) under the same reaction conditions. Yield: 300 mg (83.8%); LCMS: 85.88%, m/z=249.3 (M+1).

### Step-5: Preparation of N—(5-(3-fluoropiperidin-1-yl)-2-methyl-2H-indazol-6-yl)-2-(2-methylpyridin-4-yl)oxazole-4-carboxamide

Using the same reaction conditions as described in step-7 of example 11, 5-(3-fluoropiperidin-1-yl)-2-methyl-2H-indazol-6-amine (100 mg, 0.400 mmol) in DMF (8 mL), was reacted with 2-(2-methylpyridin-4-yl)oxazole-4-carboxylic acid (98 mg, 0.48 mmol), to obtain the title compound (50 mg, 28.4%).

¹HNMR (CDCl₃, 400 MHz): δ 10.8 (bs, 1H), 8.87 (s, 1H), 8.69 (d, 1H), 8.41 (s, 1H), 8.0-7.8 (m, 3H), 7.38 (s, 1H), 5.0 (d, 1H), 4.20 (s, 3H), 3.50-3.49 (m, 1H), 3.20-3.00 (m, 2H), 2.68 (s, 3H), 2.55-2.45 (m, 1H), 2.10-2.0 (m, 2H), 1.80-1.70 (m, 2H). LCMS: 99.35%, m/z=435.3 (M+1). HPLC: 99.42%.

### Example 15

((S)-2-(2-acetamidopyridin-4-yl)—N—(5-(3-hydroxypyrrolidin-1-yl)-1-methyl-1H-indazol-6-yl)oxazole-4-carboxamide

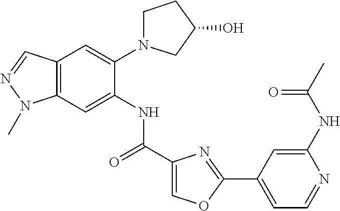 [SMILES: CC(=O)Nc1cc(-c2nc(C(=O)Nc3cc4c(cnn4C)cc3N3CC[C@H](O)C3)co2)ccn1]

Using the same reaction conditions as described in step-7 of example 11, (S)-5-(3-((tert-butyldimethylsilyl)oxy)pyrrolidin-1-yl)-1-methyl-1H-indazol-6-amine (product of step-6 of example 11) (200 mg, 0.576 mmol) was reacted with 2-(2-acetamidopyridin-4-yl)oxazole-4-carboxylic acid (intermediate 16) (180 mg, 0.749 mmol) to obtain the title compound (120 mg, 93.7%).

¹HNMR (CDCl₃, 300 MHz): δ 10.6 (s, 1H), 8.91 (s, 1H), 8.66 (s, 1H), 8.45-8.40 (m, 2H), 8.15 (s, 1H), 7.87(s, 1H), 7.67 (d, 1H), 7.54 (s, 1H), 4.76 (bs, 1H), 4.08 (s, 3H), 3.78 (s, 1H), 3.55-3.45 (m, 1H), 3.40-3.30 (m, 1H), 3.30-3.20 (m, 2H), 3.00-2.90 (m, 1H), 2.60-2.50 (m, 1H), 2.28 (s, 3H). LCMS: 94.78%, m/z=462.20 (M+1). HPLC: 95.02%.

### Example 16

N—(5-(3-fluoropiperidin-1-yl)-1-methyl-1H-indazol-6-yl)-2-(2-methylpyridin-4-yl)oxazole-4-carboxamide

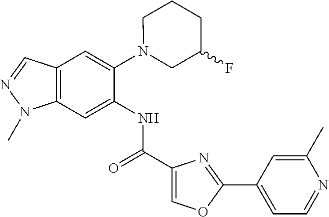 [SMILES: Cc1cc(-c2nc(C(=O)Nc3cc4c(cnn4C)cc3N3CCCC(F)C3)co2)ccn1]

### Step-1: Preparation of 5-(3-fluoropiperidin-1-yl)-1-methyl-1H-indazol-6-amine

The title compound was prepared according to the procedure described in step-6 example 11 by using 5-(3-fluoropiperidin-1-yl)-1-methyl-6-nitro-1H-indazole (product of step-3 isomer-A of example 14) (400 mg, 1.4 mmol) under the same reaction conditions. Yield: 300 mg (83.8%); LCMS: 85.88%, m/z=249.3 (M+1).

### Step-2: Preparation of N—(5-(3-fluoropiperidin-1-yl)-1-methyl-1H-indazol-6-yl)-2-(2-methylpyridin-4-yl)oxazole-4-carboxamide

Using the same reaction conditions as described in step-7 of example 11, 5-(3-fluoropiperidin-1-yl)-1-methyl-1H-indazol-6-amine (100 mg, 0.400 mmol) was reacted with 2-(2-methylpyridin-4-yl)oxazole-4-carboxylic acid to obtain the title compound (50 mg, 28.5%).

¹HNMR (CDCl₃, 400 MHz): δ 10.9 (bs, 1H), 8.68 (s, 2H), 8.40 (s, 1H), 7.96-7.70 (m, 3H), 7.51 (s, 1H), 5.0 (d, 1H), 4.08 (s, 3H), 3.50-3.30 (m, 1H), 3.20-3.00 (m, 2H), 2.90-2.70 (m, 1H), 2.68 (s, 3H), 2.50-2.30 (m, 2H), 2.15-1.95 (m, 1H), 1.85 1.70 (m, 1H).

LCMS: 100%, m/z=436.0 (M+1). HPLC: 98.45%.

### Example 17

N—(5-(4-hydroxypiperidin-1-yl)-1-methyl-1H-indazol-6-yl)-2-(2-methylpyridin-4-yl)oxazole-4-carboxamide

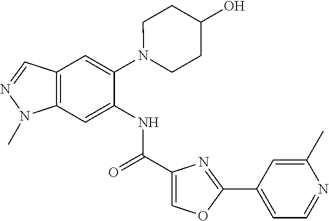 [SMILES: Cc1cc(-c2nc(C(=O)Nc3cc4c(cnn4C)cc3N3CCC(O)CC3)co2)ccn1]

The title compound was prepared according to the procedure described in steps-2 to 7 of example 11 by reacting 5-(4-((tert-butyldimethylsilyl)oxy)piperidin-1-yl)-1-methyl-1H-indazol-6-amine (90 mg, 0.26 mmol) with 2-(2-methylpyridin-4-yl)oxazole-4-carboxylic acid (64 mg, 0.312 mmol) under the same reaction conditions. Yield: 55 mg (83%).

¹HNMR (DMSO-d₆, 300 MHz): δ 10.80 (s, 1H), 9.06 (s, 1H), 8.66 (d, 1H), 8.51 (s, 1H), 7.80 (d, 2H), 7.78 (d, 1H), 7.64 (s, 1H), 4.97 (bs, 1H), 3.98 (s, 3H), 3.80-3.70 (m, 1H), 3.05-2.95 (m, 2H), 2.90-2.80 (m, 3H), 2.60 (s, 2H), 2.10-1.85 (m, 4H). LCMS: 100%, m/z=433.7 (M+1). HPLC: 96.73%.

### Example 18

(S)-2-(2-aminopyridin-4-yl)—N—(5-(3-hydroxypyrrolidin-1-yl)-1-methyl-1H-indazol-6-yl)oxazole-4-carboxamide

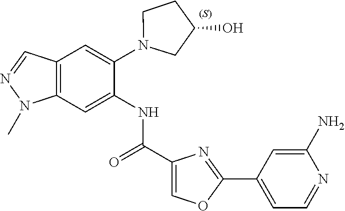

To a solution of (S)-2-(2-acetamidopyridin-4-yl)—N—(5-(3-hydroxypyrrolidin-1-yl)-1-methyl-1H-indazol-6-yl)oxazole-4-carboxamide (Product of step-1 of example 15) (90 mg, 0.194 mmol) in methanol (3 ml) Con. HCl (1 ml) was added and stirred at 65° C. for 30 mins. The solvent was distilled out and purified by preparative HPLC to obtain the title compound (15 mg, 18.5%).

¹HNMR (CDCl₃, 300 MHz): δ 10.6 (s, 1H), 8.61 (S, 1H), 8.39 (s, 1H), 8.25 (d, 1H), 7.87 (d, 1H), 7.56 (s, 1H), 7.28 (d, 2H), 4.75-4.60 (m, 3H), 4.08 (s, 3H), 3.50-3.40 (m, 1H), 3.30-3.20 (m, 2H), 3.00-2.90 (m, 2H), 2.60-2.50 (m, 1H), 2.30-2.20 (m, 1H). LCMS: 100%, m/z=420.0 (M+1). HPLC: 95.56%.

### Example 19

N—(5-(4-fluoropiperidin-1-yl)-1-methyl-1H-indazol-6-yl)-2-(2-methylpyridin-4-yl)oxazole-4-carboxamide

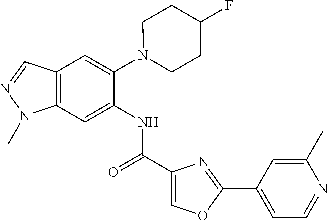 [SMILES: Cc1cc(-c2nc(C(=O)Nc3cc4c(cnn4C)cc3N3CCC(F)CC3)co2)ccn1]

The title compound was prepared according to the procedure described in product of step-1 to 5 of example 14 by reacting 5-(4-fluoropiperidin-1-yl)-1-methyl-1H-indazol-6-amine (200 mg, 0.80 mmol) with 2-(2-methylpyridin-4-yl)oxazole-4-carboxylic acid (197 mg, 0.90 mmol) under the same reaction conditions. Yield: 50 mg (28.4%).

¹HNMR (DMSO-d₆, 400 MHz): δ 10.6 (s, 1H), 9.11 (s, 1H), 9.70 (d, 1H), 8.55 (s, 1H), 7.95 (s, 1H), 7.86 (s, 1H), 7.60 (d, 1H), 7.70 (s, 1H), 5.05-4.90 (m, 1H), 4.01 (s, 3H), 3.10-3.00 (m, 2H), 2.93-2.89 (m, 2H), 2.59 (s, 3H), 2.30-2.10 (m, 4H). LCMS: 96.6%, m/z=435.3 (M+1). HPLC: 97.9%.

### Example 20

N-(5-(4-(hydroxymethyl) piperidin-1-yl)-1-methyl-1H-indazol-6-yl)-2-(2-methylpyridin-4-yl)oxazole-4-carboxamide

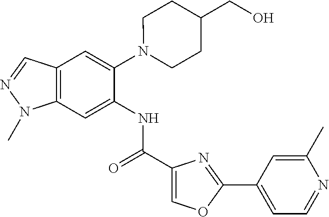 [SMILES: Cc1cc(-c2nc(C(=O)Nc3cc4c(cnn4C)cc3N3CCC(CO)CC3)co2)ccn1]

The title compound was prepared according to the procedure described in step 1 to step 7 of example 11 by reacting 5-(4-(((tert-butyldimethylsilyl)oxy)methyl)piperidin-1-yl)-1-methyl-1H-indazol-6amine(150 mg, 0.403 mmol) (product of step-5 of example 20) with 2-(2-methylpyridin-4-yl)oxazole-4-carboxylic acid (82 mg, 0.403 mmol) under the same reaction conditions. Yield: 40 mg (23.5%).

¹HNMR (CDCl₃, 400 MHz): δ 10.60 (s, 1H), 8.70 (d, 1H), 8.66 (s, 1H), 8.43 (s, 1H), 7.88 (s, 1H), 7.82 (s, 1H), 7.80 (d, 1H), 7.52 (s, 1H), 4.08 (s, 3H), 3.72 (s, 2H), 3.16 (d, 2H), 2.83 (t, 2H), 2.70 (s, 3H), 2.05-1.95 (m, 2H), 1.85-1.75 (m, 3H), 1.52-1.48 (m, 1H). LCMS: 98.77%, m/z=447.4 (M+1). HPLC: 96.07%.

### Example 21

(S)-2-(2,6-dimethylpyridin-4-yl)—N—(5-(3-hydroxypyrrolidin-1-yl)-1-methyl-1H-indazol-6-yl)oxazole-4-carboxamide

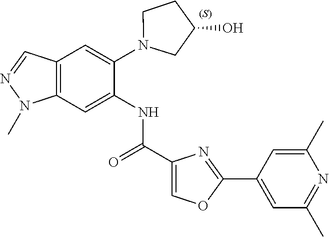

Using the same reaction conditions as described in step-7 of example 11, (S)-5-(3-((tert-butyldimethylsilyl)oxy)pyrrolidin-1-yl)-1-methyl-1H-indazol-6-amine (product of step-6 of example 11) in DMF (8 mL) was reacted with 2-(2,6-dimethylpyridin-4-yl)oxazole-4-carboxylic acid (intermediate 11) to obtain the title compound (80 mg, 88.8%).

¹HNMR (DMSO-d₆, 400 MHz): δ 10.43 (s, 1H), 9.02 (s, 1H), 8.45 (s, 1H), 7.88 (s, 1H), 7.68 (s, 2H), 7.63 (s, 1H), 5.08 (d, 1H), 4.51 (bs, 1H), 3.96 (s, 3H), 3.30-3.19 (m, 1H), 2.96-2.92 (m, 2H), 2.46 (s, 6H), 2.35-2.25 (m, 1H), 2.00-1.85 (m, 1H). LCMS: 100%, m/z=433.1 (M+1). HPLC: 98.64%.

### Example 22

(R)—N—(5-(3-hydroxypyrrolidin-1-yl)-1-methyl-1H-indazol-6-yl)-2-(2-methylpyridin-4-yl)oxazole-4-carboxamide

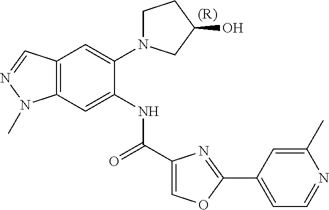

The title compound was prepared according to the procedure described in step 1 to step 7 of example 11 by using the appropriate reactants and reaction conditions. Yield: 60 mg (33.3%).

¹HNMR (DMSO-d₆, 400 MHz): δ 10.41 (S, 1H), 9.07 (s, 1H), 8.69 (d, 1H), 8.49 (s, 1H), 7.92 (d, 2H), 7.82 (d, 1H), 7.67 (s, 1H), 5.12 (bs, 1H), 4.54 (bs, 1H), 4.00 (s, 3H), 3.35-3.22 (m, 2H), 3.01-2.96 (m, 2H), 2.60 (s, 3H), 2.36-2.31 (m, 1H), 2.00-1.90 (m, 1H). LCMS: 100%, m/z=419.3 (M+1). HPLC: 95.60%

### Example 23

(S)-2-(2-aminopyridin-3-yl)—N—(5-(3-hydroxypyrrolidin-1-yl)-1-methyl-1H-indazol-6-yl)oxazole-4-carboxamide

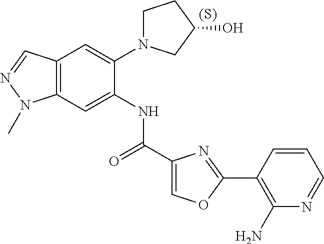

Using the procedure described in step-7 of example 11, (S)-5-(3-((tert-butyldimethylsilyl)oxy)pyrrolidin-1-yl)-1-methyl-1H-indazol-6-amine (product of step-6 of example 11) (120 mg, 0.485 mmol) was reacted with 2-(2-aminopyridin-3-yl)oxazole-4-carboxylic acid (intermediate 15) (168 mg, 0.534 mmol) to obtain the title compound (34 mg, 17%).

¹HNMR (DMSO-d₆, 400 MHz): δ 10.5 (bs, 1H), 9.10 (s, 1H), 8.90-8.70 (bs, 2H), 8.62 (d, 1H), 8.31 (d, 1H), 8.10-8.02 (m, 2H), 7.70-7.60 (bs, 1H), 7.08 (t, 1H), 4.50-4.40 (m, 2H), 4.02 (s, 3H), 3.60-3.45 (m, 2H), 3.20-3.10 (m, 1H), 2.24-2.18 (m, 1H), 1.95-1.85 (m, 1H), 1.21 (d, 1H). LCMS: 100%, m/z=420.2 (M+1). HPLC: 92.37%.

### Example 24

6-((S)-3-hydroxypyrrolidin-1-yl)—N—(5-((R)-3-hydroxypyrrolidin-1-yl)-1-methyl-1H-indazol-6-yl)picolinamide

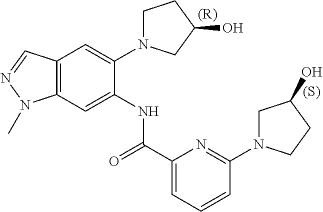

Using the procedure described in step-7 of Example 11, (R)-5-(3-((tert-butyldimethylsilyl)oxy)pyrrolidin-1-yl)-1-methyl-1H-indazol-6-amine (product of step-5 of example 22) (100 mg, 0.283 mmol) was reacted with (S)-6-(3-((tert-butyldimethylsilyl) oxy) pyrrolidin-1-yl) picolinic acid (intermediate 6) (110 mg, 0.34 mmol) to obtain the title compound (100 mg, 83.3%).

¹HNMR (DMSO-d₆, 300 MHz): δ 11.04 (s, 1H), 8.67 (s, 1H), 7.91 (s, 1H), 7.74 (t, 1H), 7.67 (s, 1H), 7.38 (d, 1H), 6.73 (d, 1H), 5.07 (bs, 1H), 4.45 (bs, 2H), 3.99 (s, 3H), 3.70-3.65 (m, 2H), 3.55-3.50 (m, 2H), 3.40-3.20 (m, 4H), 2.86 (dd, 1H), 2.20-2.15 (m, 2H), 2.00-1.80 (m, 2H) LCMS: 97.7%, m/z=423.4 (M+1). HPLC: 98.08%.

### Example 25

6-((S)-3-hydroxypyrrolidin-1-yl)—N—(5-((S)-3-hydroxypyrrolidin-1-yl)-1-methyl-1H-indazol-6-yl)picolinamide

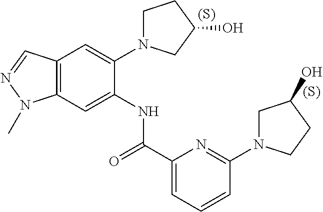

Using the procedure described in step-7 of Example 11, (S)-5-(3-((tert-butyldimethylsilyl)oxy)pyrrolidin-1-yl)-1-methyl-1H-indazol-6-amine (step-6 of example 11) (150 mg, 0.435 mmol) was reacted with (S)-6-(3-((tert-butyldimethylsilyl)oxy)pyrrolidin-1-yl) picolinic acid (intermediate 6) to obtain the desired compound (70 mg, 38.80%).

¹HNMR (DMSO-d₆, 400 MHz): δ 11.04 (s, 1H), 8.67 (s, 1H), 7.91 (s, 1H), 7.74 (t, 1H), 7.67 (s, 1H), 7.39 (d, 1H), 6.73 (d, 1H), 5.04 (bs, 1H), 4.45 (bs, 2H), 3.99 (s, 3H), 3.60-3.50 (m, 5H), 3.35-3.25 (m, 2H), 3.15-3.10 (m, 1H), 2.83 (dd, 1H), 2.20-2.15 (m, 2H), 2.00-1.80 (m, 2H) LCMS: 96.6%, m/z=423.4 (M+1). HPLC: 97.80%.

### Example 26

(S)—N—(5-(3-hydroxypyrrolidin-1-yl)-1-methyl-1H-indazol-6-yl)-2-(1H-pyrrolo[2,3-b]pyridin-4-yl)oxazole-4-carboxamide

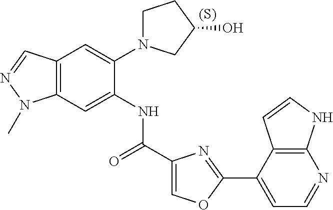

Using the procedure described in step-7 of Example 11, (S)-5-(3-((tert-butyldimethylsilyl)oxy)pyrrolidin-1-yl)-1-methyl-1H-indazol-6-amine (step-6 of example 11) (106 mg, 0.305 mmol) was reacted with 2-(1H-pyrrolo[2,3-b]pyridin-4-yl)oxazole-4-carboxylic acid (intermediate 17) (70 mg, 0.305 mmol) to obtain the desired compound (98 mg, 57.6%).

¹HNMR (DMSO-d₆, 300 MHz): δ 12.13 (s, 1H), 10.40 (s, 1H), 9.09 (s, 1H), 8.56 (s, 1H), 8.44 (d, 1H), 7.94 (s, 1H), 7.78-7.72 (m, 3H), 7.20 (s, 1H), 5.10 (d, 1H), 4.60-4.55 (m, 1H), 4.01 (s, 3H), 3.49 (q, 1H), 3.34-3.25 (m, 1H), 3.20-3.10 (m, 1H), 2.92 (dd, 1H), 2.40-2.25 (m, 1H), 2.00-1.90 (m, 1H); LCMS: 85.4%, m/z=443.9 (M+1). HPLC: 99.46%

### Example 27

(S)—N—(5-(3-hydroxypyrrolidin-1-yl)-1H-indazol-6-yl)-2-(2-methylpyridin-4-yl)oxazole-4-carboxamide

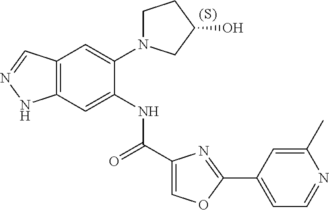

### Step-1: Preparation of (S)-5-(3-((tert-butyldimethylsily)oxy)pyrrolidin-1-yl)-1H-indazol-6-amine

To a solution of (S)-5-(3-((tert-butyldimethylsilyl)oxy)pyrrolidin-1-yl)-6-nitro-1H-indazole3 (product of step-3 Example 22) (2.1 g, 7.16 mmol) in THF (20 mL) ammonium chloride (6.13 g, 114 mmol) in water (5 mL) and zinc dust (3.74 g, 57.3 mmol) were added and stirred at RT for 30 min. The catalyst was filtered through Celite®. The Compound was extracted with ethyl acetate and the solvent was distilled out to obtain the title compound (1.92 g, 100%).) LCMS: 75.94%, m/z=333.30 (M+1)

### Step-2: Preparation of (S)—N—(5-(3-hydroxypyrrolidin-1-yl)-1H-indazol-6-yl)-2-(2-methylpyridin-4-yl)oxazole-4-carboxamide

Using the same reaction conditions as described in step-7 of example 11, (S)-5-(3-((tert-butyldimethylsilyl)oxy)pyrrolidin-1-yl)-1H-indazol-6-amine (step-1 of example 27) (150 mg, 0.451 mmol) was reacted with 2-(2-methylpyridin-4-yl)oxazole-4-carboxylic acid (101 mg, 0.496 mmol) to obtain the title compound (35 mg, 18.99%).

¹HNMR (DMSO-d₆, 400 MHz): δ 9.29 (s, 1H), 8.69 (d, 1H), 8.23 (s, 1H), 7.89 (s, 1H), 7.80 (d, 1H), 7.70 (s, 1H), 7.31 (s, 1H), 5.67 (d, 2H), 4.92 (d, 1H), 4.36-4.33 (m, 1H), 3.25-3.15 (m, 2H), 2.95-2.85 (m, 2H), 2.60 (s, 3H), 2.20-2.15 (m, 1H), 1.80-1.70 (m, 1H). LCMS: 100%, m/z=405.3 (M+1). HPLC: 95.19%.

### Example 28

(S)-2-(2-amino-3-fluoropyridin-4-yl)—N—(5-(3-hydroxypyrrolidin-1-yl)-1-methyl-1H-indazol-6-yl)oxazole-4-carboxamide

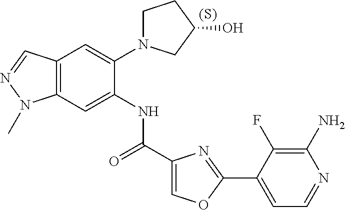

Using the same reaction conditions as described in step-7 of example 11, (S)-5-(3-((tert-butyldimethylsilyl)oxy)pyrrolidin-1-yl)-1-methyl-1H-indazol-6-amine (step-6 of example 11) (100 mg, 0.290 mmol) was reacted with 2-(2-amino-3-fluoropyridin-4-yl)oxazole-4-carboxylic acid (intermediate 14) (60 mg, 2.64 mmol) to obtain the title compound (11 mg, 9.11%).

¹HNMR (DMSO-d₆, 400 MHz): δ 10.32 (s, 1H), 9.08 (s, 1H), 8.50 (s, 1H), 7.91 (d, 1H), 7.67 (s, 1H), 7.16 (t, 1H), 6.65 (s, 1H), 5.03 (d, 1H), 4.55-4.50 (m, 1H), 4.00 (s, 3H), 3.17 (d, 2H), 3.05-3.00 (m, 1H), 2.92-2.88 (m, 1H), 2.32-2.28 (m, 1H), 1.95-1.85 (m, 1H). LCMS: 97.6%, m/z=438.1 (M+1). HPLC: 97.41%.

### Example 29

(R)-2-(2-aminopyridin-3-yl)—N—(5-(3-hydroxypyrrolidin-1-yl)-1-methyl-1H-indazol-6-yl)oxazole-4-carboxamide hydrochloride

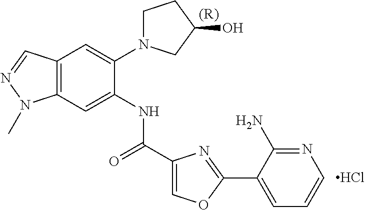

Using the same reaction conditions as described in step-7 of example 11, (R)-5-(3-((tert-butyldimethylsilyl)oxy)pyrrolidin-1-yl)-1-methyl-1H-indazol-6-amine (product of step 05 of example 22) (120 mg, 0.370 mmol) was reacted with 2-(2-aminopyridin-3-yl)oxazole-4-carboxylic acid (Intermediate 3) (80 mg, 0.322 mmol). This was further treated with methanolic HCl to obtain the title compound (10 mg, 21.2%).

¹HNMR (CDCl₃, 400 MHz): δ 8.60-8.50 (m, 2H), 8.25-8.15 (m, 1H), 7.97 (d, 2H), 7.65 (s, 1H), 6.94 (s, 1H), 4.05-3.95 (m, 2H), 3.55-3.45 (m, 1H), 3.25 (s, 6H), 2.40-2.30 (m, 1H), 2.20-2.10 (m, 1H). LCMS: 100%, m/z=420.3 (M+1). HPLC: 95.61%.

### Example 30

(S)—N—(5-(3-hydroxypyrrolidin-1-yl)-1-methyl-1H-indazol-6-yl)-2-(4-methylpiperazin-1-yl)oxazole-4-carboxamide hydrochloride

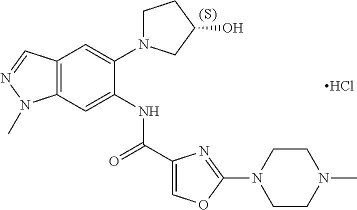

Using the same reaction conditions as described in step-7 of example 11, (S)-5-(3-((tert-butyldimethylsilyl)oxy)pyrrolidin-1-yl)-1-methyl-1H-indazol-6-amine (product of step 06 of example 11) (150 mg, 0.433 mmol) was reacted with 2-(4-methylpiperazin-1-yl)oxazole-4-carboxylic acid (Intermediate 3) (137 mg, 0.650 mmol). This was further treated with methanolic HCl to obtain the title compound (70 mg, 93.5%).

¹HNMR (CDCl₃, 300 MHz): δ 10.2 (s, 1H), 7.85-7.84 (m, 2H), 7.49 (s, 1H), 4.5 (bs, 1H), 4.05 (s, 3H), 3.60-3.57 (m, 4H), 3.40-3.30 (m, 1H), 3.16 (d, 1H), 3.03-2.92 (m, 2H), 2.70 (d, 1H), 2.51 (t, 4H), 2.50-2.40 (m, 2H), 2.34 (s, 3H), 2.05-1.95 (m, 1H). LCMS: 100%, m/z=427.0 (M+1). HPLC: 98.83%.

### Example 31

(S)—N—(5-(3-hydroxypyrrolidin-1-yl)-1-methyl-1H-indazol-6-yl)-2-(piperazin-1-yl)oxazole-4-carboxamide hydrochloride

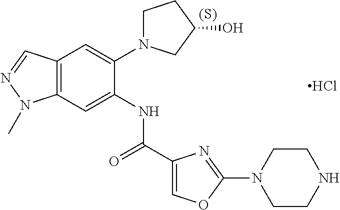

Using the same reaction conditions as described in step-7 of example 11, (S)-5-(3-((tert-butyldimethylsilyl)oxy)pyrrolidin-1-yl)-1-methyl-1H-indazol-6-amine (product of step-6 of example 11) (150 mg, 0.433 mmol) was reacted with 2-(4-(tert-butoxycarbonyl)piperazin-1-yl)oxazole-4-carboxylic acid (Intermediate 7) (142 mg, 0.650 mmol). This was further treated with methanolic HCl to obtain the title compound (25 mg, 54.5%).

¹HNMR (CDCl₃, 300 MHz): δ 10.2 (s, 1H), 8.56 (s, 1H), 7.86 (d, 2H), 7.49 (s, 1H), 4.5 (bs, 1H), 4.05 (s, 3H), 3.60-3.50 (m, 4H), 3.40-3.30 (m, 1H), 3.20 (d, 1H), 3.03-2.92 (m, 6H), 2.50-2.40 (m, 1H), 2.10-1.95 (m, 2H). LCMS: 98.6%, m/z=412.6 (M+1). HPLC: 96.01%.

### Example 32

(S)—N—(1-ethyl-5-(3-hydroxypyrrolidin-1-yl)-1H-indazol-6-yl)-2-(2-methylpyridin-4-yl)oxazole-4-carboxamide hydrochloride

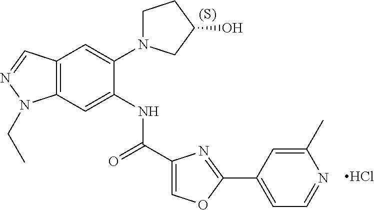

### Step 1: Preparation of (S)-5-(3-((tert-butyldimethylsilyl)oxy)pyrrolidin-1-yl)-1-ethyl-6-nitro-1H-indazole

The title compound was prepared according to the procedure described in step-5 of example 11 by using (S)-5-(3-((tert-butyldimethylsilyl)oxy)pyrrolidin-1-yl)-6-nitro-1H-indazole (product of step 04 of example 11) (400 mg, 1.1 mmol) under the same reaction conditions. Yield: 300 mg (69.9%). LCMS: 66.2%, m/z=391.4 (M+1).

### Step 2: Preparation of (S)-5-(3-((tert-butyldimethylsilyl)oxy)pyrrolidin-1-yl)-1-ethyl-1H-indazol-6-amine

The title compound was prepared according to the procedure described in step-6 of example 11 by using (S)-5-(3 -((tert-butyldimethylsilyl)oxy)pyrrolidin-1-yl)-1-ethyl-6-nitro-1H-indazole (300 mg, 0.77 mmol) under the same reaction conditions. Yield: 200 mg (72.2%). LCMS: 92.5%, m/z=361.7 (M+1)

### Step 3: Preparation of (S)—N—(1-ethyl-5-(3-hydroxypyrrolidin-1-yl)-1H-indazol-6-yl)-2-(2-methylpyridin-4-yl)oxazole-4-carboxamide hydrochloride

Using the same reaction conditions as described in step-7 of example 11, (S)-5-(3-((tert-butyldimethylsilyl)oxy)pyrrolidin-1-yl)-1-ethyl-1H-indazol-6-amine (200 mg, 0.6 mmol) was reacted with 2-(2-methylpyridin-4-yl)oxazole-4-carboxylic acid (Intermediate 4) (136 mg, 0.660 mmol). This was further treated with methanolic HCl to obtain the title compound. Yield: 100 mg (62.50%).

¹HNMR (CD3OD, 400 MHz): δ 8.99 (s, 1H), 8.91 (d, 1H), 8.64 (s, 1H), 8.55 (d, 1H), 8.4-8.3 (bs, 1H), 8.18 (s, 1H), 8.0-7.9 (s, 1H), 4.74 (s, 1H), 4.51 (q, 2H), 4.0-3.9 (m, 3H), 3.66 (d, 1H), 2.92 (s, 3H), 2.50-2.30 (m, 2H), 1.49 (t, 3H). LCMS: 92.3%, m/z=433.3 (M+1). HPLC: 97.97%.

### Example 33

(S)—N—(1-cyclopropyl-5-(3-hydroxypyrrolidin-1-yl)-1H-indazol-6-yl)-2-(2-methylpyridin-4-yl)oxazole-4-carboxamide hydrochloride

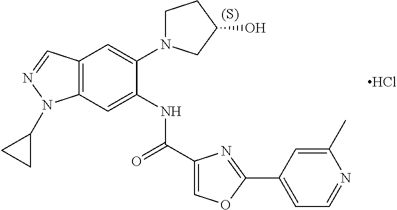

Step 1: Preparation of ((S)-5-(3-((tert-butyldimethylsilyl)oxy)pyrrolidin-1-yl)-1-cyclopropyl-6-nitro-1H-indazole

The solution of (S)-5-(3-((tert-butyldimethylsilyl)oxy)pyrrolidin-1-yl)-6-nitro-1H-indazole (product of step 04 of example 11) (600 mg, 1.6 mmol), Cyclopropyl boronic acid (280 mg, 3.3 mmol), copper acetate (300 mg, 0.16 mmol), 2,2′-bipyridine (260 mg, 1.6 mmol) in EDC (15 ml) was heated at 70° C. for 2h. The reaction mixture was filtered over Celite® and the filtrate was concentrated. This was purified by silica gel column chromatography and eluted with 10% ethyl acetate in hexane to obtain the title compound as the nonpolar isomer (500 mg, 77.6%). LCMS: 98.60%, m/z=403.0 (M+1).

### Step 2: Preparation of (S)-5-(3-((tert-butyldimethylsily)oxy)pyrrolidin-1-yl)-1-cyclopropyl-1H-indazol-6-amine

Using the same reaction conditions described in step-6 of example 11-(S)-5-(3-((tert-butyldimethylsilyl)oxy)pyrrolidin-1-yl)-1-cyclopropyl-6-nitro-1H-indazole (500 mg, 1.2 mmol) in THF (10 mL) was added ammonium chloride (800 mg, 15 mmol) in water (3 mL) and zinc dust (650 mg, 9.9 mmol) and stirred at RT for 30 min The catalyst was filtered through Celite®. The compound was extracted with DCM (2*100 mL) and the solvent was distilled out to obtain the crude product (300 mg, 67.2%). LCMS: 98.6%, m/z=374.3 (M+1)

### Step 3: Preparation of (S)—N—(1-cyclopropyl-5-(3-hydroxypyrrolidin-1-yl)-1H-indazol-6-yl)-2-(2-methylpyridin-4-yl)oxazole-4-carboxamide hydrochloride

Using the same reaction conditions as described in step-7 of example 11, (S)-5-(3-((tert-butyldimethylsilyl)oxy)pyrrolidin-1-yl)-1-cyclopropyl-1H-indazol-6-amine (300 mg, 0 8 mmol) was reacted with 2-(2-methylpyridin-4-yl)oxazole-4-carboxylic acid (Intermediate 4) (200 mg, 0.960 mmol).This was further treated with methanolic HCl to obtain the title compound (100 mg, 61.3%).

¹HNMR (CD₃OD, 400 MHz): δ 8.99 (s, 1H), 8.91 (d, 1H), 8.65 (s, 1H), 8.55 (d, 1H), 8.4-8.3 (bs, 1H), 8.12 (s, 1H), 8.0-7.9 (s, 1H), 4.74 (s, 1H), 3.90-3.85 (m, 2H), 3.73-3.69 (m, 2H), 2.93 (s, 3H), 2.50-2.30 (m, 2H), 1.28-1.18 (m, 4H). LCMS: 99.56%, m/z=445.2 (M+1). HPLC: 97.67%.

### Example 34

(S)—N—(5-(3-hydroxypyrrolidin-1-yl)-1-methyl-1H-indazol-6-yl)-2-(1,2,3,6-tetrahydropyridin-4-yl)oxazole-4-carboxamide hydrochloride

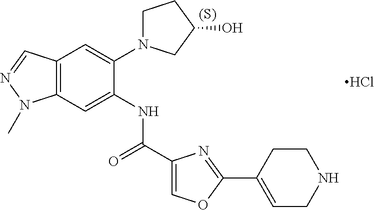

Using the same reaction conditions as described in step-7 of example 11, (S)-5-(3-((tert-butyldimethylsilyl)oxy)pyrrolidin-1-yl)-1-methyl-1H-indazol-6-amine (product of step-6 of example 11) (134 mg, 0.387 mmol) was reacted with 2-(1-(tert-butoxycarbonyl)-1,2,3,6-tetrahydropyridin-4-yl)oxazole-4-carboxylic acid (Intermediate 10) (100 mg, 0. 387 mmol). This was then treated with methanolic HCl to obtain the title compound (22 mg, 36%).

¹HNMR (DMSO-d₆, 400 MHz): δ 10.25 (s, 1H), 9.37 (s, 1H), 8.98 (s, 1H), 8.44 (s, 1H), 7.39 (s, 1H), 7.68 (s, 1H), 6.81 (s, 1H), 4.50-4.45 (m, 1H), 3.85-3.81 (m, 3H), 3.43-3.16 (m, 4H), 3.10-2.70 (m, 6H), 2.33-2.50 (m, 2H), 1.90-1.85 (m, 1H). LCMS: 98.6%, m/z=409.3 (M+1). HPLC: 91.38%.

### Example 35

(S)—N—(5-(3-hydroxypyrrolidin-1-yl)-1-methyl-1H-indazol-6-yl)-2-(2-methylpyrimidin-4-yl)oxazole-4-carboxamide hydrochloride

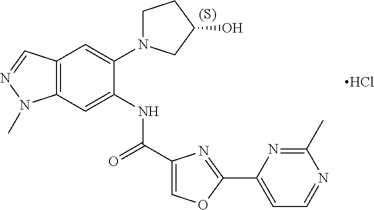

Using the same reaction conditions as described in step 7 of example 11, 2-(2-methylpyrimidin-4-yl)oxazole-4-carboxylic acid (intermediate 13) (85 mg, 0.414 mmol) was reacted with (S)-5-(3-((tert-butyldimethylsilyl)oxy)pyrrolidin-1-yl)-1-methyl-1H-indazol-6-amine (150 mg, 0.433 mmol). This was further treated with methanolic HCl to get the title compound (115 mg, 81.5%).

¹HNMR (DMSO-d₆, 400 MHz): δ 10.39 (s, 1H), 9.10 (s, 1H), 8.96 (d, 1H), 8.46 (s, 1H), 8.04 (d, 1H), 7.90 (s, 1H), 7.64 (s, 1H), 5.10 (bs, 1H), 4.55-4.50 (m, 1H), 3.98 (s, 3H), 3.32-3.10 (m, 2H), 3.01-2.92 (m, 2H), 2.92 (s, 3H), 2.34-2.30 (m, 1H), 1.95-1.90 (m, 1H). LCMS: 90.12%, m/z=420.3 (M+1). HPLC: 98.74%.

### Example 36

### (S)—N—(5-(3-hydroxypyrrolidin-1-yl)-1-methyl-1H-indazol-6-yl)-4-methyl-2-(2-methylpyrimidin-4-yl)oxazole-5-carboxamide hydrochloride

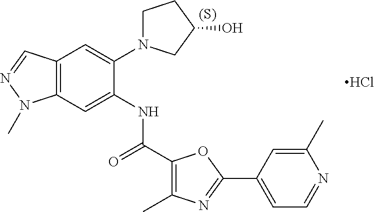

Using the same reaction conditions as described in step 7 of example 11, 4-methyl-2-(2-methylpyridin-4-yl)oxazole-5-carboxylic acid (step-4 of Intermediate 5) (113 mg, 0.52 mmol) was reacted with (S)-5 -(3-((tert-butyldimethylsilyl)oxy)pyrrolidin-1-yl)-1 -methyl-1H-indazol-6-amine (150 mg, 0.433 mmol). This was further treated with methanolic HCl to obtain the title compound (120 mg, 50.84%).

¹HNMR (DMSO-d₆, 300 MHz): δ 10.07 (s, 1H), 8.65 (d, 1H), 8.37 (s, 1H), 7.97 (s, 1H), 7.90-7.85 (m, 2H), 7.60 (s, 1H), 5.12 (s, 1H), 4.50-4.45 (m, 1H), 3.98 (s, 3H), 3.21-3.16 (m, 3H), 3.02-3.92 (m, 3H), 2.58 (s, 3H), 2.55 (s, 3H). LCMS: 98.34%, m/z=432.9 (M+1). HPLC: 99.55%.

### Example 37

(S)—N—(5-(3-hydroxypyrrolidin-1-yl)-1-methyl-1H-indazol-6-yl)-2-(piperidin-4-yl)oxazole-4-carboxamide hydrochloride

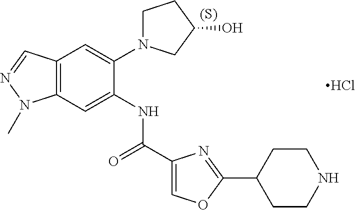

### Step-1: Preparation of tert-butyl (S)-4-(4-((5-(3-((tert-butyldimethylsilyl)oxy)pyrrolidin-1-yl)-1-methyl-1H-indazol-6-yl)carbamoyl)oxazol-2-yl)-3,6-dihydropyridine-1(2H)-carboxylate

Using the same reaction conditions as described in step-7 of example 11, 2-(1-(tert-butoxycarbonyl)-1,2,3,6-tetrahydropyridin-4-yl)oxazole-4-carboxylic acid (intermediate 10, step-2) (100 mg, 0.387 mmol) was reacted with (S)-5-(3-((tert-butyldimethylsilyl)oxy)pyrrolidin-1-yl)-1)-1-methyl-1H-indazol-6-amine (134 mg, 0.387 mmol), to obtain the title compound (200 mg, 82.9%). LCMS: 98.34%, m/z=623.1 (M+1).

### Step-2: Preparation of tert-butyl (S)-4-(4-45-(3-((tert-butyldimethylsilyl)oxy)pyrrolidin-1-yl)-1-methyl-1H-indazol-6-yl)carbamoyl)oxazol-2-yl)piperidine-1-carboxylate

The solution of tert-butyl (S)-4-(4-((5 -(3 -((tert-butyldimethylsilyl)oxy)pyrrolidin-1-yl)-1-methyl-1H-indazol-6-yl)carbamoyl)oxazol-2-yl)-3,6-dihydropyridine-1(2H)-carboxylate (intermediate 10, step-2) (75 mg, 0.12 mmol) in ethanol (5 mL) was hydrogenated with 10% Pd/c in presence of H2 balloon pressure for 12 h. After completion of reaction, the catalyst was filtered through Celite® and concentrated to obtain the crude product (60 mg). LCMS: 94.19%, m/z=625.5 (M+1).

### Step-3: Preparation of (S)—N—(5-(3-hydroxypyrrolidin-1-yl)-1-methyl-1H-indazol-6-yl)-2-(piperidin-4-yl)oxazole-4-carboxamide hydrochloride

A solution of tert-butyl (S)-4-(4-((5-(3-((tert-butyldimethylsilyl)oxy)pyrrolidin-1-yl)-1-methyl-1H-indazol-6-yl)carbamoyl)oxazol-2-yl)piperidine-1-carboxylate (60 mg, 0.096 mmol) in MeOH (1 mL) and 1,4-Dioxane. HCl (1 mL) was added and the reaction mixture was stirred for 1 h at room temperature. After completion of reaction, concentrated under reduced pressure and washed with diethyl ether to obtain the title compound (23 mg, 46.0%).

¹HNMR (DMSO-d₆, 400 MHz): δ 10.18 (s, 1H), 8.25 (s, 1H), 8.43 (s, 1H), 7.93 (s, 1H), 7.69 (s, 1H), 4.48 (s, 1H), 3.99 (s, 3H), 3.40-3.20 (m, 5H), 3.16 (s, 2H), 3.10-2.85 (m, 4H), 2.25-2.20 (3H), 2.10-1.80 (m, 3H). LCMS: 100%, m/z=410.8 (M+1). HPLC: 92.35%.

### Example 38

N—(5-(3-hydroxy-8-azabicyclo[3.2.1]octan-8-yl)-1-methyl-1H-indazol-6-yl)-2-(2-methylpyridin-4-yl)oxazole-4-carboxamide

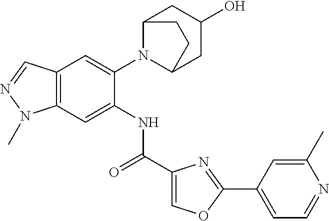 [SMILES: Cc1cc(-c2nc(C(=O)Nc3cc4c(cnn4C)cc3N3C4CCC3CC(O)C4)co2)ccn1]

The title compound was prepared according to the procedure described in product of step-1 to step 7 of example 11 by using the appropriate reactants and reaction conditions. Yield: 35 mg (28.0%).

¹HNMR (CDCl₃, 400 MHz): δ 10.48 (s, 1H), 8.68 (d, 2H), 8.43 (s, 1H), 7.84 (s, 1H), 7.74 (s, 1H), 7.66 (d, 1H), 7.30 (s, 1H), 4.42 (s, 1H), 4.06 (s, 3H), 3.66 (bs, 2H), 2.80-2.75 (m, 3H), 2.67 (s, 3H), 2.40-2.35 (m, 3H), 2.20-2.15 (m, 2H), 2.01 (d, 1H). LCMS: 98.77%, m/z=459.25 (M+1). HPLC: 98.76%.

### Example 39

(S)—N—(5-(3-hydroxypyrrolidin-1-yl)-1-methyl-1H-indazol-6-yl)-2-(2-methylpyridin-4-yl)oxazole-5-carboxamide

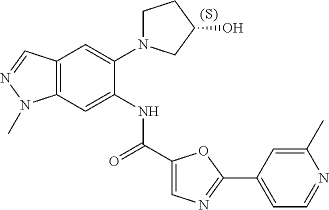

Using the same reaction conditions as described in step-7 of example 11, 2-(2-methylpyridin-4-yl)oxazole-5-carboxylic acid (intermediate 4, step-2) (106 mg, 0.52 mmol) was reacted with (S)-5 -(3-((tert-butyldimethylsilyl)oxy)pyrrolidin-1-yl)-1 -methyl-1H-indazol-6-amine (step-6 of example 11) (150 mg, 0.433 mmol) to obtain the title compound (23 mg, 72.0%)

¹HNMR (DMSO-d₆, 400 MHz): δ 10.20 (s, 1H), 8.68 (d, 1H), 8.22 (d, 2H), 7.98 (s, 1H), 7.93-7.89 (m, 2H), 7.54 (s, 1H), 5.12 (s, 1H), 4.45-4.40 (m, 1H), 4.00 (s, 3H), 3.22-3.19 (m, 2H), 3.04 (d, 2H), 2.61 (s, 3H), 2.33-2.25 (m, 1H), 1.95-1.90 (m, 1H). LCMS: 97.94%, m/z=419.0 (M+1). HPLC: 95.09%.

### Example 40

N-(5-(4-hydroxy-4-(hydroxymethyl)piperidin-1-yl)-1-methyl-1H-indazol-6-yl)-2-(2-methylpyridin-4-yl)oxazole-4-carboxamide

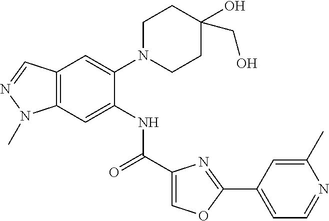 [SMILES: Cc1cc(-c2nc(C(=O)Nc3cc4c(cnn4C)cc3N3CCC(O)(CO)CC3)co2)ccn1]

The title compound was prepared according to the procedure described in step-1 to step 7 of example 11 by using the appropriate reactants and reaction conditions.

¹HNMR (DMSO-d₆, 400 MHz): δ 10.54 (s, 1H), 9.19 (s, 1H), 8.78 (d, 1H), 8.54 (s, 1H), 8.24 (d, 1H), 8.19 (s, 1H), 7.90 (s, 1H), 7.63 (s, 1H), 3.96 (s, 3H), 3.36-3.30 (m, 3H), 3.12-3.00 (m, 2H), 2.80-2.70 (m, 5H), 2.15-2.05 (m, 2H), 1.60-1.50 (m, 2H). LCMS: 99.00%, m/z=463.25 (M+1). HPLC: 95.03%.

### Example 41

(S)—N—(5-(3-hydroxypyrrolidin-1-yl)-1-methyl-1H-indazol-6-yl)-5-methyl-2-(2-methylpyridin-4-yl)oxazole-4-carboxamide hydrochloride

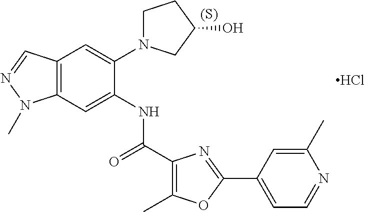

Using the same reaction conditions as described in step-7 of example 11, (S)-5-(3-((tert-butyldimethylsilyl)oxy)pyrrolidin-1-yl)-1-methyl-1H-indazol-6-amine (product of step-6 of example 11) (150 mg, 0.346 mmol) was reacted with 5-methyl-2-(2-methylpyridin-4-yl)oxazole-4-carboxylic acid (Intermediate 5) (89 mg, 0. 416 mmol). This was further treated with methanolic HCl to obtain the title compound (70 mg, 70.0%).

¹HNMR (CDCl₃, 300 MHz): δ 10.53 (s, 1H), 8.67 (d, 1H), 8.61 (s, 1H), 7.87 (s, 1H), 7.83 (s, 1H), 7.75 (d, 1H), 7.55 (s, 1H), 4.65-4.55 (m, 1H), 4.08 (s, 3H), 3.43-3.39 (m, 1H), 3.25 (d, 1H), 3.14 (dd, 1H), 2.97 (q, 1H), 2.86 (s, 3H), 2.67 (s, 3H), 2.65-2.60 (m, 1H), 2.60-2.50 (m, 1H), 2.20-2.10 (m, 1H). LCMS: 99.58%, m/z=432.9 (M+1). HPLC: 96.85%.

### Example 42

(S)-2-(2-ethylpyridin-4-yl)—N—(5-(3-hydroxypyrrolidin-1-yl)-1-methyl-1H-indazol-6-yl)oxazole-4-carboxamide hydrochloride

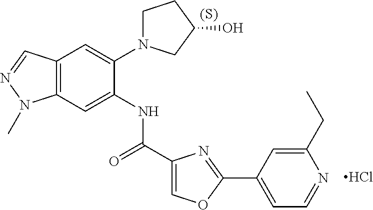

Using the same reaction conditions as described in step-7 of example 11, (S)-5-(3-((tert-butyldimethylsilyl)oxy)pyrrolidin-1-yl)-1-methyl-1H-indazol-6-amine(product of step-6 of example 11) (150 mg, 0.346 mmol) was reacted with 2-(2-ethylpyridin-4-yl)oxazole-4-carboxylic acid (Intermediate 12) (94 mg, 0.414 mmol). This was then treated with methanolic HCl to obtain the title compound (7 mg, 12%).

¹HNMR (CD3OD, 400 MHz): δ 8.62 (s, 1H), 8.53 (d, 1H), 8.44 (s, 1H), 7.92 (brs, 1H), 7.83-7.79 (m, 2H), 7.58 (s, 1H), 4.67-4.63 (m, 1H), 3.94 (s, 3H), 3.40-3.36 (m, 3H), 3.15-2.90 (m, 4H), 2.50-2.40 (m, 1H), 2.10-2.00 (m, 1H), 1.38-1.27 (m, 4H). LCMS: 91.71%, m/z=433.1 (M+1). HPLC: 95.03%.

### Example 43

2-(2-aminopyridin-4-yl)—N—(5-(4-(hydroxymethyl)piperidin-1-yl)-1,3-dimethyl-1H-indazol-6-yl)oxazole-4-carboxamide hydrochloride

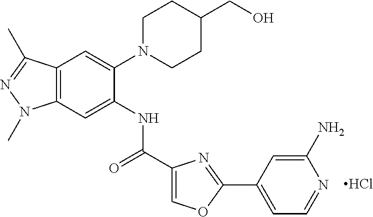 [SMILES: Cc1nn(C)c2cc(NC(=O)c3coc(-c4ccnc(N)c4)n3)c(N3CCC(CO)CC3)cc12 ; Cl]

### Step 1: Preparation of 1-(5-fluoro-1H-indazol-1-yl)ethan-1-one

Using the same reaction conditions described in step 2 of example 11, acetic anhydride (12.24 g, 120 mmol) was added slowly to a mixture of 4-fluoro-2-methylaniline (5.0 g, 40 mmol) and potassium acetate (5 g, 52 mmol) in chloroform (50 ml) and stirred at 60° C. for one hour. After lhr, reaction mixture was cooled again to room temperature and isoamylnitrite (9.28 g, 80 mmol) was added and further heated to 75° C. for overnight. The reaction mixture was diluted with DCM washed with water and brine solution dried over Na₂SO₄ and evaporated. The crude compound was purified by column chromatography eluted with 50% ethyl acetate in hexane to obtain the title compound (1.8 g, 25.3%). LCMS: m/z=178.0.

Step 2: Preparation of 5-fluoro-1H-indazole

A mixture of 1-(5-fluoro-1H-indazol-1-yl) ethan-1-one (2.1 g, 11 8 mmol) in methanol (20 ml) and concentrated hydrochloric acid (10 ml) was heated to 50° C. for 2 hrs. After the completion of reaction, reaction mixture was evaporated to dryness under reduced pressure. The residue was basified with saturated sodium bicarbonate solution and extracted to ethyl acetate, washed with water and concentrated to obtain the title compound (1.6 g, 100%). LCMS: 95.9%; m/z=137.2.

### Step 3: Preparation of 3-bromo-5-fluoro-1H-indazole

N-Bromosuccinimide (2.09 g, 11.76 mmol) was added in several portions to a solution of 5-fluoro-1H-indazole (1.6 g, 11.76 mmol) at 0° C. and thereafter stirred at room temperature for 2h. After the completion of reaction, reaction mixture was diluted with DCM, washed with water and concentrated to obtain the title compound (1.5 g, 59.3%). LCMS: 95.6%; m/z=214.9

Step 4: Preparation of 3-bromo-6-nitro-1H-indazol-5-ol

3-bromo-5-fluoro-1H-indazole (1.2 g, 5.63 mmol) was added in several portions to a cooled and stirred nitrating mixture (5ml sulphuric acid+5ml nitric acid) at −10° C. and thereafter stirred at room temperature for 3h. After the completion of reaction, reaction mixture was quenched over crushed ice and the yellow solid was filtered and dried to obtain the title compound (800 mg, 55.05%). LCMS: 82.7%; m/z=259.95

### Step 5: Preparation of 3-bromo-6-nitro-1H-indazol-5-ylmethanesulfonate

Methanesulfonyl chloride (424 mg, 3.72 mmol) was added to a mixture of 3-bromo-6-nitro-1H-indazol-5-ol (800 mg, 3.1 mmol) in DCM and triethylamine (1.3 ml, 9.3 mmol) at 0° C. and then stirred at room temperature for 2 hrs. After the completion of reaction, reaction mixture was diluted with DCM, washed with water and concentrated to obtain the title compound (1.0 g, 80.1%).

### Step 6: Preparation of (1-(3-bromo-6-nitro-1H-indazol-5-yl)piperidin-4-yl)methanol

(Piperidin-4-yl)methanol (513 mg, 4.464 mmol) was added to a mixture of 3-bromo-6-nitro-1H-indazol-5-yl methanesulfonate (1 g, 2.976 mmol) and potassium carbonate (1.23 g, 8.928 mmol) in DMF (10 ml) at 0° C. and thereafter stirred at room temperature for 16h. After the completion of reaction, reaction mixture was poured over ice water and extracted with ethyl acetate and concentrated to get the title compound (1.2 g crude, 100%). LCMS: 84.7%, m/z=355.0 (M+1)

### Step 7: Preparation of 3-bromo-5-(4-(((tert-butyldimethylsilyl)oxy)methyl)piperidin-1-yl)-6-nitro-1H-indazole

TBDMS chloride (606 mg, 4.04 mmol) was added to a cooled mixture of (1-(3-bromo-6-nitro-1H-indazol-5-yl)piperidin-4-yl)methanol (1.2 g, 3.3 mmol) and imidazole (673 mg, 9.9 mmol) in DMF at 0° C. and thereafter stirred at room temperature for 6h. After the reaction mixture was poured over ice water and extracted to ethyl acetate and concentrated. The crude compound was purified by silica gel column chromatography and eluted with 0-20% ethyl acetate in hexanes to obtain the title compound (1.5 g, 96%). LCMS: 90.38%, m/z=471.5 (M+1)

### Step 8: Preparation of 3-bromo-5-(4-(((tert-butyldimethylsilyl)oxy)methyl)piperidin-1-yl)-1-methyl-6-nitro-1H-indazole

To a solution of sodium hydride (84.8 mg, 2.12 mmol) in DMF (5 mL), 3-bromo-5-(4-(((tert-butyldimethylsilyl)oxy)methyl)piperidin-1-yl)-6-nitro-1H-indazole (800 mg, 1.72 mmol) was added at 0° C. After 15 min to that solution at 0° C. methyl iodide (365 mg, 2.59 mmol) was added. The reaction mixture was allowed to room temperature for 2h. The reaction mixture was diluted with EtOAc, washed with brine and dried over anhydrous Na₂SO_{4.} This was purified by silica gel column chromatography and eluted with 20% ethyl acetate in hexane to obtain the title compound (800 mg, 97%). LCMS: 98.7%, m/z=485.0 (M+1)

### Step 9: Preparation of 5-(4-(((tert-butyldimethylsilyl)oxy)methyl)piperidin-1-yl)-1,3-dimethyl-6-nitro-1H-indazole

A solution of 3-bromo-5-(4-(((tert-butyldimethylsilyl)oxy)methyl)piperidin-1-yl)-1-methyl-6-nitro-1H-indazole (800 mg, 1.652 mmol), methylboronic acid (146 mg, 2.48 mmol), tricyclohexylphosphine (92 mg, 0.33 mmol), palladium acetate (37 mg, 0.165 mmol), tripotassium phosphate (1.05 g, 4.956 mmol) in toluene (10 ml) and water (2 ml) was heated in a sealed tube at 110° C. for overnight. After the reaction, reaction mixture was diluted with ethyl acetate and filtered over Celite® and the filtrate was concentrated. The crude compound was purified over silica gel column chromatography eluted with 30% ethyl acetate in hexanes to obtain the title compound (600 mg, 86%). LCMS: 95.9%, m/z=419.4 (M+1)

### Step 10: Preparation of 5-(4-(((tert-butyldimethylsily)oxy)methyl)piperidin-1-yl)-1,3-dimethyl-1H-indazol-6-amine

To a solution of 5-(4-(((tert-butyldimethylsilyl)oxy)methyl)piperidin-1-yl)-1,3-dimethyl-6-nitro-1H-indazole (600 mg, 1.43 mmol) in THF (12 mL) ammonium chloride (757 mg, 14.3 mmol) in water (3 mL) and zinc dust (466 mg, 7.17 mmol) were added and stirred at RT for 30 min. The catalyst was filtered through Celite®. The compound was extracted with DCM (2*100 mL) and distilled out the solvent to obtain the crude product (360 mg, 64.9%). LCMS: 96.4%, m/z=390.2 (M+1)

### Step 11: Preparation of −(2-aminopyridin-4-yl)—N—(5-(4-(hydroxymethyl)piperidin-1-yl)-1,3-dimethyl-1H-indazol-6-yl)oxazole-4-carboxamide hydrochloride

Using the same reaction conditions described in step 7 of example 11, 5-(4-(((tert-butyldimethylsilyl)oxy)methyl)piperidin-1-yl)-1,3-dimethyl-1H-indazol-6-amine (150 mg, 0.386 mmol) in DMF (3 mL) was reacted with 2-(2-aminopyridin-4-yl)oxazole-4-carboxylic acid (93.7 mg, 0.464 mmol. This was then treated with methanolic HCl to obtain the title compound (45 mg, 46.8%)

¹HNMR (CDCl₃, 400 MHz): δ 9.32 (s, 1H), 8.41 (s,1H), 8.23 (d, 1H), 7.30-7.25 (m, 3H), 7.19 (d, 1H), 4.81 (s, 2H), 3.96 (s, 3H), 3.56 (d, 2H), 3.01 (d, 2H), 2.78 (t, 2H), 2.59 (s, 3H), 1.70-1.60 (m, 4H). LCMS: 100%, m/z=462.1 (M+1). HPLC: 98.67%.

### Example 44

(S)—N—(5-(3-hydroxypyrrolidin-1-yl)-1-(piperidin-4-ylmethyl)-1H-indazol-6-yl)-2-(2-methylpyridin-4-yl)oxazole-4-carboxamide hydrochloride

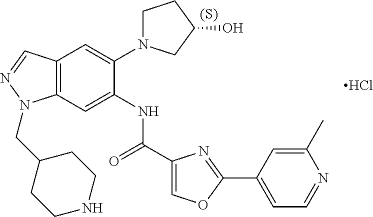

### Step-1: Preparation of tert-butyl (S)-4-((5-(3-((tert-butyldimethylsilyl)oxy)pyrrolidin-1-yl)-6-nitro-1H-indazol-1-yl)methyl)piperidine-1-carboxylate

To a solution of (S)-5-(3-((tert-butyldimethylsilyl)oxy)pyrrolidin-1-yl)-6-nitro-1H-indazole (step-4 of example 11) (800 mg, 2.209 mmol) in DMF (5 mL) K₂CO₃ (618 mg, 4.419 mmol) was added at 0° C. After 15 min, at 0° C., tert-butyl 4-(bromomethyl) piperidine-1-carboxylate (730 mg, 2.651 mmol) was added. The reaction mixture was heated to 100° C. for 12h. The reaction mixture was quenched with water and diluted with EtOAc, washed with brine and dried over anhydrous Na₂SO₄. This was purified by silica gel column chromatography and elution with 20% ethyl acetate in hexane gave isomer A tert-butyl (S)-4-((5-(3-((tert-butyldimethylsilyl)oxy)pyrrolidin-1-yl)-6-nitro-1H-indazol-1-yl)methyl)piperidine-1-carboxylate (630 mg, 51.2%). LCMS: 97.8%, m/z=560.2 (M+1).

### Step-2: tert-butyl (S)-4-((6-amino-5-(3-((tert-butyldimethylsilyl)oxy)pyrrolidin-1-yl)-1H-indazol-1-yl)methyl)piperidine-1-carboxylate

To a solution of tert-butyl (S)-4-((5-(3-((tert-butyldimethylsilyl)oxy)pyrrolidin-1-yl)-6-nitro-1H-indazol-1-yl)methyl)piperidine-1-carboxylate (product of step-1 isomer-B of example -44) (630 mg, 1.125 mmol) in THF (10 mL) ammonium chloride (0962 mg, 180 mmol) in water (2 mL) and zinc dust (588 mg, 9.0 mmol) were added and stirred at RT for 30 min. The catalyst was filtered through Celite®. The compound was extracted with ethyl acetate and the solvent was distilled out to obtain the title compound (450 mg, 96.2%). LCMS: 97.1%, m/z=530.3 (M+1).

### Step-3: (S)—N—(5-(3-hydroxypyrrolidin-1-yl)-1-(piperidin-4-ylmethyl)-1H-indazol-6-yl)-2-(2-methylpyridin-4-yl)oxazole-4-carboxamide hydrochloride

Using the same reaction conditions described in step 7 of example 11, tert-butyl (S)-4-((6-amino-5-(3-((tert-butyldimethylsilyl)oxy)pyrrolidin-1-yl)-1H-indazol-1-yl)methyl)piperidine-1-carboxylate (450 mg, 0.0.85 mmol) was reacted with 2-(2-methylpyridin-4-yl)oxazole-4-carboxylic acid (260 mg, 0. 1.27mmol. It was then treated with methanolic HCl to obtain the title compound (200 mg, 67.56%).

¹HNMR (DMSO-d₆, 400 MHz): δ 10.42 (s, 1H), 9.05 (s, 1H), 8.68 (d, 1H), 8.54 (s, 1H), 7.98 (s, 1H), 7.92 (s, 1H), 7.80 (d, 1H), 7.68 (s, 1H), 5.14 (d, 1H), 4.60-4.50 (m, 1H), 4.27 (d, 2H), 3.36-3.21 (m, 6H), 3.02-2.97 (m, 2H), 2.83 (t, 2H), 2.59 (s, 3H), 2.36-2.31 (m, 1H), 2.25-2.15 (m, 1H), 1.70-1.60 (m, 2H), 1.50-1.40 (m, 2H). LCMS: 98.9%, m/z=502.5 (M+1). HPLC: 98.56%.

### Example 45

N—(5-(4-(hydroxymethyl)piperidin-1-yl)-1,3-dimethyl-1H-indazol-6-yl)-2-(2-methylpyridin-4-yl)oxazole-4-carboxamide hydrochloride

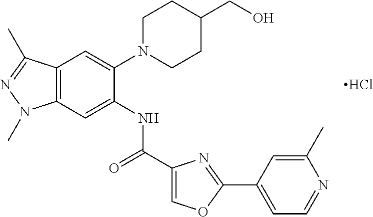 [SMILES: Cc1cc(-c2nc(C(=O)Nc3cc4c(cc3N3CCC(CO)CC3)c(C)nn4C)co2)ccn1 ; Cl]

Using the same reaction conditions described in step 7 of example 11, 5-(4-(((tert-butyldimethylsilyl)oxy)methyl)piperidin-1-yl)-1,3-dimethyl-1H-indazol-6-amine (step 10, example 43) (150 mg, 0.386 mmol) in DMF (3 mL) was reacted with 2-(2-methylpyridin-4-yl)oxazole-4-carboxylic acid (Intermediate 4) (93.7 mg, 0.464 mmol) It was then treated with methanolic HCl to obtain the title compound (90 mg, 78.2%).

¹HNMR (CDCl₃, 400 MHz): δ 9.26 (s, 1H), 8.70 (d,1H), 8.46 (s, 1H), 7.86 (s, 1H), 7.79 (d, 1H), 7.30 (s, 1H), 7.22 (d, 1H), 3.98 (s, 3H), 3.55-3.50 (m, 2H), 3.02 (d, 2H), 2.78 (t, 2H), 2.70 (s, 3H), 2.59 (s, 3H), 1.80-1.70 (m, 2H) 1.50-1.35 (m, 3H). LCMS: 100%, m/z=462.0 (M+1). HPLC: 98.23%.

### Example 46

(S)-2-(2-cyclopropylpyridin-4-yl)—N—(5-(3-hydroxypyrrolidin-1-yl)-1-methyl-1H-indazol-6-yl)oxazole-4-carboxamide hydrochloride

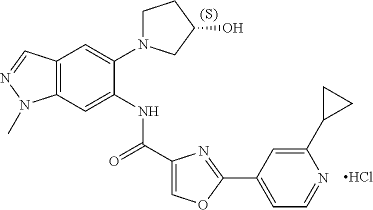

Using the same reaction conditions described in step 7 of example 11, (S)-5-(3-((tert-butyldimethylsilyl)oxy)pyrrolidin-1-yl)-1-methyl-1H-indazol-6-amine (product of step-6 of example 11) (100 mg, 0.29 mmol) in DMF (4 mL) 2-(2-cyclopropylpyridin-4-yl)oxazole-4-carboxylic acid (Intermediate 9) (100 mg, 0.43 mmol), HATU (220 mg, 0.57 mmol), DIPEA (149 mg, 1.15 mmol) were added The reaction mixture was stirred for 24h at room temperature and quenched with ice water and filtered to obtain crude product. This was then treated with methanolic HCl to obtain the desired compound (25 mg).

¹HNMR (CD3OD, 300 MHz): δ 8.71 (s, 1H), 8.57-8.54 (m, 2H), 7.94-7.90 (m, 2H), 7.82 (dd, 1H), 7.68 (s, 1H), 4.70-4.60 (m, 1H), 4.04 (s, 3H), 3.12-3.03 (m, 5H), 2.50-2.40 (m, 1H), 2.30-2.00 (m, 1H), 1.15-1.05 (m, 4H). LCMS: 96.42%, m/z=444.9 (M+1). HPLC: 97.93%.

### Example 47

N—(5-(4-hydroxypiperidin-1-yl)-2-methyl-2H-indazol-6-yl)pyrazolo[1,5-a]pyrimidine-3-carboxamide hydrochloride

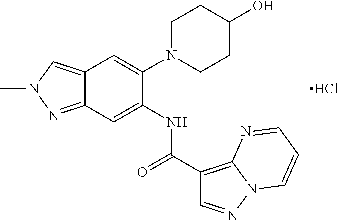 [SMILES: Cn1cc2cc(N3CCC(O)CC3)c(NC(=O)c3cnn4cccnc34)cc2n1 ; Cl]

### Step-1: Preparation 5-(4-((tert-butyldimethylsilyl)oxy)piperidin-1-yl)-2-methyl-6-nitro-2H-indazole

Using the same reaction conditions as described in step-5 of example 11, sodium hydride (152 mg, 3.79 mmol) in THF (20 mL), 5-(4-((tert-butyldimethylsilyl)oxy)piperidin-1-yl)-6-nitro-1H-indazole (product of step-3 of example 17) (680 mg, 1.80 mmol) were added at 0° C. After 15 min, at 0° C., methyl iodide (1.02 g, 7.21 mmol) was added. The reaction mixture was allowed to room temperature for 2h. The reaction mixture was diluted with EtOAc, washed with brine and dried over anhydrous Na₂SO_{4.} This was purified by silica gel column chromatography and elution with 15% ethyl acetate in hexane to obtain the title compound (395 mg, 56%). LCMS: 40.0%, m/z=391.2 (M+1).

Step-2: 5-(4-((tert-butyldimethylsiyl)oxy)piperidin-1-yl)-2-methyl-2H-indazol-6-amine

Using the same reaction conditions as described in step-6 of example 11, 5-(4-((tert-butyldimethylsilyl)oxy)piperidin-1-yl)-2-methyl-6-nitro-2H-indazole (product of step-1 example −47) (400 mg, 1.024 mmol) in THF (20 mL), ammonium chloride (490 mg, 8.19 mmol) in water (10 mL) and zinc dust (532 mg, 8.19 mmol) were added and stirred at RT for 30 min The catalyst was filtered through Celite®. The compound was extracted with ethyl acetate and the solvent was distilled out to obtain the product (232 mg, 65.0%). LCMS: 97.7%, m/z=361.1 (M+1).

### Step-3: —(5-(4-hydroxypiperidin-1-yl)-2-methyl-2H-indazol-6-yl)pyrazolo[1,5-a]pyrimidine-3-carboxamide hydrochloride

Using the same reaction conditions as described in step-6 of example 11, 5-(4-((tert-butyldimethylsilyl)oxy)piperidin-1-yl)-2-methyl-2H-indazol-6-amine (110 mg, 0.306 mmol) was reacted with pyrazolo[1,5-a]pyrimidine-3-carboxylic acid (50 mg, 0.306 mmol) This was then treated with methanolic HCl to obtain the title compound (39 mg, 34%).

¹HNMR (CD3OD, 300 MHz): δ 9.13 (dd, 1H), 9.00-8.96 (bs, 1H), 8.78 (s, 1H), 8.70 (s, 1H), 8.05 (s, 1H), 7.55 (s, 1H), 7.30-7.26 (m, 1H), 4.15 (s, 3H), 3.15-3.05 (m, 3H), 2.90-2.80 (m, 2H), 2.10-2.00 (m, 4H). LCMS: 100%, m/z=392.2 (M+1). HPLC: 98.65%.

### Example 48

(S)—N—(5-(3-hydroxypyrrolidin-1-yl)-1-methyl-1H-indazol-6-yl)pyrazolo[1,5-a]pyrimidine-3-carboxamide hydrochloride

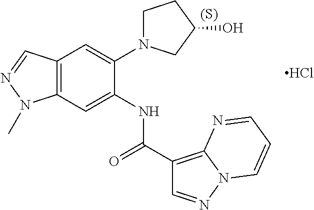

Using the same reaction conditions as described in step-6 of example 11, (S)-5-(3-((tert-butyldimethylsilyl)oxy)pyrrolidin-1-yl)-1-methyl-1H-indazol-6-amine (product of step-6 of example 11) (70 mg, 0.202 mmol) was reacted with pyrazolo[1,5-a]pyrimidine-3-carboxylic acid (39.5 mg, 0.242 mmol). This was then treated with methanolic HCl to obtain the desired compound (15 mg).

¹HNMR (DMSO-d₆, 400 MHz): δ 10.89 (s, 1H), 9.38 (d, 1H), 8.90 (d, 1H), 8.73 (s,1H), 8.67 (s, 1H), 7.90 (s, 1H), 7.65 (s, 1H), 7.37-7.34 (m, 1H) 4.55-4.50 (m, 1H), 3.98 (s, 3H), 3.47 (t, 1H), 3.22 (q, 1H), 3.10-3.00 (m, 2H), 2.84 (dd, 1H), 2.35-2.25 (m, 2H), 1.95-1.85 (m, 1H). LCMS: 98.9%, m/z=378.0 (M+1). HPLC: 96.63%.

### IRAK-4 Biochemical Assay

Compounds were tested for their potential to inhibit IRAK-4 enzyme in a TR-FRET assay using recombinant IRAK-4 kinase from Millipore, USA. The assay buffer was 50 mM Tris-HCl pH 7.5, 20 mM MgCl₂, 1 mM EGTA, 2 mM DTT, 3 mM MnCl₂ and 0.01% Tween 20.5 ng of IRAK-4 kinase was used for the assay. After pre-incubation of enzyme with test compound for 30 minutes at room temperature, a substrate mixture containing 100 nM Biotin Histone H3 (Millipore, USA) and 20 μM ATP (Sigma, USA) was added and the reaction was incubated for 30 min. Post incubation, the reaction was stopped by the addition of stop mix containing 40 mM EDTA, 1 nM of Europium-Anti-Phospho-Histone H3 (Ser10) antibody (Perkin Elmer, USA) and 20 nM SureLight Allophycocyanin-Streptavidin (Perkin Elmer, USA). The fluorescence emission at 615 nm and 665 nm were measured at an excitation of 340 nm and the percent inhibition was estimated from the ratio of the fluorescence intensities [(F665/F615)×10000]. The compounds were initially screened at 1 μM and 10 μM concentrations and potent compounds (>50% inhibition at 1 μM) were taken for dose response studies. The IC₅₀ values were estimated by fitting the dose-response data to sigmoidal dose response (variable slope), curve fitting program using Graphpad Prism software Version 6.01.

The compounds of the present invention were screened in the above mentioned assay and the results (percent inhibition and IC₅₀) are summarized in the Table 1. The IRAK-4 enzyme inhibitory rates at 0.1 μM and @1 μM are reported below. The IC50 values of the compounds of examples are set forth below wherein “A” refers to an IC50 value of less than 100 nM, “B” refers to IC₅₀ value ranges from 100.01 nM to 250 nM and “C” refers to an IC₅₀ value of greater than 250 nM.

TABLE 1
Percent inhibition and IC₅₀ values for
IRAK4 activity for selected compounds

| Example No | % inhibition @0.1 μM | % inhibition @1 μM | IC₅₀ (nM) |
|---|---|---|---|
| 1 | — | 96 | A |
| 2 | — | 98 | A |
| 3 | 90 | 73 | C |
| 4 | — | 84 | B |
| 5 | 95 | 82 | C |
| 6 | 96 | 90 | B |
| 7 | 93 | 83 | C |
| 8 | 90 | 56 | C |
| 9 | 83 | 78 | C |
| 10 | 95 | 91 | A |
| 11 | 95 | 98 | A |
| 12 | 94 | — | A |
| 13 | 79 | — | A |
| 14 | 94 | 96 | A |
| 15 | 83 | 99 | A |
| 16 | 94 | 99 | A |
| 17 | 93 | 95 | A |
| 18 | 94 | 96 | A |
| 19 | 94 | 97 | A |
| 20 | 96 | 97 | A |
| 21 | 41 | 84 | B |
| 22 | 89 | 96 | A |
| 23 | 81 | 95 | A |
| 24 | 33 | 80 | B |
| 25 | 24 | 71 | C |
| 26 | 96 | 92 | A |
| 27 | 88 | 97 | C |
| 28 | 96 | 98 | A |
| 29 | 14 | 95 | A |
| 30 | 0 | 20 | — |
| 31 | 0 | 37 | — |
| 32 | 62 | 95 | A |
| 33 | 25 | 87 | B |
| 34 | 0 | 41 | — |
| 35 | 45 | 93 | B |
| 36 | 2 | 1 | — |
| 37 | 0 | 0 | — |
| 38 | 5 | 77 | C |
| 39 | 49 | 92 | B |
| 40 | 95 | 98 | A |
| 41 | 0 | 17 | — |
| 42 | 83 | 97 | A |
| 43 | 0 | 15 | — |
| 44 | 0 | 54 | — |
| 45 | 2 | 12 | — |
| 46 | 93 | 97 | A |
| 47 | 34 | 89 | B |
| 48 | 25 | 80 | C |

1. A compound of formula (I): 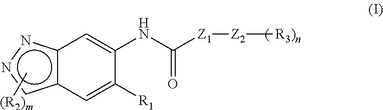
or a pharmaceutically acceptable salt or a stereoisomer thereof;
wherein,
Z₁ is optionally substituted cycloalkyl, optionally substituted aryl, optionally substituted heterocyclyl or is absent;
Z₂ is optionally substituted cycloalkyl, aryl or heterocyclyl;
R₁ is hydrogen, optionally substituted alkyl, amino, halogen, cyano, optionally substituted cycloalkyl, optionally substituted aryl, optionally substituted heterocyclyl, optionally substituted arylalkyl or optionally substituted heterocyclylalkyl;
R₂ at each occurrence is hydrogen, halogen, amino, optionally substituted alkyl, optionally substituted cycloalkyl, optionally substituted aryl, optionally substituted heterocyclyl, optionally substituted arylalkyl or optionally substituted heterocyclylalkyl;
R₃ at each occurrence is hydroxy, halogen, optionally substituted alkyl, optionally substituted alkoxy, optionally substituted cycloalkyl or —NRₐR_{b};
Rₐ and R_{b}, independently for each occurrence, are hydrogen, optionally substituted alkyl, optionally substituted acyl, optionally substituted cycloalkyl, optionally substituted aryl, optionally substituted heterocyclyl, optionally substituted arylalkyl or optionally substituted heterocyclylalkyl;
m, at each occurrence, is 0, 1 or 2; and
n, at each occurrence, is 0, 1, or 2.

2-26. (canceled)

27. The compound of claim 1, wherein
Z₁ is optionally substituted cycloalkyl, optionally substituted aryl, or optionally substituted heterocyclyl;
Z₂ is optionally substituted cycloalkyl, aryl or heterocyclyl;
R₁ is hydrogen, alkyl, amino, halogen, cyano, optionally substituted cycloalkyl, optionally substituted aryl, optionally substituted heterocyclyl, arylalkyl or heterocyclylalkyl;
R₂ is amino, alkyl, optionally substituted cycloalkyl, optionally substituted aryl, optionally substituted heterocyclyl, arylalkyl or heterocyclylalkyl;
R₃ is hydroxy, alkyl, alkoxy, or —NRaRb;
Rₐ and R_{b}, independently for each occurrence, are hydrogen, alkyl, optionally substituted cycloalkyl, optionally substituted aryl, optionally substituted heterocyclyl, arylalkyl or heterocyclylalkyl;
m is 1; and
n is 1.

28. The compound of claim 1, wherein
Z₁ is heterocyclyl;
Z₂ is heterocyclyl;
R₁ is optionally substituted heterocyclyl;
R₂ is alkyl;
R₃ is hydroxy, alkyl, or amino;
m is 1; and
nis 1.

29. The compound of claim 1, wherein Z₁ is an optionally substituted heterocyclyl.

30. The compound of claim 1, wherein Z₁ is a heterocyclyl selected from tetrazolyl, thienyl, triazolyl, pyrrolyl, pyridyl, pyranyl, pyrazinyl, pyridazinyl, pyrimidyl, imidazolyl, oxadiazolyl, thiadiazolyl, thiazolyl, isothiazolyl, oxazolyl, furanyl, pyrazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and pyrazolopyrimidyl.

31. The compound of claim 1, represented by formula (IA) 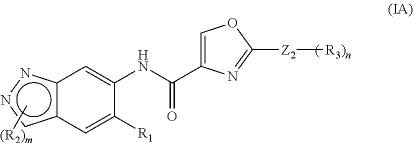
or a pharmaceutically acceptable salt thereof;
wherein Z₂, R₁, R₂, R₃, m, and n are as defined in claim 1.

32. The compound of claim 1, represented by formula (IB) 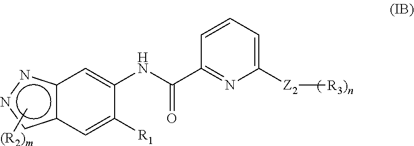
or a pharmaceutically acceptable salt thereof;
wherein, Z₂, R₁, R₂, R₃, m, and n are as defined in claim 1.

33. The compound of claim 1, wherein Z₂ is a heterocyclyl.

34. The compound of claim 1, wherein Z₂ is a heterocyclyl selected from azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, tetrazolyl, thienyl, triazolyl, pyrrolyl, pyridinyl, tetrahydropyridinyl, pyranyl, pyrazinyl, pyridazinyl, pyrimidyl, piperazinyl, imidazolyl, oxadiazolyl, thiadiazolyl, thiazolyl, isothiazolyl, oxazolyl, furanyl, pyrazolyl, indolinyl, indolinylmethyl, 2-aza-bicyclo[2.2.2]octanyl, chromanyl, xanthenyl or pyrrolopyridyl.

35. The compound of claim 1, wherein Z₂ is pyrrolidinyl, piperidinyl, piperazinyl, pyridinyl, pyrimidyl, tetrahydropyridinyl or pyrrolopyridyl.

36. The compound of claim 1, wherein Z₂ is pyrrolidinyl or pyridinyl.

37. The compound of claim 1, wherein R₁ is optionally substituted heterocyclyl.

38. The compound of claim 1, wherein R₁ is heterocyclyl; optionally substituted with halogen, hydroxyl or hydroxyalkyl.

39. The compound of claim 1, wherein R₁ is optionally substituted azetidinyl, piperidinyl, morpholinyl, pyrrolidinyl or azabicyclooctanyl.

40. The compound of claim 1, wherein R₁ is piperidinyl.

41. The compound of claim 1, wherein R₂ is optionally substituted alkyl.

42. The compound of claim 1, wherein R₂ is alkyl, optionally substituted with heterocyclyl.

43. The compound of claim 1, wherein R₂ is hydrogen.

44. The compound of claim 1, wherein R₂ is cyclopropyl.

45. The compound of claim 1, wherein R₃ is halogen, alkyl, haloalkyl, —NRₐR_{b}, cycloalkyl, hydroxyl or hydroxyalkyl; and Rₐ and R_{b} are as defined in claim 1.

46. The compound of claim 1, wherein R₃ is methyl, hydroxyl, or amino.

47. The compound of claim 1, wherein R₃ is hydroxyl or amino.

48. A compound selected from:

| Com- | |
|---|---|
| pound | |
| No. | IUPAC Name |
| 1. | N-(1-methyl-5-(piperidin-1-yl)-1H-indazol-6-yl)-2-(2- |
| | methylpyridin-4-yl)oxazole-4-carboxamide hydrochloride; |
| 2. | N-(2-methyl-5-(piperidin-1-yl)-2H-indazol-6-yl)-2-(2- |
| | methylpyridin-4-yl)oxazole-4-carboxamide hydrochloride; |
| 3. | (S)-6-(3-hydroxypyrrolidin-1-yl)-N-(2-methyl-5-(piperidin-1- |
| | yl)-2H-indazol-6-yl)picolinamide; |
| 4. | (S)-2-(3-aminopyrrolidin-1-yl)-N-(1-methyl-5-(piperidin-1-yl)- |
| | 1H-indazol-6-yl)oxazole-4-carboxamide; |
| 5. | (S)-2-(3-aminopyrrolidin-1-yl)-N-(2-methyl-5-(piperidin-1-yl)- |
| | 2H-indazol-6-yl)oxazole-4-carboxamide; |
| 6. | (S)-2-(3-hydroxypyrrolidin-1-yl)-N-(2-methyl-5-(piperidin-1- |
| | yl)-2H-indazol-6-yl)oxazole-4-carboxamide; |
| 7. | (S)-6-(3-aminopyrrolidin-1-yl)-N-(2-methyl-5-(piperidin-1-yl)- |
| | 2H-indazol-6-yl)picolinamide |
| 8. | (S)-6-(3-aminopyrrolidin-1-yl)-N-(1-methyl-5-(piperidin-1-yl)- |
| | 1H-indazol-6-yl)picolinamide; |
| 9. | (S)-6-(3-hydroxypyrrolidin-1-yl)-N-(1-methyl-5-(piperidin- |
| | 1-yl)-1H-indazol-6-yl)picolinamide; |
| 10. | (S)-2-(3-hydroxypyrrolidin-1-yl)-N-(1-methyl-5-(piperidin- |
| | 1-yl)-1H-indazol-6-yl)oxazole-4-carboxamide; |
| 11. | (S)-N-(5-(3-hydroxypyrrolidin-1-yl)-1-methyl-1H-indazol-6-yl)- |
| | 2-(2-methylpyridin-4-yl)oxazole-4-carboxamide hydrochloride; |
| 12. | N-(5-(3-hydroxypiperidin-1-yl)-1-methyl-1H-indazol-6-yl)-2-(2- |
| | methylpyridin-4-yl)oxazole-4-carboxamide hydrochloride; |
| 13. | N-(5-(3-hydroxypiperidin-1-yl)-2-methyl-2H-indazol-6-yl)-2-(2- |
| | methylpyridin-4-yl)oxazole-4-carboxamide hydrochloride; |
| 14. | N-(5-(3-fluoropiperidin-1-yl)-2-methyl-2H-indazol-6-yl)-2-(2- |
| | methylpyridin-4-yl)oxazole-4-carboxamide; |
| 15. | (S)-2-(2-acetamidopyridin-4-yl)-N-(5-(3-hydroxypyrrolidin-1- |
| | yl)-1-methyl-1H-indazol-6-yl)oxazole-4-carboxamide; |
| 16. | N-(5-(3-fluoropiperidin-1-yl)-1-methyl-1H-indazol-6-yl)-2-(2- |
| | methylpyridin-4-yl)oxazole-4-carboxamide; |
| 17. | N-(5-(4-hydroxypiperidin-1-yl)-1-methyl-1H-indazol-6-yl)-2-(2- |
| | methylpyridin-4-yl)oxazole-4-carboxamide; |
| 18. | (S)-2-(2-aminopyridin-4-yl)-N-(5-(3-hydroxypyrrolidin-1-yl)-1- |
| | methyl-1H-indazol-6-yl)oxazole-4-carboxamide; |
| 19. | N-(5-(4-fluoropiperidin-1-yl)-1-methyl-1H-indazol-6-yl)-2-(2- |
| | methylpyridin-4-yl)oxazole-4-carboxamide; |
| 20. | N-(5-(4-(hydroxymethyl)piperidin-1-yl)-1-methyl-1H-indazol- |
| | 6-yl)-2-(2-methylpyridin-4-yl)oxazole-4-carboxamide; |
| 21. | (S)-2-(2,6-dimethylpyridin-4-yl)-N-(5-(3-hydroxypyrrolidin-1- |
| | yl)-1-methyl-1H-indazol-6-yl)oxazole-4-carboxamide; |
| 22. | (R)-N-(5-(3-hydroxypyrrolidin-1-yl)-1-methyl-1H-indazol-6- |
| | yl)-2-(2-methylpyridin-4-yl)oxazole-4-carboxamide; |
| 23. | (S)-2-(2-aminopyridin-3-yl)-N-(5-(3-hydroxypyrrolidin-1-yl)- |
| | 1-methyl-1H-indazol-6-yl)oxazole-4-carboxamide |
| | Hydrochloride; |
| 24. | 6-((S)-3-hydroxypyrrolidin-1-yl)-N-(5-((R)-3-hydroxypyrrolidin- |
| | 1-yl)-1-methyl-1H-indazol-6-yl)picolinamide; |
| 25. | 6-((S)-3-hydroxypyrrolidin-1-yl)-N-(5-((S)-3-hydroxypyrrolidin- |
| | 1-yl)-1-methyl-1H-indazol-6-yl) picolinamide; |
| 26. | (S)-N-(5-(3-hydroxypyrrolidin-1-yl)-1-methyl-1H-indazol-6-yl)- |
| | 2-(1H-pyrrolo[2,3-b]pyridin-4-yl)oxazole-4-carboxamide; |
| 27. | (S)-N-(5-(3-hydroxypyrrolidin-1-yl)-1H-indazol-6-yl)-2-(2- |
| | methylpyridin-4-yl)oxazole-4-carboxamide; |
| 28. | (S)-2-(2-amino-3-fluoropyridin-4-yl)-N-(5-(3-hydroxypyrrolidin- |
| | 1-yl)-1-methyl-1H-indazol-6-yl)oxazole-4-carboxamide; |
| 29. | (R)-2-(2-aminopyridin-3-yl)-N-(5-(3-hydroxypyrrolidin-1-yl)-1- |
| | methyl-1H-indazol-6-yl)oxazole-4-carboxamide hydrochloride; |
| 30. | (S)-N-(5-(3-hydroxypyrrolidin-1-yl)-1-methyl-1H-indazol-6- |
| | yl)-2-(4-methylpiperazin-1-yl)oxazole-4-carboxamide; |
| 31. | (S)-N-(5-(3-hydroxypyrrolidin-1-yl)-1-methyl-1H-indazol-6- |
| | yl)-2-(piperazin-1-yl)oxazole-4-carboxamide hydrochloride; |
| 32. | (S)-N-(1-ethyl-5-(3-hydroxypyrrolidin-1-yl)-1H-indazol-6-yl)- |
| | 2-(2-methylpyridin-4-yl)oxazole-4-carboxamide hydrochloride; |
| 33. | (S)-N-(1-cyclopropyl-5-(3-hydroxypyrrolidin-1-yl)-1H- |
| | indazol-6-yl)-2-(2-methylpyridin-4-yl)oxazole-4-carboxamide |
| | hydrochloride; |
| 34. | (S)-N-(5-(3-hydroxypyrrolidin-1-yl)-1-methyl-1H-indazol-6- |
| | yl)-2-(1,2,3,6-tetrahydropyridin-4-yl)oxazole-4-carboxamide |
| | hydrochloride; |
| 35. | (S)-N-(5-(3-hydroxypyrrolidin-1-yl)-1-methyl-1H-indazol-6- |
| | yl)-2-(2-methylpyrimidin-4-yl)oxazole-4-carboxamide |
| | hydrochloride; |
| 36. | (S)-N-(5-(3-hydroxypyrrolidin-1-yl)-1-methyl-1H-indazol-6-yl)- |
| | 4-methyl-2-(2-methylpyridin-4-yl) oxazole-5-carboxamide |
| | hydrochloride; |
| 37. | (S)-N-(5-(3-hydroxypyrrolidin-1-yl)-1-methyl-1H-indazol-6- |
| | yl)-2-(piperidin-4-yl)oxazole-4-carboxamide hydrochloride; |
| 38. | N-(5-(3-hydroxy-8-azabicyclo[3.2.1]octan-8-yl)-1-methyl-1H- |
| | indazol-6-yl)-2-(2-methylpyridin-4-yl)oxazole-4-carboxamide; |
| 39. | (S)-N-(5-(3-hydroxypyrrolidin-1-yl)-1-methyl-1H-indazol-6- |
| | yl)-2-(2-methylpyridin-4-yl) oxazole-5-carboxamide; |
| 40. | N-(5-(4-hydroxy-4-(hydroxymethyl)piperidin-1-yl)-1-methyl-1H- |
| | indazol-6-yl)-2-(2-methylpyridin-4-yl)oxazole-4-carboxamide; |
| 41. | (S)-N-(5-(3-hydroxypyrrolidin-1-yl)-1-methyl-1H-indazol-6-yl)- |
| | 5-methyl-2-(2-methylpyridin-4-yl)oxazole-4-carboxamide; |
| 42. | (S)-2-(2-ethylpyridin-4-yl)-N-(5-(3-hydroxypyrrolidin-1-yl)-1- |
| | methyl-1H-indazol-6-yl)oxazole-4-carboxamide; |
| 43. | 2-(2-aminopyridin-4-yl)-N-(5-(4-(hydroxymethyl)piperidin-1- |
| | yl)-1,3-dimethyl-1H-indazol-6-yl)oxazole-4-carboxamide |
| | hydrochloride; |
| 44. | (S)-N-(5-(3-hydroxypyrrolidin-1-yl)-1-(piperidin-4- |
| | ylmethyl)-1H-indazol-6-yl)-2-(2-methylpyridin-4-yl)oxazole- |
| | 4-carboxamide hydrochloride; |
| 45. | N-(5-(4-(hydroxymethyl)piperidin-1-yl)-1,3-dimethyl-1H- |
| | indazol-6-yl)-2-(2-methylpyridin-4-yl)oxazole-4- |
| | carboxamide hydrochloride; |
| 46. | (S)-2-(2-cyclopropylpyridin-4-yl)-N-(5-(3-hydroxypyrrolidin-1- |
| | yl)-1-methyl-1H-indazol-6-yl)oxazole-4-carboxamide |
| | hydrochloride; |
| 47. | N-(5-(4-hydroxypiperidin-1-yl)-2-methyl-2H-indazol-6- |
| | yl)pyrazolo[1,5-a]pyrimidine-3-carboxamide hydrochloride; and |
| 48. | (S)-N-(5-(3-hydroxypyrrolidin-1-yl)-1-methyl-1H-indazol-6- |
| | yl)pyrazolo[1,5-a]pyrimidine-3-carboxamide hydrochloride; |

or a pharmaceutically acceptable salt or a stereoisomer thereof.

49. A pharmaceutical composition comprising at least one compound according to claim 1, or a pharmaceutically acceptable salt or a stereoisomer thereof, and a pharmaceutically acceptable carrier or excipient.

50. A method of treating an IRAK4 mediated disorder, disease, or condition in a subject, comprising administering a therapeutically effective amount of a compound according to claim 1.

51. The method of claim 50, wherein the IRAK4 mediated disorder, disease, or condition is selected from a cancer, a proliferative disorder, an inflammatory disorder, an autoimmune disease, a metabolic disorder, a hereditary disorder, a hormone-related disease, an immunodeficiency disorder, a condition associated with cell death, a destructive bone disorder, thrombin-induced platelet aggregation, liver disease and a cardiovascular disorder.

52. The method of claim 51, wherein the cancer or the proliferative disorder is selected from a solid tumor, benign or malignant tumor, carcinoma of the brain, kidney, liver, stomach, vagina, ovaries, gastric tumors, breast, bladder, colon, prostate, pancreas, lung, cervix, testis, skin, bone or thyroid; sarcoma, glioblastomas, neuroblastomas, multiple myeloma, gastrointestinal cancer, a tumor of the neck and head, an epidermal hyperproliferation, psoriasis, prostate hyperplasia, a neoplasia, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, papillary carcinoma, seminoma, melanoma; and hematological malignancies selected from leukemia, diffuse large B-cell lymphoma (DLBCL), activated B-cell-like DLBCL, chronic lymphocytic leukemia (CLL), chronic lymphocytic lymphoma, primary effusion lymphoma, Burkitt lymphoma/leukemia, acute lymphocytic leukemia, B-cell pro lymphocytic leukemia, lymphoplasmacytic lymphoma, Waldenstrom's macroglobulnemia (WM), splenic marginal zone lymphoma, intravascular large B-cell lymphoma, plasmacytoma and multiple myeloma.

53. The method of claim 51, wherein the inflammatory disorder is selected from ocular allergy, conjunctivitis, keratoconjunctivitis sicca, vernal conjunctivitis, allergic rhinitis, autoimmune hematological disorders, systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease, irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, primary biliary cirrhosis, uveitis, Sjogren's syndrome, interstitial lung fibrosis, psoriatic arthritis, systemic juvenile idiopathic arthritis, nephritis, vasculitis, diverticulitis, interstitial cystitis, glomerulonephritis, chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS) and osteoarthritis.

54. A method of treating a disease or condition associated with an MYD88 mutation, comprising administering to a subject in need thereof a therapeutically effective amount of a compound according to claim 1.

55. The method of claim 54, wherein the disease or condition associated with an MYD88 mutation is selected from a cancer, a proliferative disorder, an inflammatory disorder, an autoimmune disease, a metabolic disorder, a hereditary disorder, a hormone-related disease, an immunodeficiency disorder, a condition associated with cell death, a destructive bone disorder, thrombin-induced platelet aggregation, liver disease and a cardiovascular disorder.

56. The method of claim 54, wherein the disease or condition is ulcerative colitis.

57. The method of claim 54, wherein the disease or condition is a lymphoma.

58. The method of claim 54, wherein the disease or condition is a cancer selected from diffuse large B-cell lymphoma and Waldenstroem's macroglobulinemia.
